(19) United States
(12) Patent Application Publication
(10) Pub. No.: US 2023/0014055 A1
(43) Pub. Date: Jan. 19, 2023

(54) Treatment of Immune-Related Disorders, Kidney Disorders, Liver Disorders, Hemolytic Disorders, and Oxidative Stress-Associated Disorders Using NRH, NARH and Reduced Derivatives Thereof

(71) Applicant: MitoPower LLC, Polo Alto, CA (US)

(72) Inventors: G. Mani Subramanian, Los Altos Hills, CA (US); Gautham Tumkur Pranesh, Bangalore (IN); Gangadhara Ganapati, Palo Alto, CA (US); Nikhil Saji Zachariah, Bangalore (IN); K.S. Ajay Kumar, Bangalore (IN)

(21) Appl. No.: 17/842,158

(22) Filed: Jun. 16, 2022

Related U.S. Application Data
(60) Provisional application No. 63/236,974, filed on Aug. 25, 2021.

(30) Foreign Application Priority Data
Jun. 18, 2021 (IN) 202141027391

(51) Int. Cl. A61K 31/706; A61K 9/20

(52) U.S. Cl. CPC A61K 31/706; A61K 9/20

(57) ABSTRACT

The disclosure relates to in vivo and ex vivo uses of dihydronicotinamide riboside (NRH), dihydronicotinic acid riboside (NARH) and reduced derivatives thereof to treat immune-related disorders (e.g., systemic inflammatory response syndrome and sepsis), kidney disorders (e.g., acute kidney injury and hepatorenal syndrome [HRS]), liver disorders (e.g., acute liver failure and HRS), hemolytic disorders (e.g., hemolysis and hemolytic anemia), and disorders and conditions associated with oxidative stress, damage or injury (e.g., methemoglobinemia and anemia). NRH, NARH and reduced derivatives thereof can be used in vivo or ex vivo alone or in combination with one or more additional therapeutic agents, such as an anti-inflammatory agent or/and an antioxidant.

### CROSS REFERENCE TO RELATED APPLICATIONS

This application claims priority to Indian Provisional Application Serial No. 202141027391, filed Jun. 18, 2021 under 35 U.S.C. § 119 (b) and U.S. Provisional Application Ser. No. 63/236,974 filed Aug. 25, 2021 under 35 U.S.C. § 119 (e) both of which are incorporated by reference in their entirety.

### TECHNICAL FIELD

The disclosure relates to the use of dihydronicotinamide riboside (NRH), dihydronicotinic acid riboside (NARH) and reduced derivatives thereof to treat immune-related disorders, kidney disorders, liver disorders, hemolytic disorders, and disorders and conditions associated with oxidative stress, damage or injury.

### BACKGROUND

Systemic inflammatory response syndrome (SIRS) is an inflammatory state affecting the whole body as a consequence of an exaggerated immune response to a non-infectious or infectious insult. Sepsis is a closely related disorder in which the patient satisfies criteria for SIRS and has a suspected or proven infection. Complications of SIRS and sepsis include shock and dysfunction and failure of one or more organs. SIRS, sepsis or a complication thereof is one of the most common causes of death of critically ill patients in the intensive care unit (ICU), accounting for up to 50% of all such deaths, with the risk of death from SIRS or sepsis as high as 30%, that from severe SIRS or sepsis as high as 50% and that from shock/septic shock as high as 80%.

Increased systemic inflammation is a common cause of organ dysfunction, kidney failure and death in patients with decompensated cirrhosis. Hepatorenal syndrome (HRS) can be an acute complication of chronic liver disease (CL D) which is frequently accompanied by SIRS and characterized by liver dysfunction accompanied by portal hypertension and ascites (fluid accumulation in the abdomen) that culminate in a reactive vasoconstriction of the renal artery and acute kidney injury (AKI). About 10% of hospital patients with ascites, such as CLD-related ascites, have HRS. Type 1 HRS has a mortality rate greater than 50% over the short term, but treatments can stabilize the condition while the patients wait for a liver transplant. Type 2 HRS patients have a median survival of about 6 months unless they receive a liver transplant.

### SUMMARY

The disclosure relates to in vivo and ex vivo uses of dihydronicotinamide riboside (NRH), dihydronicotinic acid riboside (NARH) and reduced derivatives thereof to treat immune-related disorders, kidney disorders, liver disorders, hemolytic disorders, and disorders and conditions associated with oxidative stress, damage or injury. In some embodiments, the immune-related disorders are SIRS and sepsis, the kidney disorders are AKI and HRS, the liver disorders are alcoholic hepatitis, acute liver failure (ALF), acute-on-chronic liver failure (ACLF), cirrhosis and HRS, the hemolytic disorders are hemolysis and hemolytic anemnia, and the disorders and conditions associated with oxidative stress, damage or injury are methemoglobinemia and anemia. In some embodiments, reduced derivatives of NRH and NARH have Formula I, where R¹, R² and R³ are defined elsewhere herein:

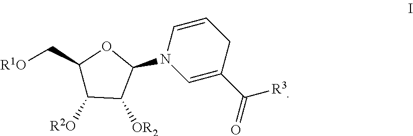

NRH, NARH and reduced derivatives thereof can be used in vivo or ex vivo alone or in combination with one or more additional therapeutic agents, such as an anti-inflammatory agent or/and an antioxidant.

### BRIEF DESCRIPTION OF THE DRAWINGS

FIG. 1 shows an exemplary process for synthesizing NRH, NARH and reduced derivatives thereof of Formula I which have the 5′-hydroxyl group, and optionally the 2′- and 3′-hydroxyl groups, of D-riboside derivatized.

FIG. 2 shows an exemplary process for synthesizing reduced derivatives of NRH and NARH of Formula I which have the 2′- and 3′-hydroxyl groups of D-riboside derivatized.

FIG. 3 shows an exemplary process for synthesizing reduced derivatives of NRH and NARH of Formula I which have the 2′-, 3′- and 5′-hydroxyl groups of D-riboside derivatized.

FIG. 4 shows that in an ex vivo polyclonal immune activation model, CD8⁺ T cells stimulated with anti-CD3 and anti-CD28 antibodies produced significantly or markedly more IFN-γ, TNF-α and IL-2 than unstimulated (US) CD8⁺ T cells, and NRH (MP-04) significantly reduced the production of IFN-γ, TNF-α and IL-2 in activated CD8⁺ T cells (p<0.05 in the Mann-Whitney U test).

FIG. 5 shows that peripheral blood mononuclear cells (PBMCs) from a healthy human donor stimulated with anti-CD3 and anti-CD28 antibodies had a markedly higher extracellular acidification rate (ECAR, a measure of glycolysis) than unstimulated PBMCs, and NRH (MP-04) significantly reduced ECAR in activated PBMCs.

FIGS. 6 and 7 show that incubation with NRH (MP-04) and NRH-triacetate (MP-40) for 24 hr significantly induced mitochondrial membrane depolarization in CD4⁺ and CD8⁺ T cells, respectively, unstimulated or stimulated with anti-CD3 and anti-CD28 antibodies.

FIGS. 8 and 9 show that incubation with NRH (MP-04) and NRHTA (MP-40) for 24 hr reduced cell death including apoptosis of CD4⁺ and CD8⁺ T cells, respectively, with depolarized mitochondria and unstimulated or stimulated with anti-CD3 and anti-CD28 antibodies.

FIG. 10 shows that both NRH (MP-04) and NRH-triacetate (MP-40), but neither NR (MP-02) nor NR-triacetate (MP-39) at any concentration tested, reduced H₂O₂-induced hemolysis in an in vitro assay.

FIG. 11 shows that 10 mM H₂O₂ caused oxidative changes to hemoglobin which reduced the amplitude of absorbance peaks at 576 nm, 540 nm, 434 nm, 348 nm and 270 nm.

FIG. 12A-C shows that pre-incubation of RBCs with 1, 10 and 100 μM, respectively, of NRH (MP04), but not with NR (MP02), protected hemoglobin from 1 mM H₂O₂-induced oxidative changes, as pre-incubation with NRH increased the amplitude of absorbance peaks at 576 nm, 540 nm, 434 nm, 348 nm and 270 nm.

FIG. 13 shows that exposure of RBCs to 1 mM H₂O₂ significantly reduced the A₅₇₆/A₆₃₀ ratio (a measure of the hemoglobin/methemoglobin ratio), and treatment of RBCs exposed to 1 mM H₂O₂ with 1 μM or 100 μM NRH (MP-04) restored the A₅₇₆/A₆₃₀ ratio.

FIGS. 14A and B shows that 30 min and 6 hr, respectively, of incubation with NRH (MP04) at 100 and 1000 μM significantly increased the NADH/NAD⁺ ratio in HEK293 cells exposed to H₂O₂, while NR (MP02) at all tested concentrations did not significantly affect the ratio.

FIG. 15 shows that both NRH (MP04) and NRH-triacetate (MP40) were much more stable in human serum than NR (MP02) in an in vitro assay (*=p<0.05 for NR versus NRH and NRH-triacetate; #=p<0.05 for NRH-triacetate versus NRH).

FIG. 16A-C shows that a single intraperitoneal injection of NRH (MP-04) into a Wistar Han rat at a dose of 500 mg/kg resulted in increased concentrations of NRH in whole blood, the kidney and the liver, respectively, after 4 hr as compared to the corresponding concentrations in a Wistar Han rat intraperitoneally injected with vehicle. The “area ratio NRH/IS” is the ratio of the peak area of NRH to the peak area of internal standard (tolbutamide).

### GENERAL STATEMENTS

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein can be employed in practicing the disclosure. It is also understood that every embodiment of the disclosure can optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is further understood that the disclosure of a numerical range is a specific disclosure of all the possible subranges and all the possible individual numbers (whether whole numbers or fractions) within that range regardless of the breadth of that range.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment or compound found within the prior art, can be explicitly excluded from the claims, regardless of whether or not the specific exclusion is recited in the specification.

It is further understood that the present disclosure encompasses salts, solvates, hydrates, clathrates and polymorphs of all of the compounds disclosed herein. The specific recitation of “salts”, “solvates”, “hydrates”, “clathrates” or “polymorphs” with respect to a compound or a group of compounds in certain instances of the disclosure shall not be interpreted as an intended omission of any of these forms in other instances of the disclosure where the compound or the group of compounds is mentioned without recitation of any of these forms, unless stated otherwise or the context clearly indicates otherwise.

All patent literature and all non-patent literature cited herein are incorporated herein by reference in their entirety to the same extent as if each patent literature or non-patent literature were specifically and individually indicated to be incorporated herein by reference in its entirety.

### Definitions

Unless defined otherwise or clearly indicated otherwise by their use herein, all technical and scientific terms used herein have the same meaning as commonly understood by those of ordinary skill in the art to which this application belongs.

As used in the specification and the claims, the indefinite articles “a” and “an” and the definite article “the” can include plural referents as well as singular referents unless specifically stated otherwise or the context clearly indicates otherwise.

The term “exemplary” as used herein means “serving as an example, instance or illustration”. Any embodiment or feature characterized herein as “exemplary” should not be construed as preferred or advantageous over other embodiments or features.

In some embodiments, the term “about” or “approximately” means within ±10% or 5% of the given value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

A “modulator” of, e.g., a receptor or enzyme can be an activator or inhibitor of that receptor or enzyme, and can increase or reduce the activity or/and the level of that receptor or enzyme.

The term “parenteral” refers to a route of administration other than through the alimentary canal, such as by injection, infusion or inhalation. Parenteral administration includes without limitation subcuticular, intradermal, subcutaneous, intravascular, intravenous, intra-arterial, intramuscular, intracardiac, intraperitoneal, intracavitary, intra-articular, intracapsular, subcapsular, intra-orbital, transtracheal, intrasternal, intrathecal, intramedullary, intraspinal, subarachnoid and topical administrations. Topical administration includes without limitation dermal/epicutaneous, transdermal, mucosal, transmucosal, intranasal (e.g., by nasal spray or drop), ocular (e.g., by eye drop), pulmonary (e.g., by oral or nasal inhalation), buccal, sublingual, rectal (e.g., by suppository), and vaginal (e.g., by suppository).

The term “pharmaceutically acceptable” refers to a substance (e.g., an active ingredient or an excipient) that is suitable for use in contact with the cells, tissues and organs of a subject without excessive irritation, allergic response, immunogenicity and toxicity, is commensurate with a reasonable benefit/risk ratio, and is effective for its intended use. A “pharmaceutically acceptable” excipient or carrier of a pharmaceutical composition is also compatible with the other ingredients of the composition

The term “therapeutically effective amount” refers to an amount of a compound that, when administered to a subject or used ex vivo, is sufficient to prevent, reduce the risk of developing, delay the onset of, slow the progression of or cause regression of the medical condition being treated, or to alleviate to some extent the medical condition or one or more symptoms or complications of that condition, at least in some fraction of the subjects taking that compound or undergoing ex vivo treatment with that compound. The term “therapeutically effective amount” also refers to an amount of a compound that is sufficient to elicit the biological or medical response of a cell, tissue, organ, system, animal or human which is sought by a researcher, veterinarian, medical doctor or clinician.

The terms “treat”, “treating” and “treatment” include alleviating, ameliorating, reducing the severity or frequency of, slowing or inhibiting the progress of, reversing or abrogating a medical condition or one or more symptoms or complications associated with the condition, and alleviating, ameliorating or eradicating one or more causes of the condition. Reference to “treatment” of a medical condition includes prevention of the condition. The terms “prevent”, “preventing” and “prevention” include precluding, reducing the risk of developing and delaying the onset of a medical condition or one or more symptoms or complications associated with the condition.

The term “medical conditions” (or “conditions” for short) includes diseases and disorders. The terms “diseases” and “disorders” are used interchangeably herein.

The term “subject” refers to an animal, including but not limited to a mammal, such as a primate (e.g., a human, a chimpanzee or a monkey), a rodent (e.g., a rat, a mouse, a guinea pig, a gerbil or a hamster), a lagomorph (e.g., a rabbit), a bovine (e.g., a cattle), a suid (e.g., a pig), a caprine (e.g., a sheep), an equine (e.g., a horse), a canine (e.g., a dog) or a feline (e.g., a cat). The terms “subject” and “patient” are used interchangeably herein in reference, e.g., to a mammalian subject, such as a human subject.

The disclosure encompasses salts, solvates, hydrates, clathrates and polymorphs of the compounds described herein. A “solvate” of a compound comprises a stoichiometric or non-stoichiometric amount of a solvent molecule (e.g., water, acetone or an alcohol [e.g., ethanol]) bound non-covalently to the compound. A “hydrate” of a compound comprises a stoichiometric or non-stoichiometric amount of water molecule bound non-covalently to the compound. A “clathrate” of a compound contains molecules of a substance (e.g., a solvent) enclosed in a crystal structure of the compound. A “polymorph” of a compound is a crystalline form of the compound.

The term “alkyl” refers to a linear (straight chain) or branched, saturated monovalent hydrocarbon radical, which can optionally be substituted with one or more substituents. The term “lower alkyl” refers to a linear C₁-C₆ or branched C₃-C₆ alkyl group. Lower alkyl groups include without limitation methyl, ethyl, propyl (including n-propyl and isopropyl), butyl (including all isomeric forms, such as n-butyl, isobutyl, sec-butyl and tert-butyl), pentyl (including all isomeric forms, such as n-pentyl and isopentyl), and hexyl (including all isomeric forms, such as n-hexyl).

The term “alkenyl” refers to an alkyl group having one or more C═C double bonds. An alkenyl group can optionally be substituted with one or more substituents.

The term “acyl” refers to a —C(═O)-alkyl or —C(═O)-alkenyl group.

The term “cycloalkyl” refers to a cyclic saturated, bridged or non-bridged monovalent hydrocarbon radical, which can optionally be substituted with one or more substituents. C₃-C₆ cycloalkyl groups include cyclopropyl, cyclobutyl, cyclopentyl and cyclohexyl.

The term “heterocyclyl” or “heterocyclic” refers to a monocyclic non-aromatic group or a multicyclic group that contains at least one non-aromatic ring, wherein at least one non-aromatic ring contains one or more heteroatoms independently selected from O, N and S. The non-aromatic ring containing one or more heteroatoms may be attached or fused to one or more saturated, partially unsaturated or aromatic rings. A heterocyclyl or heterocyclic group can optionally be substituted with one or more substituents. 3- to 6-membered, nitrogen-containing heterocyclic rings include without limitation aziridinyl, azetidinyl, pyrrolidinyl, piperidinyl, piperazinyl and morpholinyl.

### DETAILED DESCRIPTION OF THE DISCLOSURE

Therapeutic Uses of NRH, NARH and Reduced Derivatives Thereof

The disclosure provides for in vivo and ex vivo uses of dihydronicotinamide riboside (NRH), dihydronicotinic acid riboside (NARH) and reduced derivatives thereof (e.g., those of Formula I [infra]) to treat immune-related disorders, kidney disorders, liver disorders, hemolytic disorders, and disorders and conditions associated with oxidative stress, damage or injury. NARH can be in the carboxylic acid form or the carboxylate form. Some embodiments relate to a method of treating an immune-related disorder, a kidney disorder, a liver disorder, a hemolytic disorder, or a disorder or condition associated with oxidative stress, damage or injury, comprising administering to a subject in need of treatment a therapeutically effective amount of, or contacting cells or biological fluid from a subject in need of treatment ex vivo with, NRH, NARH or a reduced derivative thereof, or a pharmaceutically acceptable salt, solvate, hydrate, clathrate, polymorph or stereoisomer thereof. The cells or biological fluid from the subject contacted ex vivo with NRH, NARH or a reduced derivative thereof are characterized by or at risk of oxidative stress, damage or injury, or/and the subject suffers from an immune-related disorder, a kidney disorder, a liver disorder, a hemolytic disorder, or a disorder or condition associated with oxidative stress, damage or injury.

In some embodiments, NRH, NARH or a reduced derivative thereof (e.g., that of Formula I) is used in vivo or ex vivo to treat an immune-related disorder. Immune-related disorders include without limitation disorders associated with overactivation of the immune system or immune function, inflammatory disorders, autoimmune disorders, and allergic disorders. Certain disorders may fall within multiple categories of such disorders. For example, systemic inflammatory response syndrome (SIRS) and sepsis, and many autoimmune disorders and allergic disorders, may be regarded as disorders associated with overactivation of the immune system or immune function and inflammatory disorders.

NRH, NARH and reduced derivatives thereof can suppress aberrant immune cell activation and aberrant inflammatory immune responses (e.g., as a result of a cytokine storm) by suppressing glycolysis. Suppression of glycolysis results in quiescence of cells whose energy metabolism is predominantly glycolytic, including activated or overactivated immune cells (e.g., B cells, T cells, natural killer cells and macrophages), activated fibroblasts (involved in fibrosis), and tumor and cancer cells. Because the anabolic pentose phosphate pathway (PPP) generates ribose 5-phosphate, a precursor for synthesis of nucleotides, and the PPP begins with dehydrogenation of glucose-6-phosphate, the first intermediate produced by glycolysis, suppression of glycolysis also suppresses the PPP and hence activation, growth and proliferation of immune cells, fibroblasts and tumor/cancer cells. The immune system can become overactive in response to, e.g., a host agent (such as in an autoimmune disorder) or a foreign agent (e.g., a pathogen). In certain embodiments, a disorder associated with overactivation of the immune system or immune function is caused by a pathogenic (e.g., bacterial or viral) infection, such as one with a coronavirus (e.g., SARS-CoV-2 responsible for COVID-19).

Disorders associated with overactivation of the immune system or immune function include without limitation disorders caused by or resulting from a cytokine storm. SIRS (which may have a non-infectious or infectious cause) is typically, and sepsis (which results from an infection) is often, associated with a cytokine storm. In a cytokine storm, an overactive response of the adaptive or/and innate immune system(s) to an insult brings about an excessive and uncontrolled release of pro-inflammatory cytokines, which can result in severe inflammation, severe damage and injury to tissues and organs, and death of the subject. Cytokine storms can be incited by non-infectious insults (e.g., graft-versus-host disease and medications such as theralizumab) and infectious insults, including infections with bacteria (e.g., group A Streptococcus) and viruses (e.g., cytomegalovirus and Epstein-Barr virus), especially respiratory viruses (e.g., influenza B, H1N1 influenza, H5N1 influenza, parainfluenza, SARS-CoV-1 and SARS-CoV-2). The respiratory viruses can invade lung epithelial cells and alveolar macrophages to produce viral nucleic acid, which stimulates the infected cells to release cytokines and chemokines, activating macrophages, dendritic cells and other immune cells. About 70% of COVID-19 deaths are due to acute respiratory distress syndrome (ARDS) caused by a cytokine storm resulting from a SARS-CoV-2 infection.

In some embodiments, the immune-related disorder is SIRS or sepsis. SIRS is a serious condition characterized by systemic inflammation resulting from the body's response to a non-infectious or infectious insult. The systemic inflammation typically results from a cytokine storm in which an exaggerated response of the adaptive or/and innate immune system(s) to the insult brings about an excessive and uncontrolled release of pro-inflammatory cytokines. Non-infectious causes of SIRS include without limitation trauma, burns, surgery, ischemia, pulmonary embolism, cardiac tamponade, heart failure, neurogenic shock, low blood volume, adrenal insufficiency, thyrotoxicosis (including hyperthyroidism), hemorrhage, aortic aneurysm, anaphylaxis, acute inflammation, pancreatitis, pneumonitis (e.g., chemical pneumonitis), alcoholic hepatitis, malignancies, medications (e.g., theralizumab), drug overdose, and substance (e.g., alcohol) abuse. Infectious causes of SIRS include, but are not limited to, infections by bacteria, parasites (e.g., Plasmodium such as P. falciparum responsible for most cases of severe malaria, and amebas/ameboids such as Acanthameba responsible for granulomatous amebic encephalitis and brain abscesses and Entamoeba [e.g., E. histolytica] responsible for amebiasis [amebic dysentery] and amebic abscesses [e.g., in the liver]), and viruses (e.g., SARS-CoV-2 responsible for Covid-19).

Many patients with alcoholic hepatitis manifest symptoms of SIRS (e.g., leukocytosis and fever) without any identifiable infection, and the SIRS may be secondary to sterile inflammation, namely, an inflammatory response in the absence of a pathogen. The liver of patients with alcoholic hepatitis is characterized by a marked overexpression of pro-inflammatory cytokines such as IL-8, which correlates with short-term mortality and suggests that inflammatory mediators produced by the injured liver are involved in the development of SIRS in patients with alcoholic hepatitis.

SIRS is closely related to sepsis, in which patients satisfy criteria for SIRS and have a suspected or proven infection. Sepsis can be caused by many microbes, including bacteria (e.g., gram-positive bacteria such as staphylococci and Streptococcus pyogenes, and gram-negative bacteria such as Klebsiella, Escherichia coli and Pseudomonas aeruginosa), fungi (e.g., pathogenic yeasts such as Candida, and molds such as Aspergillus, Fusarium and Mucor), parasites (e.g., Plasmodium, Schistostoma and Echinococcus), and viruses (e.g., SARS-CoV-2). Upon detection of microbial antigens, the systemic immune system is activated. Immune cells recognise pathogen-associated molecular patterns as well as damage-associated molecular patterns from damaged tissues, triggering an uncontrolled immune response involving recruitment of leukocytes all over the body, not only to the specific site of infection, excessive and uncontrolled release of pro-inflammatory cytokines, and damage to healthy tissues caused by the overactive immune response which can persist after removal of the infectious agent. The early phase of sepsis characterized by excessive inflammation may be followed by a phase of reduced functioning of the immune system due in part to apoptosis of a variety of immune cells, and ultimately multiple organ failure.

In addition to systemic, excessive inflammation, SIRS and sepsis are characterized by increased oxidative stress and increased metabolic stress. See, e.g., V. Mishra, Clin. Lab., 53:199-209 (2007); and J. Macdonald et al., Brit. J. Anaesthesia, 90:221-232 (2003).

SIRS and sepsis often induce serious complications such as dysfunction or failure of one or more organs or organ systems, in which case the SIRS or sepsis is deemed “severe”, or/and shock or septic shock. Complications of SIRS and sepsis include without limitation respiratory dysfunction and failure (e.g., acute respiratory distress syndrome [ARDS]), liver dysfunction and failure (e.g., acute liver failure [ALF], acute-on-chronic liver failure [ACLF], chronic liver failure [CLF], chronic liver disease [CLD], cirrhosis and hepatorenal syndrome [HRS]), kidney dysfunction and failure (e.g., acute kidney injury [AKI], chronic kidney disease [CKD], end-stage kidney disease [ESKD] and HRS), cardiovascular dysfunction and failure (e.g., systolic or/and diastolic heart failure, hypotension, shock/septic shock, intravascular hemolysis, and disseminated intravascular coagulation), encephalopathy, multiple organ dysfunction syndrome (MODS) and multiple organ failure (MOF).

Systemic inflammation plays an important role in the development of complications of portal hypertension in cirrhosis. SIRS and sepsis frequently lead to redistribution of renal blood flow, resulting in ischemia and subsequent tubular injury. HRS-AKI can occur in an acute setting (e.g., ALF or ACLF) due to excessive release of pro-inflammatory cytokines or/and chemokines, which can cause renal damage (e.g., renal tubular damage such as acute tubular necrosis) and circulatory dysfunction (e.g., worsening of systemic vasodilation).

In a highly relevant ex vivo polyclonal immune activation model of SIRS, surprisingly both NRH and its reduced derivative NRH-triacetate (NRHTA), but not the oxidized form nicotinamide riboside (NR), exerted therapeutic effects (Examples 2-4). Unlike NR (data not shown), NRH reduced glycolysis (a metabolic hallmark of immune-cell activation) in peripheral blood mononuclear cells (PBMCs, which include monocytes and lymphocytes including T cells, B cells and natural killer cells) stimulated with anti-CD3 and anti-CD28 antibodies, NRH and NRHTA (data not shown) reduced production of pro-inflammatory cytokines (e.g., tumor necrosis factor-alpha [TNF-α], interleukin-2 [IL-2] and interferon-gamma [IFN-γ]) by CD8⁺ T cells (and CD4⁺ T cells [data not shown]) stimulated with anti-CD3 and anti-CD28 antibodies, NRH and NRHTA induced mitochondrial membrane depolarization in unstimulated and stimulated CD4⁺ and CD8⁺ T cells, and NRH and NRHTA reduced cell death including apoptosis of unstimulated and stimulated CD4⁺ and CD8⁺ T cells with depolarized mitochondria. Suppression of glycolysis in immune cells also suppresses the anabolic pentose phosphate pathway and consequently immune-cell activation and proliferation and an overactive immune response. Excessive reactive oxygen species (ROS) generated in the mitochondria can induce apoptosis through the caspase-mediated intrinsic (mitochondrial) pathway, and mitochondrial membrane depolarization can reduce mitochondrial production of ROS. Besides inducing mitochondrial membrane depolarization, NRH and NRHTA can decrease oxidative stress by increasing the NADH (reducing agent)/NAD⁺ (oxidizing agent) ratio and thereby improve cellular redox (reduction-oxidation) balance. In addition, decreasing oxidative stress decreases inflammation because oxidants (e.g., ROS) and oxidized molecules (e.g., oxidized lipids) can be highly inflammatory. Moreover, decreasing oxidative stress decreases oxidative damage to red blood cells and prevents hemolysis, which can occur in or lead to SIRS or sepsis. Hemolysis can lead to systemic inflammation and vasomotor dysfunction, and hence compromised hemodynamics and shock. Therefore, NRH, NARH and reduced derivatives thereof can exert therapeutic effects against SIRS, sepsis and complications thereof through multiple mechanisms of action, including inhibition of immune-cell activation, production of pro-inflammatory cytokines, oxidative stress, cell death including apoptosis, and hemolysis.

In some embodiments, NRH, NARH or a reduced derivative thereof is used in vivo or ex vivo to treat SIRS or sepsis or a complication thereof caused by or resulting from an infection with a bacterium (e.g., a gram-negative or gram-positive bacterium, a Mycobacterium or a gut bacterium), a fungus or a virus. In certain embodiments, the infection is a viral infection, such as a SARS-CoV-2 infection. SARS-CoV-2 infection in children can cause a closely related disorder called multisystem inflammatory syndrome in children (MIS-C) or pediatric inflammatory multisystem syndrome (PIMS).

In addition to treating a subject with existing SIRS or sepsis or a complication thereof, NRH, NARH or a reduced derivative thereof can be used in vivo or ex vivo to prevent, reduce the risk of developing or slow progression to SIRS or sepsis or a complication thereof. For example, NRH, NARH or a reduced derivative thereof can be used in vivo or ex vivo to prevent the generation of a cytokine storm, immune-mediated inflammatory damage to lung cells (e.g., alveolar cells) and progression of a respiratory viral infectious disorder such as COVID-19 to SIRS or sepsis or a complication thereof (e.g., ARDS). As another example, NRH, NARH or a reduced derivative thereof can be used in vivo or ex vivo to prevent progression of an acute inflammatory disorder (e.g., pneumonia, peritonitis, meningitis or cellulitis), whether or not caused by an infection such as a bacterial or viral infection, to SIRS or sepsis or a complication thereof.

Inflammatory disorders include without limitation SIRS, sepsis, neuroinflammation (e.g., neuritis [e.g., ocular neuritis and peripheral neuritis], encephalomyelitis [e.g., autoimmune encephalomyelitis], Alzheimer's disease and multiple sclerosis), meningitis, muscle disorders (e.g., myositis), gastrointestinal disorders {e.g., gastritis, colitis (e.g., mucous colitis, ulcerative colitis [UC] and necrotizing enterocolitis), inflammatory bowel disease (IBD, including UC and Crohn's disease), irritable bowel syndrome, and celiac disease}, peritonitis, pancreatitis (acute and chronic), kidney disorders (e.g., nephritis, glomerulonephritis, AKI and CKD), liver disorders (e.g., hepatitis, non-alcoholic and alcoholic steatohepatitis, cirrhosis and CLD), MODS (e.g., secondary to septicemia or trauma), metabolic disorders (e.g., diabetes [e.g., types 1 and 2 diabetes and juvenile-onset diabetes] and metabolic syndrome), cardiac disorders (e.g., myocarditis, non-ischemic cardiomyopathy, myocardial infarction and congestive heart failure), vascular disorders (e.g., vasculitis, atherosclerosis, stroke, peripheral artery disease and shock), reperfusion injury (e.g., due to myocardial ischemia, cerebral ischemia, cardiopulmonary bypass, renal ischemia or kidney dialysis), airway disorders (e.g., rhinitis [e.g., allergic rhinitis], esophagitis, asthma, acute and chronic lung injury, ARDS, bronchitis [e.g., chronic bronchitis], pneumonitis, pneumonia and chronic obstructive pulmonary disease [COPD]), rheumatic disorders {e.g., arthritis (e.g., osteoarthritis [degenerative joint disease], rheumatoid arthritis, juvenile arthritis, psoriatic arthritis, gout, axial spondyloarthritis and ankylosing spondylitis) and diffuse connective tissue disorders (e.g., systemic lupus erythematosus [SLE], Sjögren syndrome, and localized and systemic scleroderma)}, skin disorders (e.g., dermatitis/eczema, pemphigoid, psoriasis, urticaria, dermatosis with acute inflammatory components, cellulitis and sunburn), eye disorders (e.g., conjunctivitis, optic neuritis, retinitis, uveitis and age-related macular degeneration [AMD]), hypertension, endometriosis, dysmenorrhea (menstrual cramps), graft-versus-host disease, and transplant rejection.

Autoimmune disorders include without limitation nervous system disorders (e.g., multiple sclerosis and Guillain-Barre syndrome [GBS]), neuromuscular disorders (e.g., GBS and myasthenia gravis), gastrointestinal disorders (e.g., ulcerative colitis and celiac disease), liver disorders (e.g., autoimmune hepatitis), metabolic disorders (e.g., type 1 diabetes, Grave's disease [which causes hyperthyroidism], and Hashimoto's thyroiditis [which causes hypothyroidism]), rheumatic disorders (e.g., arthritis [e.g, rheumatoid arthritis and juvenile arthritis] and diffuse connective tissue disorders [e.g., SLE, Sjögren syndrome, and localized and systemic scleroderma]), skin disorders (e.g., pemphigus, pemphigoid and psoriasis), and anemias (e.g., aplastic anemia and autoimmune hemolytic anemia).

Allergic disorders include without limitation anaphylaxis, allergic asthma, allergic rhinitis, allergic atopic dermatitis/eczema, allergic contact dermatitis (e.g., urushiol-induced contact dermatitis after contact with poison ivy, eastern poison oak, western poison oak or poison sumac), and allergy caused by foods (e.g., cow's milk, soy, eggs, wheat, peanuts, tree nuts, fish and shellfish/crustaceans), medications (e.g., penicillins), latex, insect bites (e.g., by mosquitoes and ticks) and insect stings (e.g., by ants, bees, hornets and wasps).

In further embodiments, NRH, NARH or a reduced derivative thereof (e.g., that of Formula I) is used in vivo or ex vivo to treat a fibrotic disorder. Suppression of glycolysis in fibroblasts suppresses activation and proliferation of fibroblasts. Moreover, inhibition of inflammation inhibits fibrosis, because inflammation is a major stimulant of fibrosis. Fibrotic disorders include without limitation cardiomyopathy (e.g., ischemic and non-ischemic cardiomyopathy, diabetic cardiomyopathy and uremic cardiomyopathy), cardiac fibrosis, myocardial fibrosis, collagen-vascular diseases (e.g., arterial stiffness and vascular fibrosis), atherosclerosis, chronic heart failure, diabetic nephropathy, renal fibrosis (e.g., renal tubulointerstitial fibrosis), CKD, liver fibrosis, cirrhosis, NASH, ASH, CLD, liver failure (e.g., CLF), pulmonary fibrosis (e.g., idiopathic pulmonary fibrosis [IPF], connective tissue disease-related pulmonary fibrosis and radiation-induced pulmonary fibrosis), cystic fibrosis, scleroderma (e.g., localized scleroderma and systemic scleroderma/systemic sclerosis), and endometriosis.

In other embodiments, NRH, NARH or a reduced derivative thereof (e.g., that of Formula I) is used in vivo or ex vivo to treat a kidney disorder. Kidney disorders include without limitation nephritis, glomerulonephritis, nephritic syndrome, nephrosis, glomerulonephrosis, nephrotic syndrome, renal fibrosis (e.g., renal tubulointerstitial fibrosis), AKI (with pre-renal, instrinsic renal or post-renal causes), CKD, ESKD and HRS (types 1 and 2). AKI is sometimes referred to as acute renal failure (ARF). CKD includes chronic renal failure (CRF). In some embodiments, the kidney disorder is AKI or HRS.

Acute kidney injury (AKI) is a rapid decline in kidney function that develops within 7 days, as shown by an increase in serum creatinine level or/and a reduction in urine output (oliguria). Causes of AKI can be pre-renal (due to reduced blood flow to the kidneys that reduces the glomerular filtration rate [GFR]), intrinsic renal (due to damage to the kidneys themselves), or post-renal (due to blockage of urine flow). Pre-renal causes of AKI include, e.g., sepsis, low blood pressure, low blood volume (e.g., dehydration), excessive blood loss, cardiogenic shock, heart failure (leading to cardiorenal syndrome), HRS in the context of cirrhosis, local changes to the blood vessels supplying the kidney (e.g., renal artery stenosis and renal vein thrombosis), and certain medications such as angiotensin-converting enzyme (ACE) inhibitors, antibiotics (e.g., aminoglycosides and penicillins), NSAIDs (e.g., diclofenac, ibuprofen, indometacin and naproxen) and paracetamol (acetaminophen). Intrinsic renal causes of AKI include, e.g., lupus nephritis, glomerulonephritis, acute interstitial nephritis, acute tubular necrosis, crush injury, rhabdomyolysis, tumor lysis syndrome, contrast dyes (e.g., iodinated contrasts) used for imaging, and certain medications such as antibiotics (e.g, gentamicin), chemotherapeutics and calcineurin inhibitors (e.g., tacrolimus). Post-renal causes of AKI include, e.g., kidney stones, bladder stones, neurogenic bladder, benign prostatic hyperplasia (prostate enlargement), narrowing of the urethra, obstructed urinary catheter, cancer of the bladder, prostate or ureters, and certain medications such as anticholinergics. AKI increases the risk of developing CKD 9-fold and can lead to complications such as metabolic acidosis, uremia, hyperkalemia (high potassium level in the blood can cause abnormal heart rhythms), changes in bodily fluid balance, pulmonary edema, effects on other organ systems, and death. About 5-10% of AKI patients never regain full kidney function and develop ESKD, and thus require lifelong hemodialysis or a kidney transplant. AKI and CKD are associated with oxidative stress, inflammation and apoptosis. See, e.g., A. Tomsa et al., PeerJ, 7:e8046, DOI 10.7717/peerj.8046 (2019).

Hepatorenal syndrome (HRS) involves rapid deterioration in liver and kidney function. HRS usually occurs when liver function deteriorates rapidly due to an insult such as a bacterial infection, bleeding in the upper gastrointestinal tract, acute alcoholic hepatitis or overuse of a diuretic medication. Deteriorating liver function or liver disease results in release of vasoactive factors that cause dilation of blood vessels in the splanchnic circulation (which supplies the intestines) and constriction of blood vessels of the kidneys, which reduces blood flow to the kidneys and hence the GFR and causes dysfunction/failure of the kidneys in the absence of a significant abnormality in kidney morphology or histology. HRS occurs most commonly in subjects with cirrhosis (especially alcoholic cirrhosis with concomitant alcoholic hepatitis), and less commonly in the absence of cirrhosis in subjects with alcoholic hepatitis or fulminant liver failure. Spontaneous bacterial peritonitis (infection of ascites fluid) is the most common precipitant of HRS in subjects with cirrhosis. Acute IRS is sometimes referred to as AKI-HRS, and chronic HRS as CKD-HRS. SIRS is frequently a prominent feature of AKI-HRS. The two forms of HRS are type 1 and type 2. Both types of IRS involve deterioration in kidney function, as shown by elevated creatinine level in the serum or/and by reduced clearance of creatinine in the urine. Type 1 HRS is characterized by rapidly progressive decline in kidney function, and is typically associated with an inciting event. In contrast, type 2 HRS is slower in onset and progression of kidney dysfunction, and is typically not associated with an inciting event. Most type 2 HRS patients have portal hypertension and diuretic-resistant ascites (fluid accumulation in the abdomen), where the kidneys are unable to excrete sufficient sodium to clear the fluid even with the use of diuretic medications, before they develop deterioration in kidney function. HRS is usually fatal without a liver transplant, although treatments such as medications (e.g., a vasopressor or/and an inotrope) and interventions (e.g., hemodialysis or/and liver dialysis) can prevent worsening of type 1, but not type 2, HRS while the patients wait for a liver transplant. Type 2 HRS patients have a median survival of about 6 months unless they receive a liver transplant. HRS is associated with oxidative stress, inflammation and apoptosis. See, e.g., V. Nickovic et al., Renal Failure, 40:340-349 (2018).

In additional embodiments, NRH, NARH or a reduced derivative thereof (e.g., that of Formula I) is used in vivo or ex vivo to treat a liver disorder. Liver disorders include without limitation hepatitis (alcoholic and non-alcoholic), alcoholic liver disease (ALD), non-alcoholic fatty liver disease (NAFLD), alcoholic steatohepatitis (ASH), non-alcoholic steatohepatitis (NASH), liver fibrosis, cirrhosis (alcoholic and non-alcoholic, such as due to NAFLD or chronic hepatitis B or C), hepatotoxicity (e.g., drug-induced liver injury [DILI]), acute and chronic liver injury, ALF, ACLF, CLF, acute liver disease, CLD, and HRS (types 1 and 2). In some embodiments, the liver disorder is alcoholic hepatitis, cirrhosis, DILI, acute liver injury, ALF, ACLF or HRS. CLD can be caused by, e.g., a virus (e.g., hepatitis B, hepatitis C, cytomegalovirus or Epstein-Barr virus), a parasite (e.g., schistosomiasis), a hepatotoxic agent (e.g., alcohol) or drug (e.g., methotrexate), or a metabolic disorder (e.g., NAFLD, NASH, hemochromatosis or Wilson's disease). ALD encompasses liver manifestations of alcohol overconsumption, including fatty liver, alcoholic hepatitis, and chronic hepatitis with liver fibrosis or cirrhosis. ALD is associated with oxidative stress, inflammation and apoptosis. See, e.g., H. Tan et al., World J. Hepatol., 12:332-349 (2020).

NAFLD, the most common liver disorder in developed countries, is characterized by fatty liver that occurs when fat, in particular free fatty acids and triglycerides, accumulates in liver cells (hepatic steatosis) due to causes other than excessive alcohol consumption, such as nutrient overload, high caloric intake and metabolic dysfunction (e.g., hyperlipidemia and impaired glucose control). A liver can remain fatty without disturbing liver function, but a fatty liver can progress to become NASH, a condition in which steatosis is accompanied by inflammation, hepatocyte ballooning and cell injury with or without fibrosis of the liver. Fibrosis is the strongest predictor of mortality from NASH. NASH is the most extreme form of NAFLD. NASH is a progressive disease, with about 20% of patients developing cirrhosis of the liver and about 10% dying from a liver disease, such as cirrhosis or a liver cancer (e.g., hepatocellular carcinoma). NAFLD and hepatic and extrahepatic dysfunctions thereof are associated with oxidative stress, inflammation and apoptosis. See, e.g., A. Gonzalez et al., Oxid. Med. Cell. Longevity, 2020:1617805 (2020).

Hepatotoxicity in general is chemical-induced liver damage and includes drug-induced liver injury (DILI). DILI can cause acute and chronic liver disease, and is responsible for about 50% of ALF cases. ALF is characterized by catastrophic mitochondrial failure and generation of reactive oxygen species (ROS) leading to massive cell death (often about 70-90% of liver cells die). Chemicals (including medications) that can cause hepatotoxicity (including DILI) include alcohol, acetaminophen (paracetamol), NSAIDs (e.g., diclofenac and indometacin), glucocorticoids, hydrazine-containing drugs (e.g., isoniazid and iproniazid), antibiotics (e.g., amoxicillin, amoxicillin/clavulanic acid, and anti-tuberculosis drugs such as isoniazid, pyrazinamide and rifampicin), antiretrovirals (e.g., zidovudine [azidothymidine]), natural products (e.g., amanita mushrooms and green tea extract), alternative remedies (including herbal supplements and Chinese herbal remedies), and industrial toxins (e.g., arsenic, carbon tetrachloride and vinyl chloride). Acetaminophen followed by anti-tuberculosis drugs are the most common causes of ALF. Patterns of liver injury caused by chemicals (including medications) include zonal necrosis, hepatitis, cholestasis, steatosis, granulomas, vascular lesions and neoplasms. NRH, NARH or a reduced derivative thereof can be used to prevent hepatotoxicity (including DILI) in patients who are scheduled to take a medication (e.g., an anti-tuberculosis drug, an NSAID or a glucocorticoid) for an active disease (e.g., tuberculosis or an inflammatory disorder) or for a disease (e.g., tuberculosis or an inflammatory disorder) that is diagnosed (e.g., a positive tuberculosis test) but not yet active. NRH, NARH or a reduced derivative thereof enhances mitochondrial function and reduces oxidative stress, inflammation and cell death.

In other embodiments, NRH, NARH or a reduced derivative thereof (e.g., that of Formula I) is used in vivo or ex vivo to treat a hemolytic disorder. Hemolytic disorders include without limitation hemolysis and hemolytic anemia (hereditary/intrinsic causes and acquired/extrinsic causes). Anemia is a lower total amount of red blood cells (RBCs) or hemoglobin in the blood, or a diminished ability of the blood to carry oxygen. Hence, hemolysis (rupturing of RBCs) typically results in anemia, namely, hemolytic anemia. Therefore, the following intrinsic and extrinsic causes of hemolytic anemia also apply to hemolytic disorders more generally. Intrinsic causes of (hereditary) hemolytic anemia include without limitation defects in RBC membrane production or morphology (such as in hereditary spherocytosis, hereditary elliptocytosis and hereditary pyropoikilocytosis), defects in RBC metabolism (such as in cytochrome-b5 reductase deficiency, glucose-6-phosphate dehydrogenase [G6PD] deficiency, pyruvate kinase deficiency, 6-phosphogluconate dehydrogenase [6PGD] deficiency and gamma-glutamylcysteine synthetase deficiency), and defects in hemoglobin production (such as in cytochrome-b5 reductase deficiency, thalassemia, sickle cell disease, sickle cell anemia and congenital dyserythropoietic anemia). Extrinsic causes of (acquired) hemolytic anemia include without limitation attack of RBCs by the immune system (such as in autoimmune hemolytic anemia, SLE, rheumatoid arthritis, Hodgkin's lymphoma, chronic lymphocytic leukemia, cold agglutinin disease, Mycoplasma pneumoniae infection and paroxysmal nocturnal hemoglobinuria [PNH]), infections (e.g., malaria, Babesia, Clostridia, Haemophilus, Rickettsia, influenza and HIV), poisons and toxins (e.g., lead, arsine, stibine, toxins such as hemolysins and Shiga toxins produced by bacteria such as E. coli and Staphylococcus aureus, and toxins delivered via bites/stings of snakes and insects such as wasps and spiders), drugs/medications (e.g. adriamycin), radiation (e.g., radiation therapy for cancer), trauma (e.g., burns and trauma to RBCs caused by medical interventions such as endovascular devices, prosthetic heart valves and extracorporeal membrane oxygenation), microvascular angiopathies (e.g., hemolytic uremic syndrome, HELLP [hemolytic anemia, elevated liver enzymes, lactic acidosis and low platelets] syndrome, and thrombotic microangiopathies such as disseminated intravascular coagulation, thrombotic thrombocytopenic purpura, and those in SIRS and sepsis), other impairments of blood vessels (e.g., arteriovenous malformations, aortic stenosis, scleroderma and vasculitis), systemic disorders (e.g., SIRS, sepsis and malignant hypertension), impairment in cholesterol esterification (such as in spur-cell hemolytic anemia and CLD), and causes of increased spleen activity (e.g., splenomegaly and portal hypertension). Oxidative stress, damage or injury, such as that in or to RBCs, often induces, contributes to or exacerbates the hemolytic pathology of the aforementioned intrinsic and extrinsic causes of hemolytic anemia (or hemolytic disorders more generally).

In hemolysis, RBCs rupture and release their contents into surrounding fluid (e.g. blood plasma). Hemolysis can occur in blood vessels (intravascular hemolysis) or elsewhere in the body (extravascular hemolysis) such as in the spleen. Hemolysis can have serious consequences, including systemic inflammation, vasomotor dysfunction, thrombophilia, and acute or chronic kidney injury. Accumulation of potentially toxic, extracellular hemoproteins such as hemoglobin and degradation products thereof such as heme, iron and globin in the circulation resulting from hemolysis can cause hypertension and injure vascular tissues and the kidneys, which filter the plasma, via oxidative stress, inflammation and cytotoxicity/cell death mechanisms. The antioxidant, anti-inflammatory and anti-apoptotic properties of NRH, NARH and reduced derivatives thereof prevent or mitigate pathological effects of cell-free hemoglobin and degradation products thereof. Therefore, prevention or reduction of hemolysis by NRH, NARH and reduced derivatives thereof can prevent or ameliorate, e.g., hemolytic disorders, SIRS/sepsis, compromised hemodynamics such as in shock/septic shock, thrombo-embolic disorders (e.g., venous thrombo-embolism such as deep vein thrombosis in the legs and the arms [Paget-Schroetter disease], portal vein thrombosis, hepatic vein thrombosis, renal vein thrombosis, cerebral venous sinus thrombosis and pulmonary embolism), and AKI and CKD.

Surprisingly, NRH or a reduced derivative thereof, but not NR or an oxidized derivative thereof, exerted antioxidant and antihemolytic effects in an assay relating to oxidative damage to red blood cells (RBCs)/erythrocytes. Both NRH and NRHTA, but not NR or its oxidized derivative NR-triacetate (NRTA), reduced H₂O₂-induced hemolysis in vitro (Example 5). Oxidative stress damages RBCs and eventually results in their lysis (hemolysis), and hence reduces RBC production in the bone marrow and causes the death of RBCs in the circulation. See, e.g., E. Fibach and E. Rachmilewitz, Curr. Mol. Med., 8:609-619 (2008); and P. Maurya et al., World J. Methodol., 5:216-222 (2015). NRH and NRHTA can decrease oxidative stress in RBCs by, e.g., increasing NADH (reducing agent)/NAD (oxidizing agent) ratio and thereby improve cellular redox (reduction-oxidation) balance, decrease oxidative damage to RBCs, and prevent or decrease hemolysis. The antioxidant and antihemolytic effects of NRH and NRHTA in RBCs are mitochondria-independent because mature RBCs lack mitochondria.

In further embodiments, NRH, NARH or a reduced derivative thereof (e.g., that of Formula I) is used in vivo or ex vivo to treat a disorder or condition associated with oxidative stress, damage or injury. Disorders and conditions associated with oxidative stress, damage or injury, whether oxidative stress, damage or injury is a cause of the disorder or condition or/and a result of the disorder or condition that contributes to its pathological effects, include without limitation oxidative stress in or oxidative damage or injury to cells, tissues or organs induced by endogenous processes (e.g., generation of reactive oxygen and nitrogen species by activated immune cells such as neutrophils and macrophages and by enzymes such as oxidases [e.g., NADPH oxidase and xanthine oxidase], oxygenases [e.g., mono-oxygenases and dioxygenases], and nitric oxide synthases), infections (e.g., with viruses such as hepatitis B, C and D viruses, HIV, Epstein-Barr virus, influenza A and respiratory syncytial virus, or with bacteria such as Helicobacter pylori and Bacteroides fragilis), drugs/medications (infra), plant alkaloids (e.g., berberine and sanguinarine), metabolites (e.g., chloroacetaldehyde [a toxic metabolite of ifosfamide] and metabolites of atorvastatin), foods (e.g., broad/fava bean), toxins and poisons (e.g., alcohol, carbon tetrachloride, 1-methyl-4-phenyl-1,2,3,6-tetrahydropyridine [MPTP] and iron) or radiation (e.g., UV and ionizing radiation such as X-ray); oxidative stress in or oxidative damage or injury to red blood cells (RBCs), white blood cells (WBCs, including lymphocytes such as T cells, B cells and natural killer cells), kidney cells (e.g., renal tubular epithelial cells), hepatocytes, lung cells (e.g., lung epithelial cells and alveolar cells), sperm cells and oocytes; blood disorders; disorders involving defective metabolism of minerals (e.g., copper-overload disorders such as Wilson's disease, and iron-overload disorders such as hemochromatosis (e.g., hereditary hemochromatosis and secondary hemochromatosis [e.g., transfusional iron overload and iron overload secondary to a liver disorder such as ALD, NAFLD, ASH, NASH, or alcoholic or non-alcoholic cirrhosis]), aceruloplasminemia, pantothenate kinase-associated neurodegeneration (Hallervorden-Spatz syndrome) and Friedreich's ataxia}; oxidative damage or injury to tissues and organs (e.g., the liver and kidney) having high levels of metalloproteins (e.g., hemoglobin, myoglobin, cytochromes [e.g., cytochromes a, b, c and d], proteins with an iron-sulfur cluster, ferritin and Cu—Zn superoxide dismutase); encephalopathy induced by drugs/medications (e.g., chemotherapeutics such as ifosfamide); airway disorders (e.g., asthma, COPD, acute and chronic lung injury, and ARDS); eye disorders (e.g., cataract, maculopathy [e.g., AMD], and retinopathy [e.g., non-proliferative retinopathy and retinal degeneration]); male and female infertility; immune-related disorders; kidney disorders; liver disorders; and hemolytic disorders.

By preventing or decreasing oxidative stress in cells, NRH, NARH and reduced derivatives thereof can also prevent or decrease undesired oxidation of or/and oxidative damage to components thereof, such as proteins, lipids, DNA, organelles and other subcellular compartments. For example, NRH, NARH and reduced derivatives thereof can be used in vivo or ex vivo to prevent or decrease oxidation of metalloproteins, such as oxidation of hemoglobin to methemoglobin. The hemoglobin can be the predominant form of normal hemoglobin, hemoglobin A (HbA), or a hemoglobin variant (e.g., HbH, HbS or Hb-Barts) present in diseased states (e.g., sickle cell disease and thalassemia). The iron in the heme group of methemoglobin is in the Fe³⁺ (ferric) state, not the Fe²⁺ (ferrous) state of normal hemoglobin, and thus cannot bind oxygen and transport oxygen to tissues and organs.

Drugs/medications that can induce oxidative stress, damage or injury include without limitation iron preparations, methylene blue, L-dopa, NSAIDs (e.g., diclofenac and indometacin), calcineurin inhibitors (e.g., cyclosporin and tacrolimus), antipyretics (e.g., paracetamol [acetaminophen]), antipsychotics (e.g., clozapine and phenothiazine derivatives such as chlorpromazine), selective estrogen receptor modulators (e.g., tamoxifen), anticancer drugs (e.g., actinomycin D, bleomycin, camptothecin, carmofur, cisplatin, doxorubicin, gemcitabine, mercaptopurine, mitomycin C, mitoxantrone, nimustine, paclitaxel, vinblastine and vinorelbine), antibiotics (e.g., gentamicin), and antiretrovirals (e.g., zidovudine [azidothymidine]).

Blood disorders associated with oxidative stress, damage or injury include without limitation methemoglobinemia (acquired and genetic), anemia (e.g., congenital dyserythropoietic anemia, hemolytic anemia, sickle cell anemia, G6PD deficiency-related anemia and PNH), thalassemia (including alpha-, beta- and delta-thalassemia), and abnormal morphology or shape of RBCs (e.g., hereditary elliptocytosis/ovalocytosis, hereditary spherocytosis and sickle cell disease). In certain embodiments, the blood disorders are methemoglobinemia (acquired or genetic) and anemia (due to, e.g., hemolysis, an increased methemoglobin/hemoglobin ratio or methemoglobinemia).

Methemoglobinemia is elevated methemoglobin level in the blood. It may have serious complications such as seizures and heart arrhythmias. It may be acquired or genetic. Methemoglobinemia can be induced by, e.g., dialysis, drugs/medications (e.g., antibiotics [e.g., dapsone, sulfonamides and trimethoprim], local anesthetics [e.g., articaine, benzocaine, lidocaine and prilocaine], methylene blue, metoclopramide and rasburicase), chemical compounds (e.g., aniline dyes, bromates, chlorates, nitrates and nitrites), and foods (e.g., broad/fava bean). Genetic causes of methemoglobinemia include without limitation abnormal hemoglobin variants (e.g., hemoglobin H and hemoglobin M), deficiency in cytochrome-b5 reductase (methemoglobin reductase) that reduces methemoglobin to hemoglobin, and deficiency in pyruvate kinase or G6PD involved in production of the NADH or NADPH cofactor for cytochrome-b5 reductase. Methemoglobinemia can induce an inflammatory response, and can worsen oxygenation in SIRS or sepsis. See, e.g., Anna et al., Am. J. Physiol. Lung Cell. Mol. Physiol., 294:L161-L174 (2008); and Jay et al., Am. J. Hematol., 82:134-144 (2007).

Anemia is a lower total amount of RBCs or hemoglobin in the blood, or a diminished ability of the blood to carry oxygen. Anemia can be due to, e.g., blood loss (caused by, e.g, trauma or gastrointestinal bleeding), reduced RBC production (caused by, e.g., dyserythropoiesis, iron deficiency, vitamin B₉ [folate] deficiency, vitamin B₁₂ [cobalamin] deficiency, thalassemia, certain neoplasms of the bone marrow such as myelodysplastic syndrome, myelosuppressive drugs such as zidovudine, or certain infections such as HIV infection), increased RBC breakdown (caused by, e.g., dyserythropoiesis, hemolysis [hemolytic anemia], sickle cell anemia, hereditary pyropoikilocytosis, G6PD deficiency, pyruvate kinase deficiency, PNH, certain infectious disorders such as malaria, or certain autoimmune disorders such as SLE and rheumatoid arthritis), reduced hemoglobin production (caused by, e.g., thalassemia or cytochrome-b5 reductase deficiency), an increased methemoglobin/hemoglobin ratio, or methemoglobinemia.

In some embodiments, NRH, NARH or a reduced derivative thereof (e.g., that of Formula I) is used in vivo or ex vivo to prevent or decrease oxidative stress in or oxidative damage or injury to RBCs, hemolysis or formation of methemoglobin, or any combination or all thereof. In certain embodiments, the subject suffers from an immune-related disorder such as SIRS or sepsis or a complication thereof, from a hemolytic disorder or from a blood disorder such as anemia. In other embodiments, the subject is undergoing hemodialysis or hemofiltration and has any underlying disorder, such as SIRS, sepsis, AKI, CKD, ESKD, liver failure, HRS or MODS.

In further embodiments, NRH, NARH or a reduced derivative thereof (e.g., that of Formula I) is used in vivo or ex vivo as an alternative to methylene blue or as an adjuvant to methylene blue for enhancing the safety or/and efficacy of methylene blue, to treat a disorder or condition for which methylene blue may be indicated or contra-indicated, such as methemoglobinemia, ifosfamide-induced encephalopathy, sepsis, septic shock or anaphylaxis. Excessive doses of methylene blue can induce oxidative stress and methemoglobinemia itself. Similarly, NRH, NARH or a reduced derivative thereof (e.g., that of Formula I) can be used in vivo or ex vivo as an adjuvant to other drugs or radiation therapies that (or metabolites of the drugs) induce oxidative stress in or oxidative damage or injury to cells, tissues or organs to enhance the safety or/and efficacy of the drugs or radiation therapies. For example, NRH, NARH or a reduced derivative thereof can be used in vivo or ex vivo to enhance the safety or/and efficacy of iron preparations (e.g., intravenous ones) that induce oxidative stress in patients undergoing iron-enhancing therapy. Also similarly, NRH, NARH or a reduced derivative thereof (e.g., that of Formula I) can be used in vivo or ex vivo as an adjuvant to other drugs or therapies to prevent or decrease oxidative stress in or oxidative damage or injury to cells, tissues or organs.

In other embodiments, NRH, NARH or a reduced derivative thereof (e.g., that of Formula I) is used to treat an oxidative stress-associated eye disorder. In certain embodiments, the eye disorder is cataract. Antioxidant effects of NRH, NARH or a reduced derivative thereof in the lens, such as enhancement of cellular redox balance in the lens, can prevent or reduce oxidative damage to the lens and thereby prevent or delay the formation of cataract, or slow the progression or reduce the severity of cataract. In certain embodiments, NRH, NARH or a reduced derivative thereof is administered to the eye by eye drop.

NRH, NARH and reduced derivatives thereof (e.g., those of Formula I) can also have in vitro, or other ex vivo, applications. In some embodiments, a biological sample (e.g., cells or biological fluid) from a subject is contacted with NRH, NARH or a reduced derivative in vitro. For example, blood and blood products (e.g., packed RBCs) can be treated with NRH, NARH or a reduced derivative thereof in vitro to increase the quality and shelf-life thereof by preventing or decreasing oxidative stress therein, oxidative damage or injury thereto, and hemolysis thereof during storage. Such treated blood and blood products can be used in autologous or heterologous blood transfusion for subjects with, e.g., SIRS or sepsis or a complication thereof such as shock/septic shock, with a hemolytic disorder or with a blood disorder such as anemia. As another example, sperm cells or semen can be treated with NRH, NARH or a reduced derivative thereof in vitro to treat male infertility for any reason(s), such as to enhance the health, function, motility or number of sperm cells, or any combination or all thereof. As an additional example, oocytes or follicular fluid can be treated with NRH, NARH or a reduced derivative thereof in vitro to treat female infertility for any reason(s), such as to enhance the health, function or number of oocytes, or any combination or all thereof.

The dose or therapeutically effective amount and the frequency of administration of, and the length of treatment with, NRH, NARH or a reduced derivative thereof (e.g., that of Formula I) to treat a disorder or condition disclosed herein in vivo or ex vivo may depend on various factors, including the nature and severity of the disorder or condition, the potency of the compound, the route of administration, the age, body weight, general health, gender and diet of the subject, and the response of the subject to the treatment, and can be determined by the treating physician. In some embodiments, the dose or therapeutically effective amount of NRH, NARH or a reduced derivative thereof (e.g., that of Formula I) to treat a disorder or condition disclosed herein is about 0.1-60 mg/kg, 0.5-50 mg/kg, 1-40 mg/kg or 1.5-30 mg/kg per day, or about 1-4000 mg, 50-3500 mg, 100-3000 mg or 100-2000 mg per day, or as deemed appropriate by the treating physician, which can be administered, e.g., as a bolus in a single dose (e.g., N mg once daily) or multiple doses (e.g., N/2 mg twice daily) or by continuous infusion. In further embodiments, the dose or therapeutically effective amount of NRH, NARH or a reduced derivative thereof is about 1 mg-1 g, 1-2 g, 2-3 g or 3-4 g per day. In still further embodiments, the dose or therapeutically effective amount of NRH, NARH or a reduced derivative thereof is about 1-500 mg or 500-1000 mg per day, or about 1-50 mg, 50-100 mg, 100-200 mg, 200-300 mg, 300-400 mg, 400-500 mg, 500-750 mg or 750-1000 mg per day. In additional embodiments, the dose or therapeutically effective amount of NRH, NARH or a reduced derivative thereof is about 50 mg, 100 mg, 150 mg, 200 mg, 250 mg, 300 mg, 400 mg, 500 mg, 600 mg, 700 mg, 800 mg, 900 mg or 1000 mg per day. In certain embodiments, the dose or therapeutically effective amount of NRH, NARH or a reduced derivative thereof is about 100-500 mg, 100-200 mg, 200-300 mg, 300-400 mg or 400-500 mg per day, or about 100 mg, 150 mg, 200 mg, 250 mg, 300 mg, 350 mg, 400 mg, 450 mg or 500 mg per day.

The dose or therapeutically effective amount of NRH, NARH or a reduced derivative thereof (e.g., that of Formula I) can be administered to a patient or provided ex vivo in any suitable frequency, and can be determined by the treating physician. In some embodiments, NRH, NARH or a reduced derivative thereof is administered to a patient or provided ex vivo one, two or more (e.g., three or four) times a day, once every two days, once every three days, thrice a week, twice a week or once a week. In certain embodiments, the dose or therapeutically effective amount of NRH, NARH or a reduced derivative thereof is administered to a patient once or twice daily. As an illustrative example, if the dose or therapeutically effective amount of NRH, NARH or a reduced derivative thereof is about 500 mg per day, 500 mg of the compound can be administered once daily, or 250 mg of the compound can be administered twice daily.

Where a more rapid establishment of a therapeutic level of NRH, NARH or a reduced derivative thereof (e.g., that of Formula I) is desired, such as in the treatment of SIRS, sepsis, an ischemia-reperfusion injury or an acute disorder, the compound can be administered under a dosing schedule in which a loading dose is administered, followed by (i) one or more additional loading doses and then one or more therapeutically effective maintenance doses, or (ii) one or more therapeutically effective maintenance doses without an additional loading dose, as deemed appropriate by the treating physician. In such a case, a loading dose of a drug is larger (e.g., about 1.5, 2, 3, 4 or 5 times larger) than a subsequent maintenance dose and is designed to establish a therapeutic level of the drug more quickly. The one or more therapeutically effective maintenance doses can be any dose or therapeutically effective amount described herein. In certain embodiments, the loading dose is about three times larger than the maintenance dose. In some embodiments, a loading dose of NRH, NARH or a reduced derivative thereof is administered on day 1 and a maintenance dose is administered on day 2 and thereafter for the duration of therapy. In other embodiments, a first loading dose of NRH, NARH or a reduced derivative thereof is administered on day 1, a second loading dose is administered on day 2, and a maintenance dose is administered on day 3 and thereafter for the duration of therapy. In certain embodiments, the first loading dose is about three times larger than the maintenance dose, and the second loading dose is about two times larger than the maintenance dose.

The length of in vivo or ex vivo treatment with NRH, NARH or a reduced derivative thereof (e.g., that of Formula I) can be based on, e.g., the nature and severity of the disorder or condition and the response of the subject to the treatment, and can be determined by the treating physician. In certain embodiments, a dose or therapeutically effective amount of NRH, NARH or a reduced derivative thereof is administered to a patient or provided ex vivo over a period of about 1, 2, 3, 4, 5 or 6 days, or about 1, 2, 3, 4, 5 or 6 weeks, to treat an acute disorder or condition. Acute disorders and conditions include without limitation SIRS and sepsis, damage and injury to tissues and organs (e.g., the brain, spinal cord, kidney and liver), ischemic disorders (e.g., myocardial ischemia/infarction and cerebral ischemia/infarction), and ischemia-reperfusion injury (e.g., cardiac IRI, cerebral IRI and renal IRI). In other embodiments, a dose or therapeutically effective amount of NRH, NARH or a reduced derivative thereof is administered to a patient or provided ex vivo over a period of at least about 6 weeks, 8 weeks (2 months), 3 months, 6 months, 1 year, 2 years, 3 years, 4 years, 5 years, 10 years or longer to treat a chronic disorder or condition. It is understood that the delineation between acute and chronic may vary based on, e.g., the particular disorder or condition. For example, a particular disorder may be deemed acute by specialists in that discipline if it endures up to 6 weeks, while another disorder may be deemed acute by specialists in that discipline if it endures up to 8 weeks. It is further understood that the duration of a particular disorder or condition in individual patients often varies and may be acute or chronic.

NRH, NARH or a reduced derivative thereof (e.g., that of Formula I) can also be used in vivo or ex vivo pro re nata (as needed) until clinical manifestations of the disorder or condition disappear or clinical targets for that disorder or condition are achieved. If clinical manifestations of the disorder or condition re-appear or the clinical targets are not maintained, in vivo or ex vivo use of NRH, NARH or a reduced derivative thereof can resume. Under an alternative in vivo or ex vivo pro re nata treatment and also at the treating physician's discretion, the dose of NRH, NARH or a reduced derivative thereof or/and its dosing frequency can be reduced upon improvement of clinical target(s) or outcome(s) and then can be increased (e.g., to the previously effective dose or/and dosing frequency) if the patient's clinical status subsequently worsens.

For in vivo use, NRH, NARH and reduced derivatives thereof (e.g., those of Formula I) can be administered to a patient via any suitable route. Potential routes of administration of NRH, NARH and reduced derivatives thereof include without limitation oral, parenteral (including intradermal, subcutaneous, intravascular, intravenous, intra-arterial, intramuscular, intraperitoneal, intracavitary, intramedullary, intrathecal and topical), and topical (including dermal/epicutaneous, transdermal, mucosal, transmucosal, intranasal [e.g., by nasal spray or drop], pulmonary [e.g., by oral or nasal inhalation], ocular [e.g., by eye drop], buccal, sublingual, rectal [e.g., by suppository] and vaginal [e.g., by suppository]). In some embodiments, NRH, NARH or a reduced derivative thereof is administered orally, e.g., as a tablet or capsule that optionally has an enteric coating (e.g., Opadry© Enteric [94 Series]). In other embodiments, NRH, NARH or a reduced derivative thereof is administered (e.g., by injection or infusion) parenterally, such as intravenously, subcutaneously or intramuscularly.

The route of administration can depend on, e.g., the particular disorder or condition being treated. As an example, for treatment of an eye disorder (e.g., cataract), NRH, NARH or a reduced derivative thereof can be administered, e.g., by eye drop. As another example, for treatment of a skin disorder or condition, a topical composition containing NRH, NARH or a reduced derivative thereof can be applied to the affected area(s) of the skin. As an additional example, for treatment of an airway disorder, NRH, NARH or a reduced derivative thereof can be administered by oral inhalation.

In some embodiments, contacting cells or biological fluid from a subject ex vivo comprises contacting blood, plasma, serum, lymphatic fluid, cerebrospinal fluid, synovial fluid, semen or follicular fluid from the subject ex vivo with NRH, NARH or a reduced derivative thereof (e.g., that of Formula I). In certain embodiments, the subject suffers from an immune-related disorder (e.g., SIRS or sepsis), a hemolytic disorder (e.g., hemolysis) or a blood disorder (e.g., anemia), and blood from the subject is treated ex vivo with NRH, NARH or a reduced derivative thereof.

In some embodiments, the concentration (e.g., steady-state concentration) of NRH, NARH or a reduced derivative thereof (e.g., that of Formula I) in the blood after administration to a patient or in an ex vivo medium (e.g., blood) is about 1-1000 μM, 1-500 μM or 500-1000 μM, or about 1-250 μM, 250-500 μM, 500-750 μM or 750-1000 μM. In certain embodiments, the concentration (e.g., steady-state concentration) of NRH, NARH or a reduced derivative thereof in the blood after administration to a patient or in an ex vivo medium (e.g., blood) is about 1-200 μM, 1-150 μM, 1-100 μM or 100-200 μM, or about 1-50 μM, 50-100 μM, 100-150 μM or 150-200 μM. In some embodiments, the concentration (e.g., steady-state concentration) of NRH, NARH or a reduced derivative thereof in the blood after administration to a patient or in an ex vivo medium (e.g., blood) persists for at least about 1 hr, 2 hr, 3 hr, 6 hr, 8 hr or 12 hr per administration or treatment. In some embodiments, NRH, NARH or a reduced derivative thereof is intravenously or subcutaneously administered to a patient as a bolus one, two, three or four times daily, or by continuous infusion.

For in vivo use, NRH, NARH or a reduced derivative thereof (e.g., that of Formula I) can be administered at any time convenient to the patient, such as in the morning or/and at nighttime (e.g., bedtime). Moreover, NRH, NARH or a reduced derivative thereof (e.g., that of Formula I) can be taken substantially with food (e.g., with a meal or within about 1 hour or 30 minutes before or after a meal) or substantially without food (e.g., at least about 1 or 2 hours before or after a meal).

The disclosure provides a method of treating a disorder or condition described herein, comprising administering to a subject in need of treatment a therapeutically effective amount of, or contacting cells or biological fluid from a subject in need of treatment ex vivo with, dihydronicotinamide riboside (NRH), dihydronicotinic acid riboside (NARH) or a reduced derivative thereof (e.g., that of Formula I), or a pharmaceutically acceptable salt, solvate, hydrate, clathrate, polymorph or stereoisomer thereof, or a pharmaceutical composition comprising the same. The disclosure further provides NRH, NARH or a reduced derivative thereof (e.g., that of Formula I), or a pharmaceutically acceptable salt, solvate, hydrate, clathrate, polymorph or stereoisomer thereof, or a composition comprising the same, for use in the treatment of a disorder or condition described herein. In addition, the disclosure provides for the use of NRH, NARH or a reduced derivative thereof (e.g., that of Formula I), or a pharmaceutically acceptable salt, solvate, hydrate, clathrate, polymorph or stereoisomer thereof, in the manufacture or preparation of a medicament for the treatment of a disorder or condition described herein. In some embodiments, the disorder or condition is an immune-related disorder (e.g., SIRS or sepsis), a kidney disorder (e.g., AKI or HRS), a liver disorder (e.g., alcoholic hepatitis, ALF, ACLF, cirrhosis or HRS), a hemolytic disorder (e.g., hemolysis or hemolytic anemia), or a disorder or condition associated with oxidative stress, damage or injury (e.g., methemoglobinemia or anemia). NRH, NARH or a reduced derivative thereof can be used in vivo or ex vivo alone or in combination with one or more additional therapeutic agents (e.g., an anti-inflammatory agent or/and an antioxidant).

In some embodiments, NRH, NARH, NRH-triacetate (NRHTA), NARH-triacetate (NARHTA), or a pharmaceutically acceptable salt thereof is used in vivo or ex vivo to treat a disorder or condition described herein (e.g., an immune-related disorder, a kidney disorder, a liver disorder, a hemolytic disorder, or a disorder or condition associated with oxidative stress, damage or injury), or in in vitro applications.

The description and all of the embodiments relating to therapeutic uses of NRH, NARH and reduced derivatives thereof also apply to therapeutic uses of metabolites of NRH, NARH and reduced derivatives thereof and to therapeutic uses of intermediates in the biosynthesis of NADH from NRH or NARH, such as NMNH and NAMNH. Nicotinamide/nicotinic acid riboside kinase (NRK) phosphorylates NRH to dihydronicotinamide mononucleotide (NMNH), which is then converted to dihydronicotinamide adenine dinucleotide (NADH) by nicotinamide/nicotinic acid mononucleotide adenyltransferase (NMNAT). Similarly, NRK phosphorylates NARH to dihydronicotinic acid mononucleotide (NAMNH), which is converted to dihydronicotinic acid adenine dinucleotide (NAADH) by NMNAT, which in turn is converted to NADH by NAD⁺ synthetase 1 (NADSYN1).

Combination Therapies with Other Therapeutic Agents

NRH, NARH or a reduced derivative thereof (e.g., that of Formula I [infra]) can be used in vivo or ex vivo alone or in combination with one or more additional therapeutic agents to treat any disorder or condition disclosed herein. For in vivo or ex vivo use, the additional therapeutic agent(s) can be administered or provided prior to, concurrently with or subsequent to administration or provision of NRH, NARH or a reduced derivative thereof. For in vivo or ex vivo use, the additional therapeutic agent(s) and NRH, NARH or a reduced derivative thereof can be administered or provided in the same pharmaceutical composition or in separate compositions.

In some embodiments, NRH, NARH or a reduced derivative thereof (e.g., that of Formula I) is used in vivo or ex vivo in combination with an anti-inflammatory agent to treat any disorder or condition disclosed herein. In some embodiments, the disorder or condition is an immune-related disorder. In certain embodiments, the immune-related disorder is SIRS or sepsis, or a complication thereof. In some embodiments, the anti-inflammatory agent is or comprises an NSAID, a glucocorticoid, an immunosuppressant, or an inhibitor of pro-inflammatory cytokine(s) or receptor(s) therefor or the production thereof (e.g., TNF-α, IL-2, IL-4, IL-6 or IL-23, or any combination thereof), or any combination or all thereof.

Anti-inflammatory agents include without limitation:

non-steroidal anti-inflammatory drugs (NSAIDs), including those listed below;

immunomodulators, including imides (e.g., thalidomide, lenalidomide, pomalidomide and apremilast) and xanthine derivatives (e.g., lisofylline, pentoxifylline and propentofylline); immunosuppressants, including interferon-beta (IFN-β), cyclophosphanide, glucocorticoids (infra), antimetabolites (e.g., hydroxyurea [hydroxycarbamide], antifolates [e.g., methotrexate], and purine analogs [e.g., azathioprine, mercaptopurine and thioguanine]), pyrimidine synthesis inhibitors (e.g., leflunomide and teriflunomide), calcineurin inhibitors (e.g., ciclosporin [cyclosporine A], pimecrolimus and tacrolimus), inosine-5′-monophosphate dehydrogenase (IMPDH) inhibitors (e.g., mycophenolic acid and derivatives thereof [e.g., mycophenolate sodium and mycophenolate mofetil]), mechanistic/mammalian target of rapamycin (mTOR) inhibitors (e.g., rapamycin [sirolimus], deforolimus [ridaforolimus], everolimus, temsirolimus, umirolimus [biolimus A9], zotarolimus and RTP-801), modulators of sphingosine-1-phosphate receptors (e.g., S1PR1) (e.g., fingolimod), and serine C-palmitoyltransferase inhibitors (e.g., myriocin); anti-inflammatory cytokines and compounds that increase their production, including IL-10 and analogs and derivatives thereof (e.g., PEG-ilodecakin) and compounds that increase IL-10 production {e.g., S-adenosyl-L-methionine, melatonin, metformin, rotenone, curcuminoids (e.g., curcumin), triterpenoids (e.g., oleanolic acid analogs [infra, such as TP-225]), prostacyclin and analogs thereof (infra), and apoA-I mimetics (e.g., 4F)}; inhibitors of pro-inflammatory cytokines or receptors therefor, including inhibitors of (e.g., antibodies or fragments thereof targeting) tumor necrosis factor-alpha (TNF-α) (e.g., adalimumab, certolizumab pegol, golimumab, infliximab, etanercept, bupropion, curcunin, catechins and ART-621) or the receptor therefor (TNFR1), inhibitors of thymic stromal lymphopoietin (e.g., anti-TSLP antibodies and fragments thereof [e.g., tezepelumab and M702] and immunoconjugates comprising the extracellular domain of TSLPR) or the receptor therefor (TSLPR), inhibitors of (e.g., antibodies or fragments thereof targeting) pro-inflammatory interferons (e.g., interferon-alpha [IFN-α]) or receptors therefor, inhibitors of (e.g., antibodies or fragments thereof targeting) pro-inflammatory interleukins or receptors therefor {e.g., IL-1 (e.g., IL-1α and IL-1β [e.g., canakinumab and rilonacept]) or IL-1R (e.g., anakinra and isunakinra [EBI-005]), IL-2 or IL-2R (e.g., basiliximab and daclizumab), IL-4 or IL-4R (e.g., dupilumab), IL-5 (e.g., mepolizumab and reslizumab) or IL-5R, IL-6 (e.g., clazakizumab, elsilimomab, olokizumab, siltuximab and sirukumab) or IL-6R (e.g., sarilumab and tocilizumab), IL-8 or IL-8R, IL-12 (e.g., briakinumab and ustekinumab) or IL-12R, IL-13 or IL-13R, IL-15 or IL-15R, IL-17 (e.g., ixekizumab and secukinumab) or IL-17R (e.g., brodalumab), IL-18 (e.g., GSK1070806) or IL-18R, IL-20 (e.g., the antibody 7E) or IL-20R, IL-22 (e.g., fezakinumab) or IL-22R, IL-23 (e.g., briakinumab, guselkumab, risankizuniab, tildrakizumab [SCH-900222], ustekinumab and BI-655066) or IL-23R, IL-31 (e.g., anti-IL-31 antibodies disclosed in U.S. Pat. No. 9,822,177) or IL-31R (e.g., anti-IL-31 receptor A antibodies such as nemolizumab), IL-33 or IL-33R, and IL-36 or IL-36R}, and inhibitors of monocyte chemoattractant protein 1 (MCP-1) {e.g., bindarit, anti-MCP1 antibodies (e.g., 5D3-F7 and 10F7), MCP1-binding peptides (e.g., HSWRHFHTLGGG), and MCP1-binding RNA aptamers (e.g., ADR22 and mNOX-E36 [a spiegelmer])} or receptors therefor (e.g., CCR2 antagonists such as spiropiperidines [e.g., RS-29634, RS-102895 and RS-504393]); inhibitors of the production of pro-inflammatory cytokines or receptors therefor, including inhibitors of the production of TNF-α {e.g., N-acetyl-L-cysteine, S-adenosyl-L-methionine, L-carnitine, hydroxychloroquine, melatonin, parthenolide, pirfenidone, sulfasalazine, mesalazine (5-aminosalicylic acid), taurine, flavonoids (e.g., epigallocatechin-3-gallate [EGCG], naringenin and quercetin), omega-3 fatty acids and esters thereof [infra], glucocorticoids, immunomodulatory imides and xanthine derivatives, PDE4 inhibitors [infra], serine protease inhibitors (e.g., gabexate and nafamostat), prostacyclin and analogs thereof, SOCS1 mimetics (infra) myxoma virus M013 protein, Yersinia YopM protein, apoA-I mimetics (e.g., 4F), and apoE mimetics (e.g., AEM-28 and hEp)}, IFN-α (e.g., alefacept), IL-1 (e.g., IL-1α, and IL-1β) (e.g., chloroquine, hydroxychloroquine, nafamostat, pirfenidone, sulfasalazine, mesalazine, prostacyclin and analogs thereof, glucocorticoids, TNF-α inhibitors, PAR1 antagonists [e.g., vorapaxar], M013 protein, YopM protein and apoA-I mimetics [e.g., 4F]), IL-1β (e.g. melatonin, metformin, rotenone, flavonoids [e.g., EGCG and naringenin], annexin A1 mimetics, and caspase-1 inhibitors [e.g., belnacasan, pralnacasan and parthenolide]), IL-2 (e.g., glucocorticoids, calcineurin inhibitors and PDE4 inhibitors), IL-4 (e.g., glucocorticoids and serine protease inhibitors [e.g., gabexate and nafamostat]), IL-5 (e.g., glucocorticoids), IL-6 (e.g., nafamostat, parthenolide, prostacyclin and analogs thereof, tranilast, L-carnitine, taurine, flavonoids [e.g., EGCG, naringenin and quercetin], omega-3 fatty acids and esters thereof, glucocorticoids, immunomodulatory imides, TNF-α inhibitors, M013 protein and apoE mimetics [e.g., AEM-28 and hEp]), IL-8 (e.g., alefacept and glucocorticoids), IL-12 (e.g., apilimod, PDE4 inhibitors and YopM protein), IL-15 (e.g., YopM protein), IL-17 (e.g., protein kinase C inhibitors such as sotrastaurin), IL-18 (e.g., M013 protein, YopM protein and caspase-1 inhibitors), IL-23 (e.g., apilimod, alefacept and PDE4 inhibitors), and MCP-1 (e.g., EGCG, melatonin and tranilast); inhibitors of pro-inflammatory transcription factors or their activation or expression, including inhibitors of NF-κB or its activation or expression {e.g., aliskiren, melatonin, minocycline and parthenolide (both inhibit NF-κB nuclear translocation), nafamostat, niclosamide, (−)-DHMEQ, IT-603, IT-901, PBS-1086, flavonoids (e.g., EGCG and quercetin), hydroxycinnamic acids and esters thereof (e.g., ethyl caffeate), lipoxins (e.g., 15-epi-LXA4 and LXB4), omega-3 fatty acids and esters thereof, stilbenoids (e.g., resveratrol), statins (e.g., rosuvastatin), triterpenoids (e.g., oleanolic acid analogs such as TP-225), TNF-α inhibitors, apoE mimetics (e.g., AEM-28), M013 protein, penetratin, and activators of sirtuin 1 (SIRT1, which inhibits NF-κB) (e.g., flavones [e.g., luteolin], phenylethanoids [e.g., tyrosol, which induces SIRT1 expression], stilbenoids [e.g., resveratrol, which increases SIRT1 activity and expression] and lamin A)}, and inhibitors of STAT (signal transducer and activator of transcription) proteins or their activation or expression {e.g., Janus kinase 1 (JAK1) inhibitors (e.g., itacitinib, upadacitinib, GLPG0634 and GSK2586184), JAK2 inhibitors (e.g., lestaurtinib, pacritinib, CYT387, TG101348, SOCS1 mimetics and SOCS3 mimetics), JAK3 inhibitors (e.g., ASP-015K, R348 and VX-509), dual JAK1/JAK2 inhibitors (e.g., baricitinib and ruxolitinib), dual JAK1/JAK3 inhibitors (e.g., tofacitinib), suppressor of cytokine signaling (SOCS) mimetic peptides (e.g., SOCS1 mimetics [e.g., SOCS1-KIR, NewSOCS1-KIR, PS-5 and Tkip] and SOCS3 mimetics), niclosamide, hydroxycinnamic acids and esters thereof (e.g., rosmarinic acid), and lipoxins (e.g., 15-epi-LXA4 and LXB4)}; inhibitors of pro-inflammatory prostaglandins (e.g., prostaglandin E₂ [PGE₂]) or receptors therefor (e.g., EP₃) or the production thereof, including cyclooxygenase inhibitors (e.g., NSAIDs [including non-selective COX-1/COX-2 inhibitors such as aspirin and selective COX-2 inhibitors such as coxibs], glucocorticoids [which inhibit COX activity and expression], omega-3 fatty acids and esters thereof, curcuminoids [e.g., curcumin], stilbenoids [e.g., resveratrol, which inhibits COX-1 and -2 activity and expression], and vitamin E and analogs thereof [e.g., α-tocopherol and trolox]), cyclopentenone prostaglandins (e.g., prostaglandin J₂ [PGJ₂], Δ12-PGJ₂ and 15-deoxy-Δ12,14-PGJ₂), hydroxycinnamic acids and esters thereof (e.g., ethyl caffeate, which suppresses COX-2 expression), and triterpenoids (e.g., oleanolic acid analogs such as TP-225, which suppress COX-2 expression); inhibitors of leukotrienes or receptors therefor or the production thereof, including cysteinyl leukotriene receptor 1 (cysLTR1) antagonists (e.g., cinalukast, gemilukast [dual cysLTR1/cysLTR2 antagonist], iralukast, montelukast, pranlukast, tomelukast, verlukast, zafirlukast, CP-195494, CP-199330, ICI-198615, MK-571 and lipoxins [e.g., LXA4 and 15-epi-LXA4]), cysLTR2 antagonists (e.g., HAMI-3379), 5-ipoxygenase (5-LOX) inhibitors (e.g., baicalein, caffeic acid, curcuimin, hyperforin, γ-inolenic acid [GLA], meclofenamic acid, meclofenamate sodium, minocycline, tipelukast [MN-001], zileuton, MK-886, and omega-3 fatty acids and esters thereof), and immunomodulatory xanthine derivatives; inhibitors of phospholipase A2 (e.g., secreted and cytosolic PLA2), including glucocorticoids, arachidonyl trifluoromethyl ketone, bromoenol lactone, chloroquine, cytidine 5-diphosphoamines, darapladib, quinacrine, vitamin E, RO-061606, ZPL-521, lipocortins (annexins, such as annexin A1), and annexin mimetic peptides (e.g., annexin A1 mimetics such as Ac2-26 and CGEN-855A); suppressors of C-reactive protein (CRP) activity or level, including statins (e.g., rosuvastatin), thiazolidinediones (e.g., balaglitazone, ciglitazone, darglitazone, englitazone, lobeglitazone, netoglitazone, pioglitazone, deuterated (R)-pioglitazone [e.g., DRX-065], rivoglitazone, rosiglitazone and troglitazone), dipeptidyl peptidase 4 (DPP-4) inhibitors (e.g., alogliptin, anagliptin, dutogliptin, evogliptin, gemigliptin, gosogliptin, linagliptin, omarigliptin, saxagliptin, septagliptin, sitagliptin, des-fluoro-sitagliptin, teneligliptin, trelagliptin and vildagliptin), stilbenoids (e.g., resveratrol), EGCG and CRP-i2; mast cell stabilizers, including cromoglicic acid (cromolyn), ketotifen, methylxanthines, nedocromil, nicotinamide, olopatadine, omalizumab, pemirolast, quercetin, zinc sulfate, and β₂-adrenoreceptor agonists (e.g., albuterol [salbutamol], formoterol, pirbuterol, salmeterol and terbutaline); phosphodiesterase inhibitors, including PDE4 inhibitors (e.g., apremilast, cilomilast, ibudilast, piclamilast, roflumilast, crisaborole, diazepam, luteolin, mesembrenone, rolipram, AN2728 and E6005) and dual PDE3/4 inhibitors (e.g., tipelukast); specialized pro-resolving mediators (SPMs), including metabolites of polyunsaturated fatty acids (PUFAs) such as lipoxins (e.g., LXA4, 15-epi-LXA4, LXB4 and 15-epi-LXB4), resolvins (e.g., resolvins derived from 5Z,8Z,11Z,14Z,17Z-eicosapentaenoic acid [EPA], resolvins derived from 4Z,7Z,10Z,13Z,16Z,19Z-docosahexaenoic acid [DHA], and resolvins derived from 7Z,10Z,13Z,16Z,19Z-docosahexaenoic acid [n-3 DPA]), protectins/neuroprotectins (e.g., DHA-derived protectins/neuroprotectins and n-3 DPA-derived protectins/neuroprotectins), maresins (e.g., DHA-derived maresins and n-3 DPA-derived maresins), n-3 DPA metabolites, n-6 DPA (4Z,7Z,10Z,13Z,16Z-docosapentaenoic acid) metabolites, oxo-DHA metabolites, oxo-DPA metabolites, docosahexaenoyl ethanolamide metabolites, cyclopentenone prostaglandins (e.g., Δ12-PGJ₂ and 15-deoxy-Δ12,14-PGJ₂), and cyclopentenone isoprostanes (e.g., 5,6-epoxyisoprostane A₂ and 5,6-epoxyisoprostane E₂);

other kinds of anti-inflammatory agents, including pirfenidone, nintedanib, vitamin A, omega-3 fatty acids and esters thereof (e.g., docosahexaenoic acid [DHA], docosapentaenoic acid [DPA], eicosapentaenoic acid [EPA], α-linolenic acid [ALA], fish oils [which contain, e.g., DHA and EPA], and esters [e.g., glyceryl and ethyl esters] thereof), prostacyclin and analogs thereof (e.g., ataprost, beraprost [e.g., esuberaprost], 5,6,7-trinor-4,8-inter-m-phenylene-9-fluoro-PGI₂, carbacyclin, isocarbacyclin, clinprost [isocarbacyclin methyl ester], ciprostene, eptaloprost, cicaprost [metabolite of eptaloprost], iloprost, pimilprost, SM-10906 [des-methyl pimilprost], naxaprostene, taprostene, treprostinil, CS-570, OP-2507 and TY-11223), apoA-I mimetics (e.g., 4F), apoE mimetics (e.g., AEM-28 and AEM-28-14), and antioxidants (e.g., sulfur-containing antioxidants); and analogs, derivatives, fragments and salts thereof.

Non-steroidal anti-inflammatory drugs (NSAIDs) include without limitation: acetic acid derivatives, such as aceclofenac, bromfenac, diclofenac, etodolac, indomethacin, ketorolac, nabumetone, sulindac, sulindac sulfide, sulindac sulfone and tolmetin; anthranilic acid derivatives (fenamates), such as flufenamic acid, meclofenamic acid, mefenamic acid and tolfenamic acid; enolic acid derivatives (oxicams), such as droxicam, isoxicam, lornoxicam, meloxicam, piroxicam and tenoxicam; propionic acid derivatives, such as fenoprofen, flurbiprofen, ibuprofen, dexibuprofen, ketoprofen, dexketoprofen, loxoprofen, naproxen and oxaprozin; salicylates, such as diflunisal, salicylic acid, acetylsalicylic acid (aspirin), choline magnesium trisalicylate, salsalate and mesalazine; COX-2-selective inhibitors, such as apricoxib, celecoxib, etoricoxib, firocoxib, fluorocoxibs (e.g., fluorocoxibs A-C), lumiracoxib, mavacoxib, parecoxib, rofecoxib, tilmacoxib (JTE-522), valdecoxib, 4-O-methylhonokiol, niflumic acid, DuP-697, CG100649, GW406381, NS-398, SC-236, SC-58125, benzothieno[3,2-d]pyrimidin-4-one sulfonamide thio-derivatives, and COX-2 inhibitors derived from Tribulus terrestris; other kinds of NSAIDs, such as monoterpenoids (e.g., eucalyptol and phenols [e.g., carvacrol]), anilinopyridinecarboxylic acids (e.g., clonixin), sulfonanilides (e.g., nimesulide), and dual inhibitors of lipooxygenase (e.g., 5-LOX) and cyclooxygenase (e.g., COX-2) {e.g., chebulagic acid, licofelone, 2-(3,4,5-trimethoxyphenyl)-4-(N-methylindol-3-yl)thiophene, and di-tert-butylphenol-based compounds (e.g., DTPBHZ, DTPINH, DTPNHZ and DTPSAL)}; and analogs, derivatives and salts thereof.

The glucocorticoid class of corticosteroids has anti-inflammatory and immunosuppressive properties. Glucocorticoids include without limitation hydrocortisone types (e.g., cortisone and derivatives thereof [e.g., cortisone acetate], hydrocortisone and derivatives thereof [e.g., hydrocortisone acetate, hydrocortisone-17-aceponate, hydrocortisone-17-buteprate, hydrocortisone-17-butyrate and hydrocortisone-17-valerate], prednisolone, methylprednisolone and derivatives thereof [e.g., methylprednisolone aceponate], prednisone, and tixocortol and derivatives thereof [e.g., tixocortol pivalate]), betamethasone types (e.g., betamethasone and derivatives thereof [e.g., betamethasone dipropionate, betamethasone sodium phosphate and betamethasone valerate], dexamethasone and derivatives thereof [e.g., dexamethasone sodium phosphate], and fluocortolone and derivatives thereof [e.g., fluocortolone caproate and fluocortolone pivalate]), halogenated steroids (e.g., alclometasone and derivatives thereof [e.g., alclometasone dipropionate], beclometasone and derivatives thereof [e.g., beclometasone dipropionate], clobetasol and derivatives thereof [e.g., clobetasol-17-propionate], clobetasone and derivatives thereof [e.g., clobetasone-17-butyrate], desoximetasone and derivatives thereof [e.g., desoximetasone acetate], diflorasone and derivatives thereof [e.g., diflorasone diacetate], diflucortolone and derivatives thereof [e.g., diflucortolone valerate], fluprednidene and derivatives thereof [e.g., fluprednidene acetate], fluticasone and derivatives thereof [e.g., fluticasone propionate], halobetasol [ulobetasol] and derivatives thereof [e.g., halobetasol proprionate], halometasone and derivatives thereof [e.g., halometasone acetate], and mometasone and derivatives thereof [e.g., mometasone furoate]), acetonides and related substances (e.g., amcinonide, budesonide, ciclesonide, desonide, fluocinonide, fluocinolone acetonide, flurandrenolide [flurandrenolone or fludroxycortide], halcinonide, triamcinolone acetonide and triamcinolone alcohol), carbonates (e.g., prednicarbate), and analogs, derivatives and salts thereof. In certain embodiments, NRH, NARH or a reduced derivative thereof is used in vivo or ex vivo in combination with dexamethasone to treat a disorder or condition associated with a SARS-CoV-2 infection, such as COVID-19, SIRS or sepsis, or a complication thereof.

In further embodiments, NRH, NARH or a reduced derivative thereof (e.g., that of Formula I) is used in vivo or ex vivo in combination with an antioxidant to treat any disorder or condition disclosed herein. In some embodiments, the disorder or condition is associated with oxidative stress, damage or injury. In certain embodiments, the antioxidant is or comprises a vitamin or an analog thereof (e.g., vitamin E or an analog thereof such as α-tocopherol or trolox), a sulfur-containing antioxidant (e.g., glutathione, N-acetyl-L-cysteine or bucillamine), an ROS or radical scavenger (e.g., melatonin or glutathione), a mitochondrial antioxidant/“vitamin” (e.g., ubiquinone-10 [CoQ₁₀] or ubiquinol-10) or an analog thereof, or a mitochondria-targeted antioxidant (e.g., SkQ1, SkQR1, SkQT, SkQT1 or SkQTK1), or any combination thereof. In other embodiments, the antioxidant is or comprises a vitamin or an analog thereof (e.g., vitamin E or an analog thereof such as α-tocopherol or trolox), glutathione or a derivative thereof or an antioxidant that increases glutathione level (e.g., N-acetyl-L-cysteine optionally in combination with glycine), or a mitochondria-targeted antioxidant (e.g., SkQ1, MitoE, MitoQ or Mito-TEMPO), or any combination or all thereof.

Antioxidants include without limitation: vitamins and analogs thereof, including vitamin A, vitamin B₃ (e.g., niacin [nicotinic acid] and nicotinamide), vitamin C (ascorbic acid), vitamin E (including tocopherols [e.g., α-tocopherol] and tocotriernols), and vitamin E analogs (e.g., trolox [water-soluble]); carotenoids, including carotenes (e.g., β-carotene), xanthophylls (e.g., lutein, zeaxanthin and meso-zeaxanthin), and carotenoids in saffron (e.g., crocin and crocetin); sulfur-containing antioxidants, including glutathione (GSH), N-acetyl-L-cysteine (NAC), bucillamine, S-nitroso-N-acetyl-L-cysteine (SNAC), S-allyl-L-cysteine (SAC), S-adenosyl-L-methionine (SAM), α-lipoic acid and taurine; scavengers of ROS and radicals, including carnosine, N-acetylcarnosine, curcuminoids (e.g., curcumin, demethoxycurcumin and tetrahydrocurcumin), cysteamine, ebselen, glutathione, hydroxycinnamic acids and derivatives (e.g., esters and amides) thereof (e.g., caffeic acid, rosmarinic acid and tranilast), melatonin and metabolites thereof, nitrones (e.g., disufenton sodium [NXY-059]), nitroxides (e.g., XJB-5-131), polyphenols (e.g., flavonoids [e.g., apigenin, genistein, luteolin, naringenin and quercetin]), superoxide dismutase mimetics (infra), tirilazad, vitamin C, vitamin E and analogs thereof (e.g., α-tocopherol and trolox), and xanthine derivatives (e.g., pentoxifylline); mitochondrial antioxidants/“vitamins”, including ubiquinone (coenzyme Q, such as CoQ₁₀), ubiquinol (a reduced and more bioavailable form of ubiquinone, such as ubiquinol-10), ubiquinone/ubiquinol analogs (e.g., idebenone and mitoquinone) and derivatives; mitochondria-targeted antioxidants, including DMQ, DMMQ, MitoE, MitoQ, Mito-TEMPO, MitoVitE, and the SkQ class of compounds (e.g., SkQ1, SkQ2, SkQ3, SkQB, SkQR1, SkQT, SkQT1, SkQT1(m), SkQT1(p), SkQTK1, SkQTR1, SkQBerb and SkQPalm); inhibitors of enzymes that produce ROS, including NADPH oxidase (NOX) inhibitors (e.g., apocynin, decursin and decursinol angelate [both inhibit NOX-1, -2 and -4 activity and expression], diphenylene iodonium, and GKT-831 [formerly GKT-137831, a dual NOX1/4 inhibitor]), NADH:ubiquinone oxidoreductase (complex I) inhibitors (e.g., metformin and rotenone), xanthine oxidase inhibitors (e.g., allopurinol, oxypurinol, tisopurine, febuxostat, topiroxostat, myo-inositol, phytic acid, and flavonoids [e.g., kaempferol, myricetin and quercetin]), and myeloperoxidase inhibitors (e.g., azide, 4-aminobenzoic acid hydrazide and PF-06667272, and apoE mimetics such as AEM-28 and AEM-28-14); substances that mimic or increase the activity or production of antioxidant enzymes, including superoxide dismutase (SOD) {e.g., SOD mimetics such as manganese (III)- and zinc (III)-porphyrin complexes (e.g., MnTBAP, MnTMPyP and ZnTBAP), manganese (II) penta-azamacrocyclic complexes (e.g., M40401 and M40403), manganese (III)-salen complexes (e.g., those disclosed in U.S. Pat. No. 7,122,537) and OT-551 (a cyclopropyl ester prodrug of tempol hydroxylamine), and resveratrol and apoA-I mimetics such as 4F (both increase expression)}, catalase (e.g., catalase mimetics such as manganese (III)-salen complexes [e.g., those disclosed in U.S. Pat. No. 7,122,537], and zinc [increases activity]), glutathione peroxidase (GPx) (e.g., apomorphine and zinc [both increase activity], and beta-catenin, etoposide and resveratrol [all three increase expression]), glutathione reductase (e.g., 4-tert-butylcatechol and redox cofactors such as flavin adenine dinucleotide [FAD] and NADPH [all three enhance activity]), glutathione S-transferase (GST) (e.g., phenylalkyl isothiocyanate-cysteine conjugates {e.g., S-[N-benzyl(thiocarbamoyl)]-L-cysteine}, phenobarbital, rosemary extract and carnosol [all enhance activity]), thioredoxin (Trx) (e.g., geranylgeranylacetone, prostaglandin E₁ and sulforaphane [all increase expression]), NADPH-quinone oxidoreductase 1 (NQO1) {e.g., flavones [e.g., β-naphthoflavone (5,6-benzoflavone)] and triterpenoids [e.g., oleanolic acid analogs such as TP-151 (CDDO), TP-155 (CDDO methyl ester), TP-190, TP-218, TP-222, TP-223 (CDDO carboxamide), TP-224 (CDDO monomethylamide), TP-225, TP-226 (CDDO dimethylamide), TP-230, TP-235 (CDDO imidazolide), TP-241, CDDO monoethylamide, CDDO mono(trifluoroethyl)amide, and (+)-TBE-B], all of which increase expression by activating Nrf2}, heme oxygenase 1 (HO-1) {e.g., curcuminoids (e.g., curcumin), triterpenoids (e.g., oleanolic acid analogs such as TP-225), and apoA-I mimetics (e.g., 4F), all of which increase expression}, and paraoxonase 1 (PON-1) (e.g., apoE mimetics [e.g., AEM-28 and AEM-28-14] and apoA-I mimetics [e.g., 4F], both types increasing activity); activators of transcription factors that upregulate expression of antioxidant enzymes, including activators of nuclear factor (erythroid-derived 2)-like 2 (NFE2L2 or Nrf2) {e.g., bardoxolone methyl, OT-551, fumarates (e.g., dimethyl and monomethyl fumarate), dithiolethiones (e.g., oltipraz), flavones (e.g., β-naphthoflavone), isoflavones (e.g., genistein), sulforaphane, trichostatin A (also upregulates glutathione synthesis), triterpenoids (e.g., oleanolic acid analogs [e.g., TP-225]), and melatonin (increases Nrf2 expression)}; other kinds of antioxidants, including anthocyanins, benzenediol abietane diterpenes (e.g., carnosic acid), cyclopentenone prostaglandins (such as 15d-PGJ₂, which also upregulate glutathione synthesis), flavonoids {e.g., flavonoids in Ginkgo biloba (e.g., myricetin and quercetin [increases levels of GSH, SOD, catalase, GPx and GST]), prenylflavonoids (e.g., isoxanthohumol), flavones (e.g., apigenin), isoflavones (e.g., genistein), flavanones (e.g., naringenin) and flavanols (e.g., catechin and epigallocatechin-3-gallate)}, omega-3 fatty acids and esters thereof (supra), phenylethanoids (e.g., tyrosol and hydroxytyrosol), retinoids (e.g., all-trans retinol [vitamin A]), stilbenoids (e.g., resveratrol), uric acid, apoA-I mimetics (e.g., 4F), apoE mimetics (e.g., AEM-28 and AEM-28-14), and minerals (e.g., selenium and zinc [e.g., zinc monocysteine]); and analogs, derivatives and salts thereof.

In additional embodiments, NRH, NARH or a reduced derivative thereof (e.g., that of Formula I) is used in vivo or ex vivo in combination with an antifibrotic agent to treat a fibrotic disorder. In some embodiments, the antifibrotic agent is or comprises an anti-inflammatory agent (e.g., an inhibitor of TNF-α or its receptor or its production) or/and an antioxidant (e.g., vitamin E or an analog thereof such as α-tocopherol or trolox, a sulfur-containing antioxidant such as glutathione or taurine, or an ROS or radical scavenger such as melatonin, or any combination or all thereof). In certain embodiments, the antifibrotic agent is or comprises pirfenidone (which among its various antifibrotic and anti-inflammatory properties described herein also reduces fibroblast proliferation) or/and nintedanib (which blocks signaling of fibroblast growth factor receptors [FGFRs], platelet-derived growth factor receptors [PDGFRs] and vascular endothelial growth factor receptors [VEGFRs] involved in fibroblast proliferation, migration and transformation).

In some embodiments, the antifibrotic agent is or comprises an agent that has anti-hyperglycemic or/and insulin-sensitizing activity for treatment of a fibrotic disorder in which hyperglycemia, diabetes or insulin resistance contributes to development of fibrosis. Examples of such a disorder include diabetic nephropathy, which is characterized by renal fibrosis, and NASH and cirrhosis, both of which are characterized by hepatic fibrosis. Use of an anti-hyperglycemic or/and insulin-sensitizing agent can curtail or prevent, e.g., renal inflammation and fibrosis or hepatic inflammation and fibrosis. In certain embodiments, the antifibrotic agent is or comprises a PPAR-γ agonist (e.g., a thiazolidinedione [supra], such as pioglitazone or rosiglitazone). PPARγ-activating thiazolidinediones have both anti-hyperglycemic and insulin-sensitizing properties.

Antifibrotic agents include without limitation: inhibitors of collagen accumulation, including protein kinase C (PKC) inhibitors (e.g., BIM-1 BIM-2, BIM-3, BIM-8, chelerythrine, cicletanine, gossypol, miyabenol C, mnyricitrin, ruboxistaurin and verbascoside, which inhibit collagen production), 5-lipoxygenase inhibitors (e.g., tipelukast, which reduces collagen I, LOXL2 and TIMP-1 production), colchicine and its metabolite colchiceine (both inhibit collagen synthesis and deposition), dilinoleoyl-phosphatidylcholine (inhibits collagen production induced by transforming growth factor-beta1 [TGF-β1]), luteolin (reduces fibrosis in part by increasing expression of matrix metalloproteinase 9 [MMP-9] and metallothionein, which degrade the extracellular matrix [ECM]), malotilate (reduces procollagen I α2 [Col1a2] expression), melatonin (inhibits expression of procollagens I and III), S-nitroso-N-acetyl-L-cysteine (reduces collagen I amount in part by activating MMP-13 and suppressing tissue inhibitor of metalloproteinases 2 [TIMP-2]), oxymatrine {reduces procollagen I α1 (Col1a1) (and α-smooth muscle actin [α-SMA]) expression}, pioglitazone (reduces collagen I [and α-SMA] production), pirfenidone (reduces production of procollagens I and II and inhibits TGF-β-stimulated collagen production), quercetin (reduces Collal and procollagen III α1 [Col3a1] expression), resveratrol (reduces collagen I [and α-SMA] production), RGD mimetics and analogs (infra, reduce collagen I accumulation in part by increasing secretion of collagenases), safironil (reduces collagen I [and α-SMA] production), statins (e.g., atorvastatin, lovastatin and simvastatin [all three reduce collagen production]), tranilast (inhibits procollagen expression and fibroblast proliferation), valproic acid (reduces collagen deposition), inhibitors of collagen cross-linking {e.g., D-penicillamine and lysyl oxidase-like 2 (LOXL2, which promotes collagen cross-linking) inhibitors (e.g., β-aminopropionitrile and anti-LOXL2 antibodies [e.g., simtuzumab and AB-0023])}, procollagen-proline dioxygenase (or prolyl 4-hydroxylase, which forms more stable hydroxylated collagen) inhibitors (e.g., malotilate, HOE-077, S-0885 and S-4682), and procollagen glucosyltransferase (or galactosylhydroxylysine glucosyltransferase, which is important for collagen fibril formation) inhibitors (e.g., malotilate); inhibitors of pro-fibrotic growth factors (e.g., transforming growth factor-beta [including TGF-β1], connective tissue growth factor [CTGF] and platelet-derived growth factor [including PDGF-B, PDGF-C and PDGF-D]) or their production, activation or signaling, including TGF-β inhibitors {e.g., anti-TGF-3 antibodies (e.g., fresolimumab [GC1008] and CAT-192) and soluble TGF-β receptors (e.g., sTGFPR1, sTGFPR2 and sTGFPR3)}, TGFβR antagonists {e.g., TGFβR1 (ALK5) antagonists (e.g., galunisertib [LY-2157299], EW-7197, GW-788388, LY-2109761, SB-431542, SB-525334, SKI-2162, SM-16, and inhibitory Smads [e.g., Smad6 and Smad7])}, anti-CTGF antibodies (e.g., FG-3019), PDGF inhibitors (e.g., squalamine, PP1, anti-PDGF aptamers [e.g., E10030], anti-PDGF antibodies [e.g., those targeting PDGF-B, PDGF-C and PDGF-D], and soluble PDGF receptors [e.g., sPDGFRα and sPDGFRβ]), PDGFR (e.g., PDGFRα or/and PDGFRβ) antagonists (e.g., anti-PDGFR antibodies [e.g., REGN2176-3]), bone morphogenic protein-7 (BMP-7) (directly antagonizes TGF-β1 signaling and Smad3 activation, and promotes mesenchymal-to-epithelial transition), decorin (inhibits TGF-β1 activity and collagen fiber formation), N-acetyl-L-cysteine (inhibits TGF-β expression and activation by monomerization of the biologically active TGF-β dimer), S-nitroso-N-acetyl-L-cysteine (suppresses TGF-β1), L-carnitine (reduces PDGF-B expression), epigallocatechin-3-gallate (suppresses activation of Smad2 and Smad3 [and Akt]), galectin-7 (binds to and inhibits phosphorylated Smad2 and Smad3), Leu-Ser-Lys-Leu (inhibits TGF-01 activation), α-lipoic acid (inhibits TGF-β signaling via inhibition of Smad3 and AP-1), luteolin (inhibits TGF-β and PDGF signaling), melatonin (inhibits TGF-β and CTGF expression and Smad3 activation), naringenin (suppresses Smad3 expression and activation), niacin (reduces TGF-β expression), pirfenidone (reduces TGF-β production), quercetin (reduces expression of TGF-β1, CTGF, PDGF-B and Smad3), resveratrol (suppresses TGF-β expression), simvastatin (reduces TGF-β1 [and α-SMA] expression), taurine (reduces TGF-β1 [and α-SMA] expression), tranilast (inhibits TGF-β1 expression), vitamin E and analogs thereof (e.g., α-tocopherol and trolox, both of which suppress TGF-β expression), and α_{V}β₆ integrin (which activates TGF-β1) inhibitors (e.g., anti-α_{V}β₆ antibodies such as STX-100); receptor tyrosine kinase (TK) inhibitors, including epidermal growth factor receptor (EGFR) TK inhibitors (e.g., afatinib, brigatinib, erlotinib, gefitinib, icotinib, lapatinib, osimertinib and isoflavones [e.g., genistein]), PDGFR TK inhibitors (e.g., crenolanib, imatinib and AG-1295), dual FGFR/VEGFR TK inhibitors (e.g., brivanib and brivanib alaninate), dual PDGFR/VEGFR TK inhibitors (e.g., axitinib, sorafenib, sunitinib, vatalanib and X-82), and triple FGFR/PDGFR/VEGFR TK inhibitors (e.g., nintedanib and pazopanib); anti-EGFR antibodies, such as cetuximab, matuzumab, nimotuzumab, panitumumab and zalutumumab; anti-inflammatory agents, including those listed above, such as anti-inflammatory cytokines (e.g., IL-10), inhibitors of pro-inflammatory cytokines or their receptors or their production (e.g., TNF-α [e.g., an anti-TNF-α antibody such as infliximab or an immunomodulator such as pentoxifylline], IL-1β, IL-2, IL-6 and MCP-1), colchicine, curcuminoids (e.g., curcumin), malotilate, nintedanib, pirfenidone and tranilast; antioxidants, including those listed above, such as vitamins and analogs thereof (e.g., vitamin E and analogs thereof such as α-tocopherol and trolox), sulfur-containing antioxidants (e.g., glutathione, NAC, SNAC, SAC [also suppresses α-SMA expression], SAM and taurine), ROS and radical scavengers (e.g., melatonin and glutathione), Nrf2 activators {e.g., fumarates (e.g., dimethyl and monomethyl fumarate), trichostatin A, and triterpenoids (e.g., oleanolic acid analogs [supra, such as TP-225])}, and omega-3 fatty acids and esters thereof (e.g., Lovaza fish oil); antagonists of the renin-angiotensin-aldosterone system (RAAS), including renin inhibitors (e.g., aliskiren [reduces hepatic steatosis, oxidative stress, inflammation and fibrosis]), angiotensin-converting enzyme (ACE) inhibitors (e.g., captopril [inhibits fibroblast proliferation and reduces fibrotic lung response] and perindopril [inhibits liver fibrosis]), and angiotensin II receptor type 1 (AT₁) antagonists (e.g., candesartan [inhibits liver fibrosis], irbesartan and losartan) (activation of AT₁ by angiotensin II activates phospholipase C [PLC], leading to increased cytosolic Ca²⁺ concentration and hence PKC stimulation, also activates tyrosine kinases and promotes ECM formation); inhibitors of the accumulation or effects of advanced glycation end-products (AGEs, which inter alia increase arterial stiffness and stimulate mesangial matrix expansion), including inhibitors of AGE formation (e.g., aminoguanidine, aspirin, benfotiamine, carnosine, α-lipoic acid, metformin, pentoxifylline, pimagedine, pioglitazone, pyridoxamine, taurine and vitamin C), cleavers of AGE crosslinks (e.g., aminoguanidine, N-phenacylthiazolium bromide, rosmarinic acid, alagebrium [ALT-711], ALT-462, ALT-486 and ALT-946), and inhibitors of AGE effects (e.g., natural phenols such as curcumin and resveratrol); other kinds of antifibrotic agents, including RGD mimetics and analogs (inhibit adhesion of fibroblasts and immune cells to ECM glycoproteins) (e.g., NS-11, SF-6,5 and GRGDS), galectin-3 (which is critical for liver fibrosis) inhibitors (e.g., GM-CT-01 and GR-MD-02), mannobufagenin inhibitors (e.g., resibufogenin, spironolactone and canrenone), trichostatin A (inhibits TGFβ1-induced epithelial-to-mesenchymal transition), and PPAR-γ agonists (e.g., thiazolidinediones [supra], saroglitazar and IVA-337); and analogs, derivatives, fragments and salts thereof.

In some embodiments, the additional therapeutic agent for treatment of SIRS is or comprises selenium, glutamine or an omega-3 fatty acid (e.g., eicosapentaenoic acid), or any combination or all thereof. In other embodiments, the additional therapeutic agent for treatment of sepsis or SIRS caused by a microbe is or comprises an antimicrobial (e.g., an antibiotic, antifungal or antiviral). In further embodiments, the additional therapeutic agent for treatment of SIRS- or sepsis-induced shock/septic shock, AKI with a pre-renal cause or type 1 HRS characterized by low blood pressure is or comprises a blood pressure-raising drug (a vasopressor, such as norepinephrine, epinephrine, dobutamine, or vasopressin or an analog thereof such as ornipressin or terlipressin) if low blood pressure persists depite administration of intravenous fluid. In still further embodiments, the additional therapeutic agent for treatment of AKI with a pre-renal cause or type 1 HRS characterized by low blood pressure is or comprises a drug that increases the strength of heart muscle contraction (an inotrope, such as dobutamine). In additional embodiments, the additional therapeutic agent for treatment of type 1 HRS characterized by low blood pressure is or comprises a drug that causes splanchnic vasoconstriction or inhibits splanchnic vasodilation (e.g., a vasopressin analog such as ornipressin or terlipressin, or a somatostatin analog such as octreotide), a vasopressor or systemic vasoconstrictor (e.g., an α₁-adrenergic agonist such as midodrine or noradrenaline), or a plasma volume expander (e.g., albumin), or any combination or all thereof. In other embodiments, the additional therapeutic agent for treatment of acetaminophen overdose or acetaminophen-induced liver injury or liver failure (e.g., ALF) is or comprises N-acetyl-L-cysteine.

In further embodiments, the additional therapeutic agent for treatment of cirrhosis or a complication thereof is or comprises an agonist of the vasopressin receptor 1A (V_{1A}R) or/and the vasopressin receptor 1B (V_{1B}R, also known as the vasopressin receptor 3). In certain embodiments, the agonist is a partial or selective V_{1A}R agonist. Agonists of V_{1A}R or/and V1BR include without limitation FE 204038 (partial V_{1A}R agonist), FE 204205 (partial V_{1A}R agonist), and vasopressin analogs {e.g., ornipressin (V_{1A}R agonist), terlipressin (triple V_{1A}R/V_{1B}R/V₂R agonist), and ID-[Leu⁴, Lys⁸]-vasopressin (selective V_{1B}R agonist)}. Complications of cirrhosis include without limitation cardiovascular dysfunction and failure, portal hypertension, hyperdynamic circulation, esophageal varices, variceal bleeding (including esophageal and gastric variceal bleeding), splenomegaly, liver dysfunction and failure, ascites, jaundice, hypogonadism, hepatic encephalopathy, renal dysfunction and failure, AKI, HRS, respiratory dysfunction and failure, and cachexia. Cirrhosis or complications thereof can be associated with another medical condition, such as an infection (e.g., an infection with a virus such as the hepatitis B or C virus), SIRS or sepsis. Beneficial effects of agonists of V_{1A}R or/and V_{1B}R, including partial and selective V_{1A}R agonists, in cirrhotic patients include without limitation reduction in intrahepatic resistance, portal pressure and ascites, increase in peripheral or systemic vascular resistance, and induction of mesenteric or splanchnic vasoconstriction. In some embodiments, the use of NRH, NARH or a reduced derivative thereof in combination with an agonist of V_{1A}R or/and V_{1B}R improves the safety (e.g., prevent or reduce potential side effects such as intestinal ischemia, SIRS, sepsis, and respiratory dysfunction and failure) or/and the efficacy (e.g., improve liver function or/and renal function) of the agonist.

In other embodiments, the additional therapeutic agent for treatment of cirrhosis or a complication thereof is or comprises an antagonist of the vasopressin receptor 2 (V₂R), which can be used alternative to or in addition to an agonist of V_{1A}R or/and V_{1B}R. V₂R antagonists include without limitation selective V₂R antagonists (e.g., lixivaptan, mozavaptan, satavaptan, tolvaptan and RWJ-351647) and dual V_{1A}R/V₂R antagonists (e.g., conivaptan). In certain embodiments, the complication of cirrhosis treated with a V₂R antagonist is or comprises hyponatremia (e.g., hypervolemic hyponatremia), water retention, ascites, portal hypertension or variceal bleeding, or any combination thereof.

For in vivo use, the optional additional therapeutic agent(s) independently can be administered in any suitable mode, including without limitation oral, parenteral (including intramuscular, intradermal, subcutaneous, intravascular, intravenous, intra-arterial, intraperitoneal, intracavitary, intramedullary, intrathecal and topical), and topical (including dermal/epicutaneous, transdermal, mucosal, transmucosal, intranasal [e.g., by nasal spray or drop], pulmonary [e.g., by oral or nasal inhalation], ocular [e.g., by eye drop], buccal, sublingual, rectal [e.g., by suppository] and vaginal [e.g., by suppository]). In certain embodiments, an additional therapeutic agent is administered orally. In other embodiments, an additional therapeutic agent is administered parenterally (e.g., intravenously, subcutaneously or intramuscularly).

For in vivo or ex vivo use, the optional additional therapeutic agent(s) independently can be administered or provided in any suitable frequency, including without limitation daily (one, two or more times per day), once every two or three days, thrice weekly, twice weekly or once weekly, or on a pro re nata (as-needed) basis, which can be determined by the treating physician. The dosing frequency can depend on, e.g., the mode of administration chosen. For in vivo or ex vivo use, the length of treatment with the optional additional therapeutic agent(s) can be determined by the treating physician and can independently be, e.g., at least about 1 day, 2 days, 3 days, 1 week, 2 weeks, 3 weeks, 4 weeks (1 month), 6 weeks, 2 months, 3 months, 6 months, 1 year, 2 years, 3 years, 4 years, 5 years or longer.

For in vivo or ex vivo use, the dose or therapeutically effective amount of, the frequency and route of administration/provision of, and the length of treatment with, an optional additional therapeutic agent can be based in part on recommendations for that therapeutic agent and can be determined by the treating physician.

For in vivo or ex vivo use, in some embodiments NRH, NARH or a reduced derivative thereof and an additional therapeutic agent are administered in separate pharmaceutical compositions. For in vivo or ex vivo use, in other embodiments NRH, NARH or a reduced derivative thereof and an additional therapeutic agent are administered in the same pharmaceutical composition, such as in a fixed-dose combination dosage form. In some embodiments, the fixed-dose combination dosage form is formulated for controlled-release, slow-release or sustained-release of NRH, NARH or a reduced derivative thereof or/and the additional therapeutic agent. In certain embodiments, the fixed-dose combination dosage form is formulated for oral administration, such as in the form of a tablet, capsule or pill. In other embodiments, the fixed-dose combination dosage form is formulated for parenteral administration, such as intravenously, subcutaneously or intramuscularly.

Reduced Derivatives of NRH and NARH

Reduced derivatives of NRH and NARH can function as prodrugs of NRH and NARH. In some embodiments, reduced derivatives of NRH and NARH have Formula I:

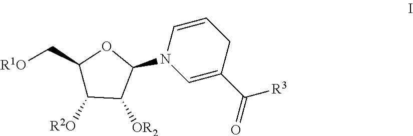

wherein:

R¹ is hydrogen,

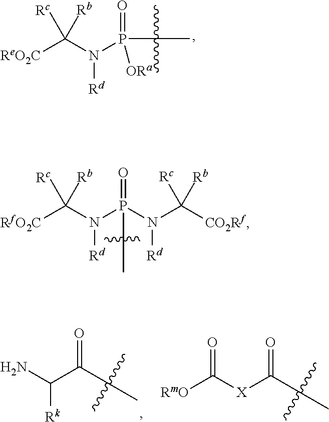

or —C(═O)R^{p}, wherein:

R^{a} is hydrogen, a counterion, linear or branched C₁-C₆ alkyl, C₃-C₆ cycloalkyl, phenyl, 1-naphthyl or 2-naphthyl, wherein the phenyl is optionally substituted with F, Cl, —CN, —NO₂, linear or branched C₁-C₄ alkyl, —CF₃, —O-(linear or branched C₁-C₄ alkyl) or —OCF₃;

R^{b} and R^{c} at each occurrence independently are hydrogen, linear or branched C₁-C₆ alkyl, —CH₂-phenyl, —CH₂-3-indole or —CH₂-4/5-imidazole, wherein the alkyl is optionally substituted with —OH, —OR^{j}, —SH, —SR^{j}, —NH₂, —NHR^{j}, —N(R^{j})₂, —NHC(═O)R^{j}, —NHC(═NH)NH₂, —C(═O)NH₂, —CO₂H or —C(═O)OR^{j}, and the phenyl is optionally substituted with —OH or —OR wherein R^{j} at each occurrence independently is linear or branched C₁-C₄ alkyl;

R^{d} at each occurrence independently is hydrogen or linear or branched C1-C₄ alkyl;

R^{e} and R^{f} at each occurrence independently are hydrogen, a counterion, linear or branched C₁-C₈ alkyl, C₃-C₆ cycloalkyl, —CH₂—(C₃-C₆ cycloalkyl), phenyl or —CH₂-phenyl, wherein the phenyl is optionally substituted with F, Cl, —CN, —NO₂, linear or branched C₁-C₄ alkyl, —CF₃, —O-(linear or branched C₁-C₄ alkyl) or —OCF₃;

R^{k} is hydrogen, linear or branched C₁-C₆ alkyl, —CH₂-phenyl, —CH₂-3-indole or —CH₂-4/5-imidazole, wherein the alkyl is optionally substituted with —OH, —OR^{j}, —SH, —SR^{j}, —NH₂, —NHR^{j}, —N(R^{j})₂, —NHC(═O)R^{j}, —NHC(═NH)NH₂, —C(═O)NH₂, —CO₂H or —C(═O)OR^{j}, and the phenyl is optionally substituted with —OH or —OR^{j}, wherein R^{j} at each occurrence independently is linear or branched C₁-C₄ alkyl;

R^{m} is hydrogen, a counterion, linear or branched C₁-C₆ alkyl, C₃-C₆ cycloalkyl, phenyl, —CH₂-phenyl or

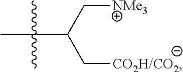

wherein the phenyl is optionally substituted with F, Cl, —CN, —NO₂, linear or branched C₁-C₄ alkyl, —CF₃, —O-(linear or branched C₁-C₄ alkyl) or —OCF₃;

X is cis or trans —HC═CH— or —(CH₂)ₙ— optionally substituted with —OH, —OR^{j} or —OC(═O)R^{j}, wherein R^{j} is linear or branched C₁-C₄ alkyl and n is 1, 2, 3, 4, 5 or 6; and

R^{p} is linear or branched C₁-C₆ alkyl, C₃-C₆ cycloalkyl, or phenyl optionally substituted with F, Cl, —CN, —NO₂, linear or branched C₁-C₄ alkyl, —CF₃, —O-(linear or branched C₁-C₄ alkyl) or —OCF₃;

R² at each occurrence independently is hydrogen,

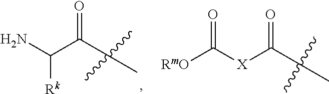

or —C(═O)R^{p}, wherein R^{k}, R^{m}, X and R^{p} are as defined above; and

R³ is —NH₂, —NHRⁿ, —N(Rⁿ)₂, —OH, —OR^{o} or

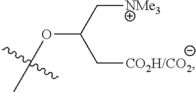

wherein:

Rⁿ at each occurrence independently is linear or branched C₁-C₆ alkyl or allyl, wherein the alkyl is optionally substituted with —OH or —O-(linear or branched C₁-C₃ alkyl), or both occurrences of Rⁿ and the nitrogen atom to which they are connected form a 3- to 6-membered heterocyclic ring; and

R^{o} is a counterion, linear or branched C₁-C₆ alkyl, C₃-C₆ cycloalkyl, phenyl or —CH₂-phenyl, wherein the phenyl is optionally substituted with F, Cl, —CN, —NO₂, linear or branched C₁-C₄ alkyl, —CF₃, —O-(linear or branched C₁-C₄ alkyl) or —OCF₃;

wherein R¹ is not hydrogen, both occurrences of R² are not hydrogen, and R³ is not —NH₂ or —OH or a salt thereof;

or a pharmaceutically acceptable salt, solvate, hydrate, clathrate, polymorph or stereoisomer thereof.

In certain embodiments, R¹ and both occurrences of R² all are not hydrogen except when R³ is

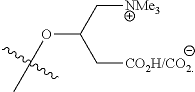

In other embodiments, R¹ is not hydrogen or —C(═O)-(linear or branched C₁-C₆ alkyl) and both occurrences of R² are not hydrogen or —C(═O)-(linear or branched C₁-C₆ alkyl) when R³ is —NH₂ or —OH or a salt thereof.

In yet other embodiments, when both occurrences of R² are acetyl:

R¹ is not hydrogen; or

R³ is not —NH₂ or —OH or a salt thereof; or

R¹ is not hydrogen and R³ is not —NH₂ or —OH or a salt thereof.

In further embodiments, when R¹ is

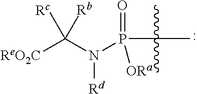

both occurrences of R² are not hydrogen; or

R³ is not —NH₂ or —OH or a salt thereof; or

both occurrences of R² are not hydrogen and R³ is not —NH₂ or —OH or a salt thereof.

In still further embodiments, when R¹ is

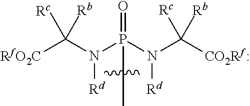

both occurrences of R² are not hydrogen; or

R³ is not —NH₂ or —OH or a salt thereof; or

both occurrences of R² are not hydrogen and R³ is not —NH₂ or —OH or a salt thereof.

In other embodiments, when R¹ is

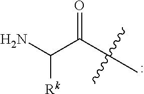

both occurrences of R² are not hydrogen; or

R³ is not —NH₂ or —OH or a salt thereof; or

both occurrences of R² are not hydrogen and R³ is not —NH₂ or —OH or a salt thereof.

In certain embodiments, a reduced derivative of NRH or NARH is not:

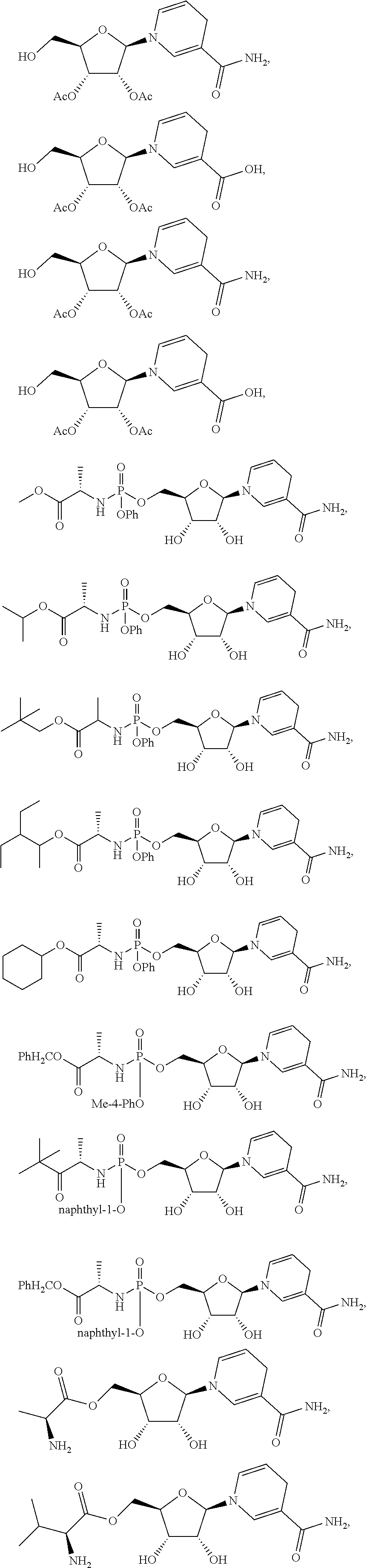

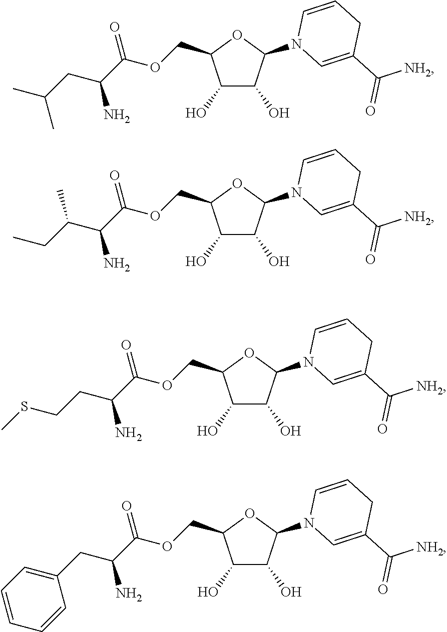 [SMILES: CC(C)C[C@H](N)C(=O)OC[C@H]1O[C@@H](N2C=CCC(C(N)=O)=C2)[C@H](O)[C@@H]1O ; CC[C@H](C)[C@H](N)C(=O)OC[C@H]1O[C@@H](N2C=CCC(C(N)=O)=C2)[C@H](O)[C@@H]1O ; CSCC[C@H](N)C(=O)OC[C@H]1O[C@@H](N2C=CCC(C(N)=O)=C2)[C@H](O)[C@@H]1O ; NC(=O)C1=CN([C@@H]2O[C@H](COC(=O)[C@@H](N)Cc3ccccc3)[C@@H](O)[C@H]2O)C=CC1]

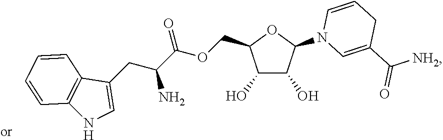 [SMILES: NC(=O)C1=CN([C@@H]2O[C@H](COC(=O)[C@@H](N)Cc3c[nH]c4ccccc34)[C@@H](O)[C@H]2O)C=CC1]

or a salt or stereoisomer thereof.

In some embodiments, R¹ is

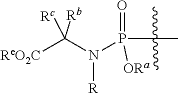

(phosphoramidate). In certain embodiments, R¹ is

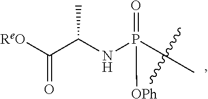

and R^{e} is linear or branched C₁-C₆ alkyl. In certain embodiments, R^{e} is methyl, ethyl or isopropyl.

In further embodiments, R¹ is

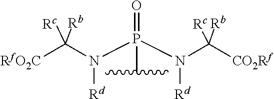

(phosphorodiamidate/bisphosphoramidate). In certain embodiments, R¹ is

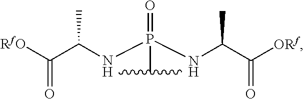

and both occurrences of R^{f} are linear or branched C₁-C₆ alkyl. In certain embodiments, both occurrences of R^{f} are methyl, ethyl or isopropyl.

In other embodiments, R¹ is

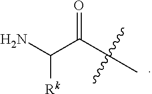

In certain embodiments, R¹ is

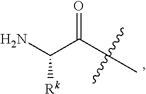

and R^{k} is hydrogen or linear or branched C₁-C₆ alkyl. In certain embodiments, R^{k} is hydrogen, methyl, ethyl or isopropyl. An amino acid group can facilitate penetration of an NRH/NARH derivative through membrane barriers via peptide transporters, such as peptide transporter 1 in the intestinal epithelium.

In additional embodiments, R¹, or/and R² at either occurrence or at both occurrences, is/are

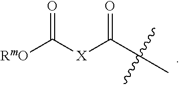

In some embodiments, X is trans —HC═CH—, —CH₂CH₂— or —CH(OH)CH₂—, and R^{m} is hydrogen, a counterion, linear or branched C₁-C₆ alkyl (e.g., methyl, ethyl or isopropyl) or

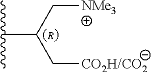

(an L-carnitine group). The —CH(OH)CH₂— portion can have the S-stereochemistry or a mixture (e.g., an approximately 1:1 ratio) of S/R-stereochemistry. In certain embodiments, R¹, or/and R² at either occurrence or at both occurrences, is/are selected from:

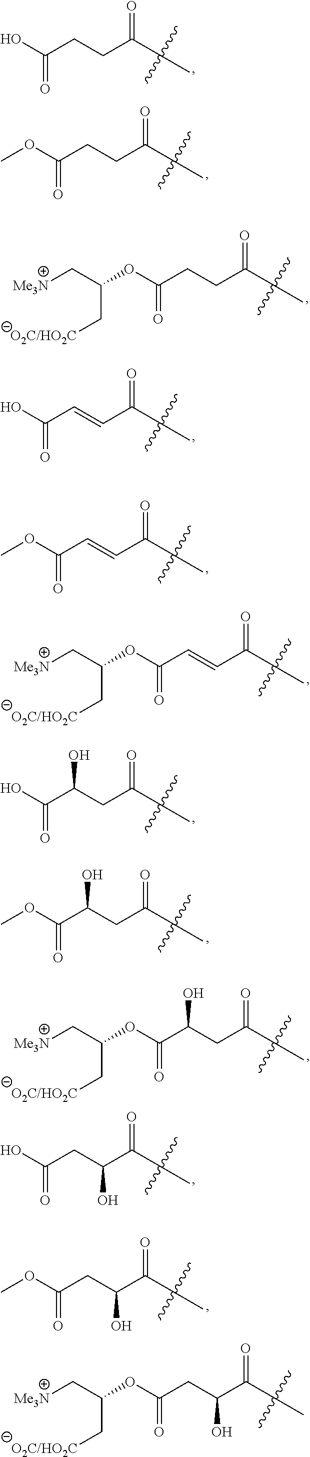

and salts thereof. A carnitine group can facilitate transport of an NRH/NARH derivative into the mitochondria.

In some embodiments, R² at each occurrence independently, or at both occurrences, is hydrogen, —C(═O)-(linear or branched C₁-C₆ alkyl),

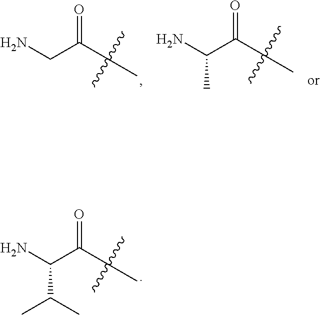

In certain embodiments, R² at each occurrence independently, or at both occurrences, is hydrogen, acetyl or propanoyl.

If a compound of Formula I comprises an amino acid group at R¹ or/and at either occurrence or both occurrences of R², including an amino acid group in a phosphoramidate moiety at R¹ or two amino acid groups in a phosphorodiamidate/bisphosphoramidate moiety at R¹, the amino acid group can independently be a natural amino acid or an unnatural amino acid. In some embodiments, an amino acid group is glycine, alanine, valine, leucine, isoleucine, methionine, proline, tryptophan, phenylalanine, tyrosine, serine, threonine, cysteine, asparagine, glutamine, aspartic acid, glutamic acid, lysine, arginine or histidine, or a derivative thereof. In other embodiments, an amino acid group is an unnatural or non-proteinogenic amino acid, such as ornithine, citrulline or homoarginine. In certain embodiments, an amino acid group is glycine, alanine or valine. An amino acid group can be the L-isomer or the D-isomer, or can be a D/L (e.g., racemic) mixture. In certain embodiments, an amino acid group is the L-isomer.

In some embodiments, R³ is —NH₂, —OH or a salt thereof, or

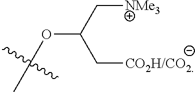

In certain embodiments, R³ is

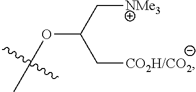

an L-carnitine moiety. The carnitine moiety can exist as a zwitterion or in a salt form where the quaternary ammonium ion has a counterion.

In some embodiments of compounds of Formula I.

R¹ is

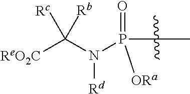

and both occurrences of R² are acetyl or propanoyl; or

R¹ is

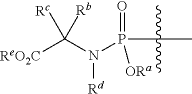

and R³ is —NH₂ or —OH or a salt thereof, or

R¹ is

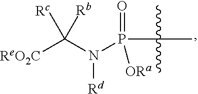

both occurrences of R² are acetyl or propanoyl, and R³ is —NH₂ or —OH or a salt thereof.

In certain embodiments, R¹ is

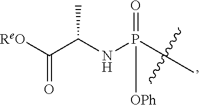

and R^{e} is linear or branched C₁-C₆ alkyl. In certain embodiments, R^{e} is methyl, ethyl or isopropyl.

In further embodiments of compounds of Formula I:

R¹ is

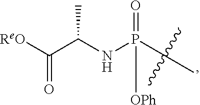

wherein R^{e} is linear or branched C₁-C₆ alkyl;

R² at both occurrences is —C(═O)-(linear or branched C₁-C₆ alkyl); and

R³ is —NH₂ or —OH or a salt thereof.

In certain embodiments, R^{e} of the R¹ moiety is methyl, ethyl or isopropyl, and both occurrences of R² are acetyl or propanoyl.

In other embodiments of compounds of Formula I:

R¹ is

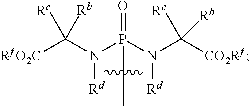

R² at each occurrence independently, or at both occurrences, is hydrogen, acetyl or propanoyl; and

R³ is —NH₂ or —OH or a salt thereof.

In some embodiments:

R^{b} and R^{c} at each occurrence independently are hydrogen or linear or branched C₁-C₆ alkyl, or each pair of R^{b} and R^{c} is hydrogen and linear or branched C₁-C₆ alkyl;

R^{d} at both occurrences is hydrogen; and

R^{f} at both occurrences is linear or branched C₁-C₆ alkyl.

In certain embodiments, R¹ is

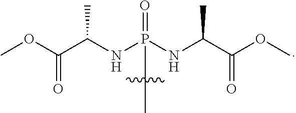

In further embodiments of compounds of Formula I:

R¹ is

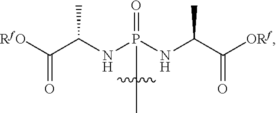

wherein both occurrences of R^{f} are linear or branched C₁-C₆ alkyl;

R² at each occurrence independently, or at both occurrences, is hydrogen or —C(═O)-(linear or branched C₁-C₆ alkyl); and

R³ is —NH₂ or —OH or a salt thereof.

In certain embodiments, both occurrences of R^{f} of the R¹ moiety are methyl, ethyl or isopropyl, and R² at each occurrence independently, or at both occurrences, is hydrogen, acetyl or propanoyl.

In additional embodiments of compounds of Formula I:

R¹ is

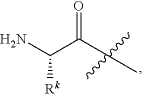

wherein R^{k} is hydrogen or linear or branched C₁-C₆ alkyl;

R² at each occurrence independently, or at both occurrences, is hydrogen or —C(═O)-(linear or branched C₁-C₆ alkyl); and

R³ is —NH₂ or —OH or a salt thereof.

In certain embodiments, R^{k} of the R¹ moiety is hydrogen, methyl, ethyl or isopropyl, and R² at each occurrence independently, or at both occurrences, is hydrogen, acetyl or propanoyl.

In other embodiments of compounds of Formula I:

R¹ is

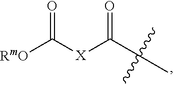

wherein:

X is cis or trans —HC═CH— or —(CH₂)ₙ— optionally substituted with —OH, —OR^{j} or —OC(═O)R^{j}, wherein R^{j} is linear or branched C₁-C₄ alkyl and n is 1, 2, 3, 4, 5 or 6; and

R^{m} is hydrogen, a counterion, linear or branched C₁-C₆ alkyl or

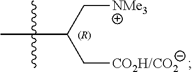

R² at each occurrence independently, or at both occurrences, is hydrogen or —C(═O)-(linear or branched C₁-C₆ alkyl); and

R³ is —NH₂ or —OH or a salt thereof.

In certain embodiments:

for the R¹ moiety, X is trans —HC═CH—, —CH₂CH₂— or —CH(OH)CH₂—, and R^{m} is hydrogen, a counterion, methyl, ethyl, isopropyl or

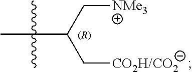

R² at each occurrence independently, or at both occurrences, is hydrogen, acetyl or propanoyl; and

R³ is —NH₂.

In further embodiments of compounds of Formula I:

R¹ is hydrogen or —C(═O)R^{p}, wherein R^{p} is linear or branched C₁-C₆ alkyl;

R² at each occurrence independently, or at both occurrences, is hydrogen or —C(═O)R^{p}, wherein R^{p} is linear or branched C₁-C₆ alkyl; and

R³ is —NH₂, —OH or a salt thereof, or

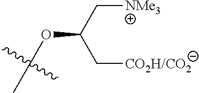

### (L-carnitine).

In certain embodiments, R¹ is hydrogen, acetyl or propanoyl, and R² at each occurrence independently, or at both occurrences, is hydrogen, acetyl or propanoyl.

In some embodiments, reduced derivatives of NRH and NARH of Formula I are selected from:

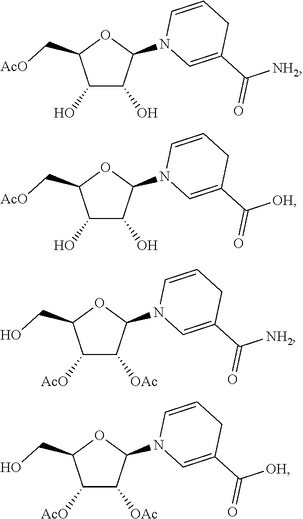

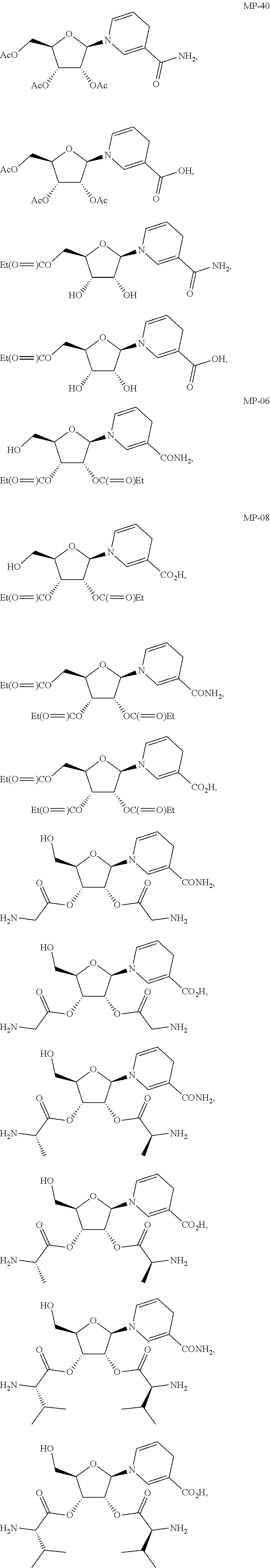

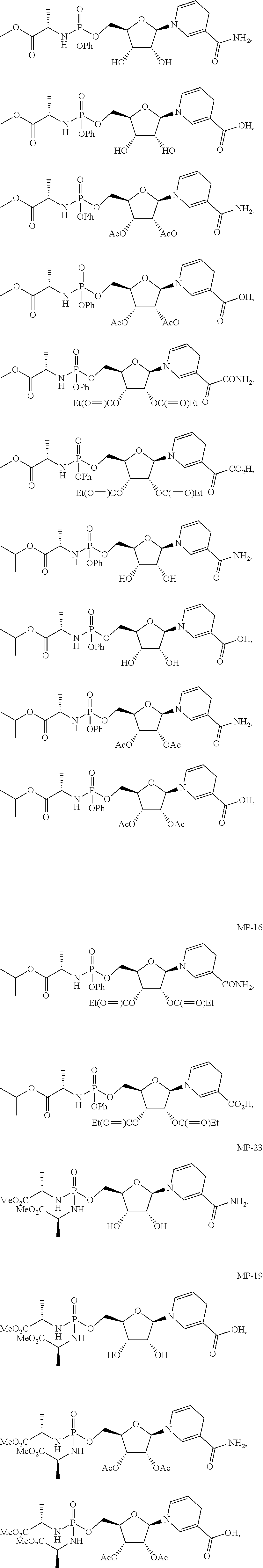

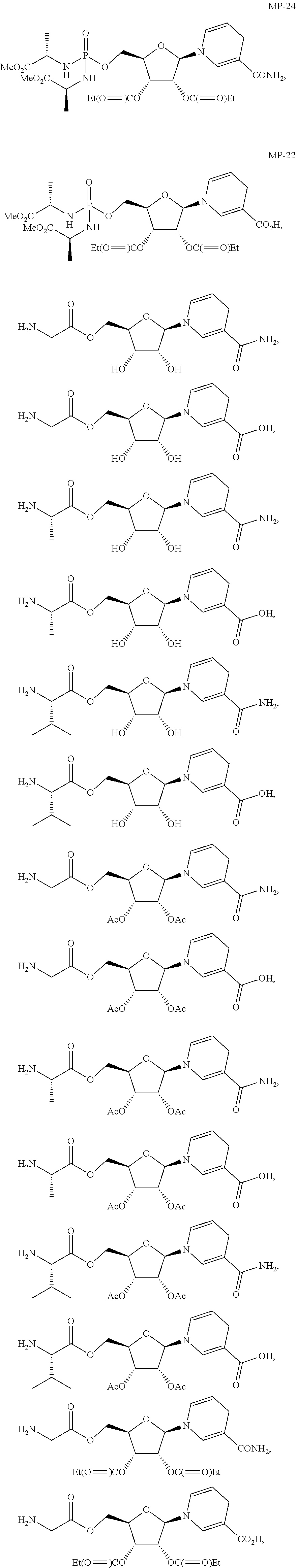

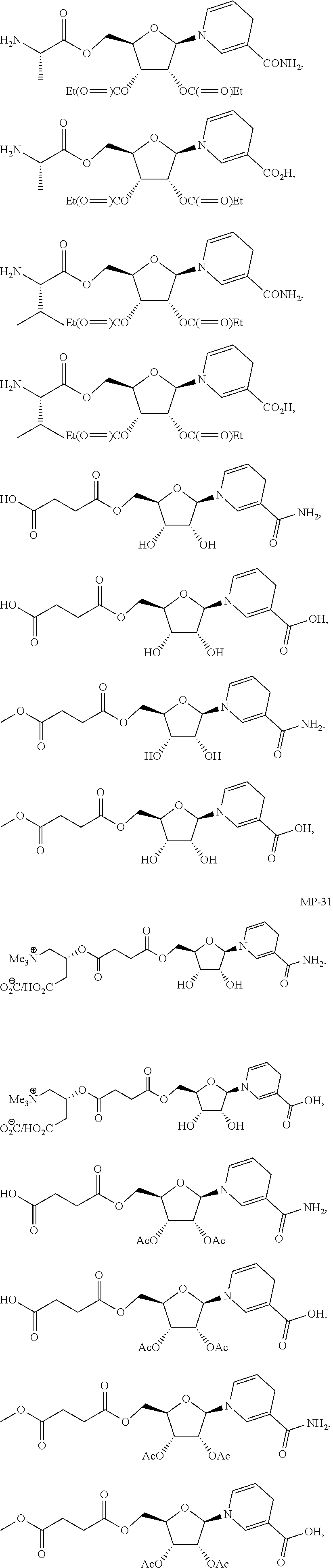

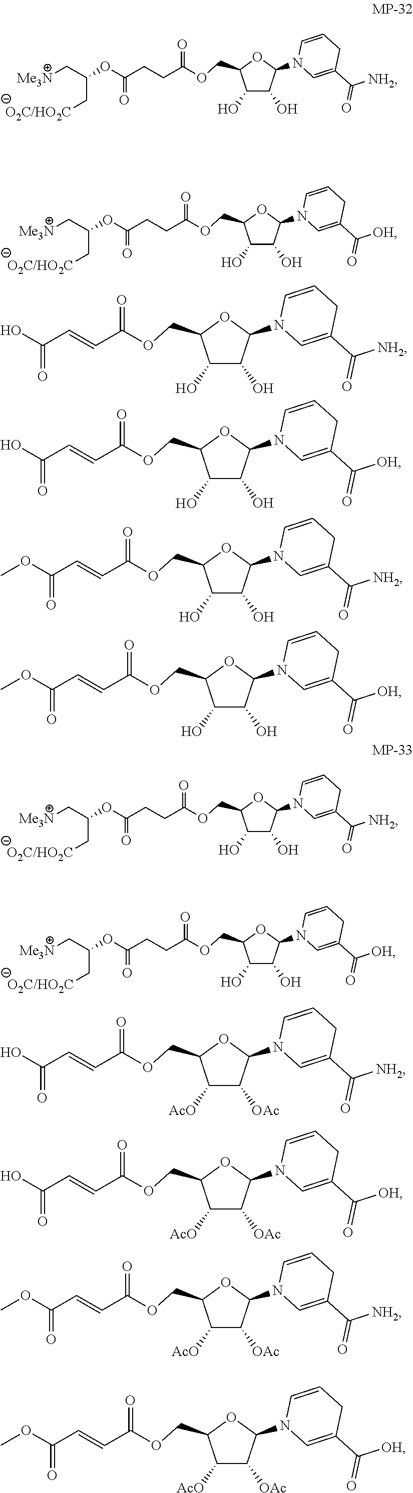

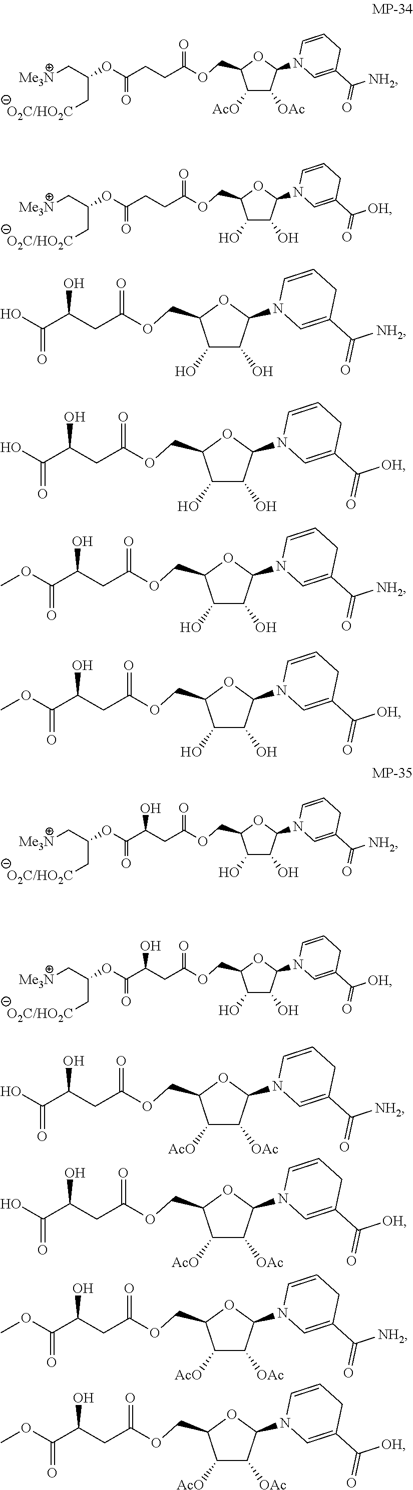

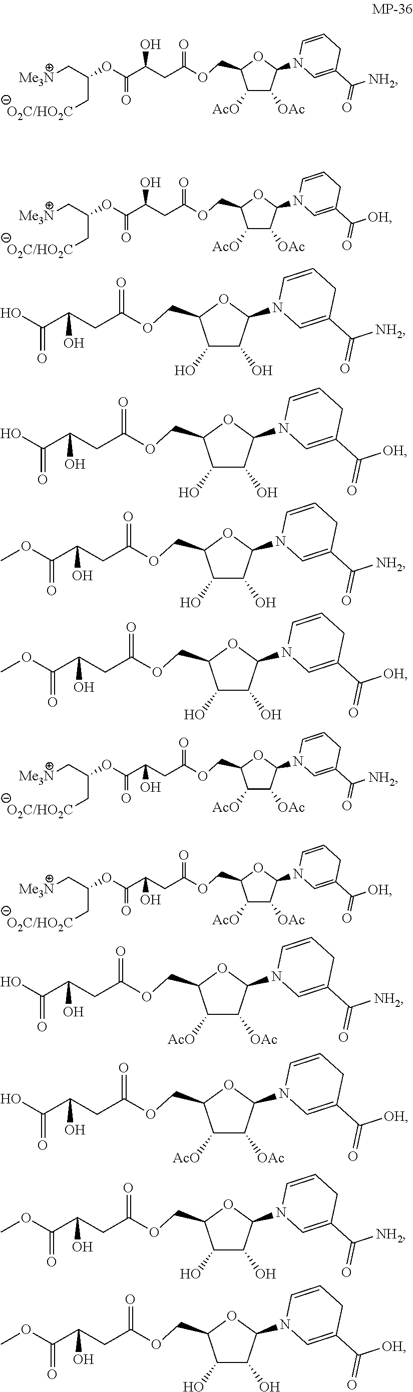

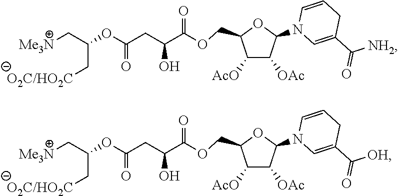

and pharmaceutically acceptable salts, solvates, hydrates, clathrates, polymorphs and stereoisomers thereof.

In other embodiments, reduced derivatives of NARH of Formula I are selected from:

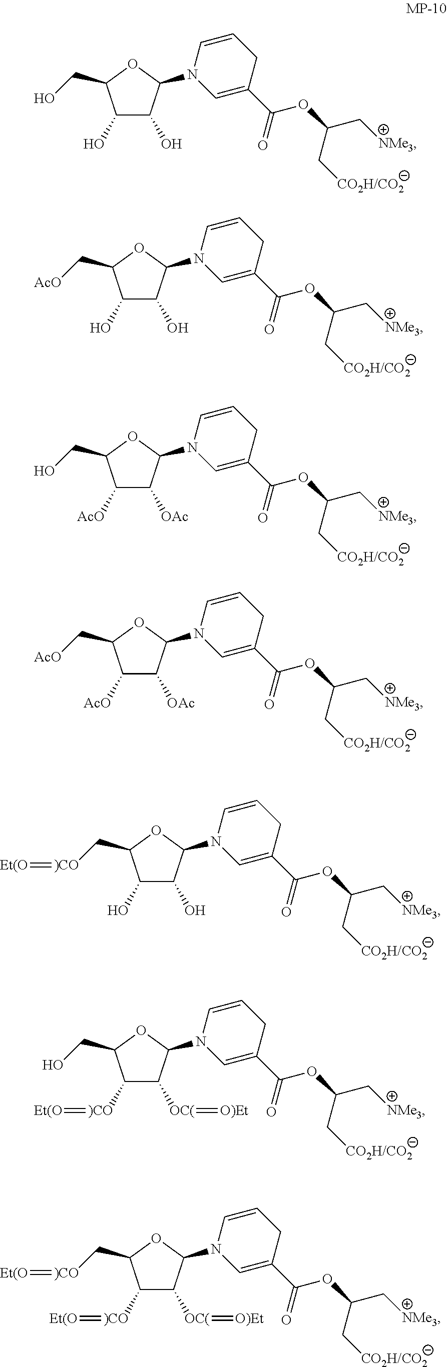

and pharmaceutically acceptable salts, solvates, hydrates, clathrates, polymorphs and stereoisomers thereof.

As modification of Formula I, reduced derivatives of NRH and NARH of Formula I can comprise a hydrophobic/lipophilic group at R¹ at either occurrence or both occurrences of R² or at R³, or any combination thereof. One or more hydrophobic groups can facilitate permeation of an NRH/NARH derivative through membrane barriers, including the cell membrane. In certain embodiments, a hydrophobic group contains 6-20, 8-20, 10-18 or 12-16 carbon atoms. In some embodiments, a hydrophobic group is a linear or branched, saturated (e.g., acyl or alkyl) group containing 6-20, 8-20, 10-18 or 12-16 carbon atoms, such as a linear saturated (e.g., acyl or alkyl) group containing 6, 8, 10, 12, 14, 16, 18 or 20 carbon atoms. In other embodiments, a hydrophobic group is a linear unsaturated (e.g., acyl or alkenyl) group containing 8-20 (e.g., 8, 10, 12, 14, 16, 18 or 20) carbon atoms and having 1, 2, 3 or 4 C═C double bonds, each of which can independently be cis or trans. In some embodiments of compounds of Formula I:

R^{a} can be linear or branched C₁-C₂₀ alkyl or alkenyl;

R^{b} or R^{c} can be linear or branched C₁-C₂₀ alkyl or alkenyl for a phosphoramidate moiety;

R^{b} or R^{c} at either occurrence or both occurrences can be linear or branched C₁-C₂₀ alkyl or alkenyl for a phosphorodiamidate/bisphosphoramidate moiety;

R^{e} can be linear or branched C₁-C₂₀ alkyl or alkenyl;

R^{f} at either occurrence or both occurrences can be linear or branched C₁-C₂₀ alkyl or alkenyl;

R^{k} at any occurrence can be linear or branched C₁-C₂₀ alkyl or alkenyl;

R^{m} at any occurrence can be linear or branched C₁-C₂₀ alkyl or alkenyl;

Rⁿ at any occurrence can be linear or branched C₁-C₂₀ alkyl or alkenyl;

R^{o} can be linear or branched C₁-C₂₀ alkyl or alkenyl;

R^{p} at any occurrence can be linear or branched C₁-C₂₀ alkyl or alkenyl; or

n for —(CH₂)ₙ— for X at any occurrence can be an integer from 1 to 20, and —(CH₂)ₙ— for X can have one or more C═C double bonds; or

any combination of the above.

The disclosure also encompasses isotopologues of NRH, NARH and reduced derivatives thereof (including those of Formula I). Isotopically enriched forms of NRH, NARH and reduced derivatives thereof include without limitation those enriched in the content of 2H (deuterium), ¹³C, ¹⁵N, ¹⁷O or ¹⁸O, or any combination thereof, at one or more, or all, positions of the corresponding atom(s).

Isomers of Compounds

The present disclosure encompasses all possible stereoisomers, including both enantiomers and all possible diastereomers in substantially pure form and mixtures of both enantiomers in any ratio (including a racemic mixture of enantiomers) and mixtures of two or more diastereomers in any ratio, of the compounds described herein, and not only the specific stereoisomers as indicated by drawn structure or nomenclature. In preferred embodiments, the disclosure relates to the specific stereoisomers indicated by drawn structure or nomenclature, including the beta-anomer of dihydronicotinamide D-riboside (NRH), dihydronicotinic acid D-riboside (NARH) and reduced derivatives thereof (including those of Formula I) The specific recitation of the phrase “or stereoisomers thereof” or the like with respect to a compound in certain instances of the disclosure shall not be interpreted as an intended omission of any of the other possible stereoisomers of the compound in other instances of the disclosure where the compound is mentioned without recitation of the phrase “or stereoisomers thereof” or the like, unless stated otherwise or the context clearly indicates otherwise.

In some embodiments, NRH, NARH and reduced derivatives thereof (including those of Formula I) are stereoisomerically pure. In some embodiments, at least about 90%, 95%, 97%, 98% or 99% of NRH, NARH and reduced derivatives thereof have the stereochemistry indicated by drawn structure or nomenclature, including the beta-D-riboside configuration. In similar embodiments, NRH, NARH and reduced derivatives thereof have the beta-D-riboside configuration and an enantiomeric excess of at least about 80%, 90% or 95%.

In other embodiments, NRH, NARH and reduced derivatives thereof (including those of Formula I) are mixtures of enantiomers or mixtures of two or more diastereomers. In certain embodiments, NRH, NARH and reduced derivatives thereof are racemic mixtures. In other embodiments, NRH, NARH and reduced derivatives thereof have the D-riboside configuration and a mixture of beta-/alpha-anomers. In certain embodiments, NRH, NARH and reduced derivatives thereof have the D-riboside configuration and an approximately 1:1 ratio of beta-/alpha-anomers.

Salt Forms of Compounds

NRH, NARH and reduced derivatives thereof (including those of Formula I) may exist as salts, such as if the glycosidic nitrogen atom is protonated. The disclosure encompasses all pharmaceutically acceptable salts of NRH, NARH and reduced derivatives thereof. Examples of counteranions of salts of NRH, NARH and reduced derivatives thereof (including those of Formula I), including if the glycosidic nitrogen atom is protonated and including the salt form of the carnitine moiety

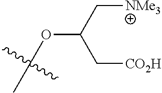

if the carnitine moiety is not in the zwitterionic form

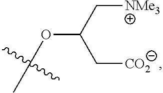

include without limitation internal salt, fluoride, chloride, bromide, iodide, nitrate, sulfate, sulfite, phosphate, bicarbonate, carbonate, thiocyanate, formate, acetate, trifluoroacetate, glycolate, lactate, gluconate, ascorbate, benzoate, oxalate, malonate, succinate, citrate, methanesulfonate (mesylate), ethanesulfonate, propanesulfonate, benzenesulfonate (bezylate), p-toluenesulfonate (tosylate) and trifluoromethanesulfonate (triflate). In certain embodiments, the counteranion of salts of NRH, NARH and reduced derivatives thereof, such as if the glycosidic nitrogen atom is protonated or/and if the carnitine moiety is in the salt form rather than the zwitterionic form, is chloride, formate, acetate, trifluoroacetate or triflate.

NARH has an acidic group, and reduced derivatives of NRH and NARH (including those of Formula I) may have acidic group(s), such as carboxylic acid group(s) or/and a phosphoric acid group. Such compounds may form salt(s) with the acidic group(s). The countercation(s) can be, e.g., Li⁺, Na⁺, K⁺, Ca⁺², Mg⁺², ammonium, a protonated organic amine (e.g., diethanolamine) or a quaternary ammonium compound (e.g., choline).

Pharmaceutical Compositions

The disclosure provides pharmaceutical compositions comprising NRH, NARH or a reduced derivative thereof (e.g., that of Formula I), or a pharmaceutically acceptable salt, solvate, hydrate, clathrate, polymorph or stereoisomer thereof, and one or more pharmaceutically acceptable excipients or carriers. The compositions can optionally contain an additional therapeutic agent. A pharmaceutical composition generally contains a therapeutically effective amount of the active ingredient (for treating, e.g., an immune-related disorder such as SIRS or sepsis, a kidney disorder such as AKI or HRS, a liver disorder such as ALF or HRS, a hemolytic disorder such as hemolysis or hemolytic anemia, or a disorder or condition associated with oxidative stress, damage or injury such as methemoglobinemia or anemia), but can contain an appropriate fraction thereof. The pharmaceutical compositions and formulations comprising NRH, NARH or a reduced derivative thereof described herein can be used to treat any disorders and conditions, not only the disorders and conditions described herein. For purposes of the content of a pharmaceutical composition, the term “active ingredient”, “active agent”, “therapeutic agent” or “drug” encompasses a prodrug.

A pharmaceutical composition contains NRH, NARH or a reduced derivative thereof (e.g., that of Formula I) in substantially pure form. In some embodiments, the purity of NRH, NARH or a reduced derivative thereof is at least about 95%, 96%, 97%, 98% or 99%. In addition, a pharmaceutical composition is substantially free of contaminants or impurities. In some embodiments, the level of contaminants or impurities other than residual solvent in a pharmaceutical composition is no more than about 5%, 4%, 3%, 2% or 1% relative to the combined weight of the intended active and inactive ingredients.

Pharmaceutical compositions/formulations can be prepared in sterile form. For example, pharmaceutical compositions/formulations for parenteral (e.g., intravenous, subcutaneous or intramuscular) administration by injection or infusion generally are sterile. Sterile pharmaceutical compositions/formulations are compounded or manufactured according to pharmaceutical-grade sterilization standards known to those of skill in the art, such as those disclosed in or required by the United States Pharmacopeia Chapters 797, 1072 and 1211, and 21 Code of Federal Regulations 211.

Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils such as olive oil and sesame oil), aqueous solvents {e.g., saline, buffered saline (e.g., phosphate-buffered saline [PBS]) and isotonic solutions (e.g., Ringer's solution)}, and organic solvents (e.g., dimethyl sulfoxide [DMSO] and alcohols [e.g., ethanol, glycerol and propylene glycol]). Except insofar as any conventional excipient or carrier is incompatible with the active ingredient, the disclosure encompasses the use of conventional excipients and carriers in formulations containing NRH, NARH or a reduced derivative thereof (e.g., that of Formula I). See, e.g., Remington: The Science and Practice of Pharmacy, 21st Ed., Lippincott Williams & Wilkins (Philadelphia, Pa.) (2005); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Fla.) (2004).

Appropriate formulation can depend on various factors, such as the route of administration chosen. Potential routes of administration of pharmaceutical compositions containing NRH, NARH or a reduced derivative thereof (e.g., that of Formula I) include without limitation oral, parenteral (including intradermal, subcutaneous, intramuscular, intravascular, intravenous, intra-arterial, intraperitoneal, intracavitary, intramedullary, intrathecal and topical), and topical (including dermal/epicutaneous, transdermal, mucosal, transmucosal, intranasal [e.g., by nasal spray or drop], ocular [e.g., by eye drop], pulmonary [e.g., by oral or nasal inhalation], buccal, sublingual, rectal [e.g., by suppository], and vaginal [e.g., by suppository]). Topical formulations can be designed to produce a local or systemic therapeutic effect.

As an example, formulations of NRH, NARH or a reduced derivative thereof (e.g., that of Formula I) suitable for oral administration can be presented as, e.g., boluses; capsules (including push-fit capsules and soft capsules), tablets, pills, cachets or lozenges; as powders or granules; as semisolids, electuaries, pastes or gels; as solutions or suspensions in an aqueous liquid or/and a non-aqueous liquid; or as oil-in-water liquid emulsions or water-in-oil liquid emulsions.

Push-fit capsules or two-piece hard gelatin capsules can contain NRH, NARH or a reduced derivative thereof in admixture with, e.g., a filler or inert solid diluent (e.g., calcium carbonate, calcium phosphate, kaolin or lactose), a binder (e.g., a starch), a glidant or lubricant (e.g., talc or magnesium stearate), and a disintegrant (e.g., crospovidone), and optionally a stabilizer or/and a preservative. For soft capsules or single-piece gelatin capsules, NRH, NARH or a reduced derivative thereof can be dissolved or suspended in a suitable liquid (e.g., liquid polyethylene glycol or an oil medium, such as a fatty oil, peanut oil, olive oil or liquid paraffin), and the liquid-filled capsules can contain one or more other liquid excipients or/and semi-solid excipients, such as a stabilizer or/and an amphiphilic agent (e.g., a fatty acid ester of glycerol, propylene glycol or sorbitol).

Tablets can contain NRH, NARH or a reduced derivative thereof in admixture with, e.g., a filler or inert diluent (e.g., calcium carbonate, calcium phosphate, lactose, mannitol or microcrystalline cellulose [MCC]), a binding agent (e.g., a starch, gelatin, acacia, alginic acid or a salt thereof, or MCC), a lubricating agent (e.g., stearic acid, magnesium stearate, talc or silicon dioxide), and a disintegrating agent (e.g., crospovidone, croscarmellose sodium or colloidal silica), and optionally a surfactant (e.g., sodium lauryl sulfate). The tablets can be uncoated or can be coated with, e.g., an enteric coating (e.g., Opadry© Enteric [94 Series]) that protects the active ingredient from the acidic environment of the stomach, or/and with a material that delays disintegration and absorption of the active ingredient in the gastrointestinal (GI) tract and thereby provides a sustained action over a longer time period.

Compositions for oral administration can also be formulated as solutions or suspensions in an aqueous liquid or/and a non-aqueous liquid, or as oil-in-water liquid emulsions or water-in-oil liquid emulsions. Dispersible powder or granules of NRH, NARH or a reduced derivative thereof can be mixed with any suitable combination of an aqueous liquid, an organic solvent or/and an oil and any suitable excipients (e.g., any combination of a dispersing agent, a wetting agent, a suspending agent, an emulsifying agent or/and a preservative) to form a solution, suspension or emulsion.

NRH, NARH and reduced derivatives thereof (e.g., those of Formula I) can also be formulated for parenteral administration by, e.g., injection or infusion to circumvent GI absorption and first-pass metabolism. An exemplary parenteral route is intravenous. Additional advantages of intravenous administration include direct administration of a therapeutic agent into systemic circulation to achieve a rapid systemic effect, and the ability to administer the agent continuously or/and in a large volume if desired. Formulations for injection or infusion can be in the form of, e.g., solutions, suspensions or emulsions in oily or aqueous vehicles, and can contain excipients such as suspending agents, dispersing agents or/and stabilizing agents. For example, aqueous (e.g., saline) or non-aqueous (e.g., oily) sterile injection solutions can contain NRH, NARH or a reduced derivative thereof (e.g., that of Formula I) along with excipients such as an antioxidant, a buffer, a bacteriostat and solutes that render the formulation isotonic with the blood of the subject. Aqueous or non-aqueous sterile suspensions can contain NRH, NARH or a reduced derivative thereof along with excipients such as a suspending agent and a thickening agent, and optionally a stabilizer and an agent that increases the solubility of NRH, NARH or a reduced derivative thereof to allow for the preparation of a more concentrated solution or suspension. As another example, a sterile aqueous solution for injection or infusion (e.g., intravenously or subcutaneously) can contain NRH, NARH or a reduced derivative thereof, sodium chloride, a buffering agent (e.g., sodium citrate), a preservative (e.g., meta-cresol), and optionally a base (e.g., NaOH) or/and an acid (e.g., HCl) to adjust pH.

In some embodiments, a pharmaceutical composition comprising NRH, NARH or a reduced derivative thereof (e.g., that of Formula I) and one or more pharmaceutically acceptable excipients or carriers is in a lyophilized (freeze-dried) form. In some embodiments, the one or more excipients or carriers comprise an amino acid (e.g., glycine or alanine) or/and a stabilizing agent (sucrose, maltose, trehalose or lactose, or any combination thereof), and optionally a bulking agent (e.g., mannitol, dextrose, lactose, sucrose, dextran, trehalose, microcrystalline cellulose, hydroxyethyl starch or glycine, or any combination thereof). In further embodiments, NRH, NARH or a reduced derivative thereof is mixed, dissolved or suspended in an aqueous buffer (e.g., Na₂HPO₄/NaCl) having a pH of about 7.4-10.5, 8-10.5 or 9-10.5 prior to lyophilization. In still further embodiments, the aqueous mixture, solution or suspension comprising NRH, NARH or a reduced derivative thereof is sterilized by filtration through a membrane having a pore size of no more than about 0.2 micron prior to lyophilization. In some embodiments, the lyophilized composition is stored in a hermetically sealed, colored vial or ampule made of glass or plastic (e.g., polyethylene, polypropylene, polyvinyl chloride or polyether ether ketone). In further embodiments, the vial or ampule is under vacuum or under an inert gas (e.g., nitrogen or argon). In still further embodiments, the vial or ampule is stored at reduced temperature (e.g., at about 0-10° C. or 2-8° C.), and with a desiccant (e.g., silica gel) or/and at reduced humidity (e.g., no more than about 40% humidity).

In some embodiments, the lyophilized composition comprising NRH, NARH or a reduced derivative thereof is reconstituted as an aqueous mixture, solution or suspension having a pH of about 7.4-10.5, 8-10.5 or 9-10.5 prior to parenteral (e.g., intravenous, subcutaneous or intramuscular) administration (e.g., injection or infusion). In further embodiments, if the reduced derivative of NRH or NARH has low solubility in water, the lyophilized composition is mixed, dissolved or suspended in a suitable organic solvent (e.g., DMSO) and then diluted with an aqueous solution for reconstitution of the composition. In certain embodiments, the reconstituted, aqueous mixture, solution or suspension comprises Na₂HPO₄ and NaCl, is isotonic, and has a pH of about 8-10.5 or 9-10.5. In additional embodiments, the reconstituted, aqueous mixture, solution or suspension comprises NRH, NARH or a reduced derivative thereof in a concentration of about 1-500 mg/mL, 1-300 mg/mL, 1-200 mg/mL, 1-100 mg/mL, 100-200 mg/mL or 200-300 mg/mL, or about 1-25 mg/mL, 25-50 mg/mL or 50-100 mg/mL.

In some embodiments, a composition for parenteral (e.g., intravenous) administration comprises a complex of NRH, NARH or a reduced derivative thereof (e.g., that of Formula I) with a dendrimer [e.g., a poly(amidoamine) (PAMAM) or/and poly(ethylene glycol) (PEG) dendrimer], which can be, e.g., in an aqueous solution or a colloidal liposomal formulation. As an illustrative example, NRH, NARH or a reduced derivative thereof can be combined with a dendrimer (e.g., a PAMAM or/and PEG dendrimer) by encapsulation (e.g., the dendrimer forms a nanoparticle or micelle encapsulating NRH, NARH or a reduced derivative thereof), electrostatic or ionic interaction or other non-covalent association, or covalent conjugation using, e.g., an enzyme-cleavable linker (e.g., Gly-Phe-Leu-Gly). The dendrimer can optionally have one or more (e.g., ten or more) moieties (e.g., attached to the surface of a dendrimer core) that target the dendrimer-NRH, NARH or a reduced derivative thereof complex to specific organ(s), tissue(s), cell type(s) or organelle(s), such as the liver or mitochondria. For example, the dendrimer can optionally have one or more N-acetylgalactosamine (GalNAc) moieties, which can target the dendrimer-containing composition to the liver by binding to asialoglycoprotein receptors on hepatocytes for treatment of, e.g., a liver or metabolic disorder. Such a dendrimer-containing composition can also be formulated for oral administration or other modes of parenteral administration (e.g., subcutaneous, intramuscular, intrathecal or topical).

For topical administration, NRH, NARH or a reduced derivative thereof (e.g., that of Formula I) can be formulated as, e.g., a buccal or sublingual tablet or pill. Advantages of a buccal or sublingual tablet or pill include avoidance of GI absorption and first-pass metabolism, and rapid absorption into systemic circulation. A buccal or sublingual tablet or pill can be designed to provide faster release of NRH, NARH or a reduced derivative thereof for more rapid uptake into systemic circulation. A buccal or sublingual tablet or pill can contain suitable excipients, including without limitation any combination of fillers and diluents (e.g., mannitol and sorbitol), binding agents (e.g., sodium carbonate), wetting agents (e.g., sodium carbonate), disintegrants (e.g., crospovidone and croscarmellose sodium), lubricants (e.g., silicon dioxide [including colloidal silicon dioxide] and sodium stearyl fumarate), stabilizers (e.g., sodium bicarbonate), flavoring agents (e.g., spearmint flavor), sweetening agents (e.g., sucralose), and coloring agents (e.g., yellow iron oxide).

For topical administration, NRH, NARH or a reduced derivative thereof (e.g., that of Formula I) can also be formulated for intranasal administration. The nasal mucosa provides a big surface area, a porous endothelium, a highly vascular subepithelial layer and a high absorption rate, and hence allows for high bioavailability. Moreover, intranasal administration avoids first-pass metabolism and can introduce a significant concentration of the active ingredient to the central nervous system (CNS). An intranasal formulation can comprise NRH, NARH or a reduced derivative thereof along with excipients, such as a solubility enhancer (e.g., propylene glycol), a humectant (e.g., mannitol or sorbitol), a buffer and water, and optionally a preservative (e.g., benzalkonium chloride), a mucoadhesive agent (e.g., hydroxyethylcellulose) or/and a penetration enhancer. An intranasal solution or suspension formulation can be administered to the nasal cavity by any suitable means, including but not limited to a dropper, a pipette, or spray using, e.g., a metering atomizing spray pump.

An additional mode of topical administration of NRH, NARH or a reduced derivative thereof (e.g., that of Formula I) is pulmonary, including by oral inhalation and nasal inhalation. The lungs serve as a portal to the systemic circulation. Advantages of pulmonary drug delivery include, for example: 1) avoidance of first-pass hepatic metabolism; 2) fast drug action; 3) large surface area of the alveolar region for absorption, high permeability of the lungs (thin air-blood barrier), and profuse vasculature of the airways; 4) reduced extracellular enzyme levels compared to the GI tract due to the large alveolar surface area; and 5) smaller doses to achieve equivalent therapeutic effect compared to other oral routes, and hence reduced systemic side effects. Oral inhalation can also enable more rapid action of a drug in the CNS. An advantage of oral inhalation over nasal inhalation includes deeper penetration/deposition of the drug into the lungs. Oral or nasal inhalation can be achieved by means of, e.g., a metered-dose inhaler, a dry powder inhaler or a nebulizer, as is known in the art. In certain embodiments, a sterile aqueous solution for oral inhalation contains NRH, NARH or a reduced derivative thereof, sodium chloride, a buffering agent (e.g., sodium citrate), optionally a preservative (e.g., meta-cresol), and optionally a base (e.g., NaOH) or/and an acid (e.g., HCl) to adjust pH.

Topical formulations for application to the skin or mucosa can be useful for transdermal or transmucosal administration of a drug into the underlying tissue or/and the blood for systemic distribution. Advantages of topical administration can include circumvention of GI absorption and first-pass metabolism, delivery of a drug with a short half-life and low oral bioavailability, more controlled and sustained release of the drug, a more uniform plasma dosing or delivery profile of the drug, less frequent dosing of the drug, less side effects, minimal or no invasiveness, ease of self-administration, and increased patient compliance.

In general, compositions suitable for topical administration include without limitation liquid or semi-liquid preparations such as sprays, gels, liniments and lotions, oil-in-water or water-in-oil emulsions such as creams, foams, ointments and pastes, and solutions or suspensions such as drops (e.g., eye drops, nose drops and ear drops). In some embodiments, a topical composition comprises a drug dissolved, dispersed or suspended in a carrier. The carrier can be in the form of, e.g., a solution, a suspension, an emulsion, an ointment or a gel base, and can contain, e.g., petrolatum, lanolin, a wax (e.g., bee wax), mineral oil, a long-chain alcohol, polyethylene glycol or polypropylene glycol, or a diluent (e.g., water or/and an alcohol [e.g., ethanol or propylene glycol]), or any combination thereof. A solvent such as an alcohol can be used to solubilize the drug. A topical composition can contain any of a variety of excipients, such as a gelling agent, an emulsifier, a thickening agent, a buffer, a stabilizer, an antioxidant, a preservative, a chemical permeation enhancer (CPE) or an irritation-mitigating agent, or any combination thereof. A topical composition can include, or a topical formulation can be administered by means of, e.g., a transdermal or transmucosal delivery device, such as a transdermal patch, a microneedle patch or an iontophoresis device. A topical composition can deliver a drug transdermally or transmucosally via a concentration gradient (with or without the use of a CPE) or an active mechanism (e.g., iontophoresis or microneedles).

For transdermal or transmucosal administration, in some embodiments a topical composition comprises a chemical penetration enhancer (CPE) that increases permeation of a drug across the skin or mucosa into the underlying tissue or/and systemic circulation. Examples of CPEs include without limitation alcohols and fatty alcohols (e.g., methanol, ethanol, isopropyl alcohol, pentanol, lauryl alcohol, oleyl alcohol, menthol, benzyl alcohol, diethylene glycol mono-ethyl ether, propylene glycol, dipropylene glycol, polyethylene glycol and glycerol); ethers (e.g., eucalyptol); fatty acids (e.g., capric acid, lauric acid, myristic acid, oleic acid, linoleic acid and linolenic acid); esters, fatty alcohol esters and fatty acid esters (e.g., ethyl acetate, methyl laurate, isopropyl myristate, isopropyl palmitate, methyl oleate, ethyl oleate, propylene glycol mono-oleate, glycerol mono-oleate, triacetin and pentadecalactone); hydroxyl-containing esters, fatty alcohol esters and fatty acid esters (e.g., lauryl lactate, glyceryl/glycerol monolaurate, glycerol monoleate [mono-olein], sorbitan oleate, octyl salicylate and fatty acid esters of saccharides [e.g., sucrose fatty acid esters such as sucrose laurate]); amides, fatty amine amides and fatty acid amides (e.g., urea, dimethylformamide, dimethylacetamide, diethylacetamide, diethyltoluamide, N-lauroyl sarcosine, 1-dodecylazacycloheptane-2-one [laurocapram or Azone®], Azone-related compounds, and pyrrolidone compounds [e.g., 2-pyrrolidone and N-methyl-2-pyrrolidone]); and ionic and non-ionic surfactants (e.g., cetyltrimethylammonium bromide, sodium laurate, sodium laureth sulfate [sodium lauryl ether sulfate], sodium cholate, sodium lauroyl sarcosinate, N-lauroyl sarcosine, sorbitan monolaurate, Brij® surfactants, Pluronic® surfactants, Tween® surfactants, saponins, alkyl glycosides, and fatty ether and fatty ester saccharides). US 2007/0269379 provides an extensive list of CPEs.

In some embodiments, the CPE includes a surfactant. In certain embodiments, the CPE includes two or more surfactants, such as a non-ionic surfactant (e.g., sorbitan monolaurate or N-lauroyl sarcosine) and an ionic surfactant (e.g., an anionic surfactant such as sodium lauroyl sarcosinate). In other embodiments, the CPE includes a surfactant (e.g., an anionic surfactant such as sodium laureth sulfate) and an aromatic compound (e.g., 1-phenylpiperazine). Such combinations of CPEs can greatly enhance permeation of a drug through the skin with a low potential for skin irritation.

For transmucosal administration, in certain embodiments the CPE is or includes an alkyl glycoside (e.g., a 1-O or S—C₈-C₂₀ alkyl glycoside such as the corresponding glucoside, galactoside, mannoside, lactoside, maltoside [e.g., dodecyl, tridecyl or tetradecyl maltoside], melibioside or sucroside [e.g., dodecyl sucrose]), or a fatty ether or fatty ester saccharide (e.g., a C₈-C₂₀ alkyl ether or ester saccharide such as the corresponding glucoside, galactoside, mannoside, lactoside, maltoside, melibioside, sucroside [e.g., sucrose mono-, di- and tri-dodecanoate and mixtures thereof such as J-1205 and J-1216] or trehaloside).

In some embodiments, NRH, NARH or a reduced derivative thereof (e.g., that of Formula I) is administered via a transdermal patch. In certain embodiments, a transdermal patch is a reservoir-type patch comprising an impermeable backing layer/film, a liquid- or gel-based drug reservoir, a semi-permeable membrane that controls drug release, and a skin-contacting adhesive layer. The semi-permeable membrane can be composed of, e.g., a suitable polymeric material such as cellulose nitrate or acetate, polyisobutene, polypropylene, polyvinyl acetate or a polycarbonate. In other embodiments, a transdermal patch is a drug-in-adhesive patch comprising an impermeable backing layer/film and a skin-contacting adhesive layer incorporating the drug in a polymeric or viscous adhesive. The adhesive of the drug-loaded, skin-contacting adhesive layer can be, e.g., a pressure-sensitive adhesive (PSA), such as a PSA composed of an acrylic polymer (e.g., polyacrylate), a polyalkylene (e.g., polyisobutylene) or a silicone-based polymer (e.g., silicone-2675 or silicone-2920). Transdermal drug-delivery systems, including patches, can be designed to provide controlled and prolonged release of a drug over a period of about 1 week, 2 weeks, 3 weeks, 1 month or longer.

In some embodiments, NRH, NARH or a reduced derivative thereof (e.g., that of Formula I) is delivered from a sustained-release composition. As used herein, the term “sustained-release composition” encompasses sustained-release, prolonged-release, extended-release, delayed-release and slow-release compositions, systems and devices. A sustained-release composition can also be designed to be controlled-release. Advantages of a sustained-release composition include without limitation a more uniform blood level of the drug (e.g., avoidance of wide peak-to-trough fluctuations), delivery of a therapeutically effective amount of the drug over a prolonged time period, reduced frequency of administration, and reduced side effects (e.g., avoidance of a drug overdose). In certain embodiments, a sustained-release composition delivers NRH, NARH or a reduced derivative thereof over a period of at least about 12 hours, 1 day, 2 days, 3 days, 1 week, 2 weeks, 3 weeks, 1 month, 2 months, 3 months or longer. In some embodiments, a sustained-release composition is a drug-encapsulation system, such as nanoparticles, microparticles or a capsule made of, e.g., a lipid, a biodegradable polymer or/and a hydrogel. In certain embodiments, a sustained-release composition comprises a hydrogel. Non-limiting examples of polymers of which a hydrogel can be composed include polyvinyl alcohol, acrylate polymers (e.g., sodium polyacrylate), and other homopolymers and copolymers having a relatively large number of hydrophilic groups (e.g., hydroxyl or/and carboxylate groups). In other embodiments, a sustained-release drug-encapsulation system comprises a membrane-enclosed reservoir, wherein the reservoir contains a drug and the membrane is permeable to the drug. Such a drug-delivery system can be in the form of, e.g., a transdermal patch.

In certain embodiments, a sustained-release composition is an oral dosage form, such as a tablet or capsule. For example, a drug can be embedded in an insoluble porous matrix such that the dissolving drug must make its way out of the matrix before it can be absorbed through the GI tract. Alternatively, a drug can be embedded in a matrix that swells to form a gel through which the drug exits. Sustained release can also be achieved by way of a single-layer or multi-layer osmotic controlled-release oral delivery system (OROS). An OROS is a tablet with a semi-permeable outer membrane and one or more small laser-drilled holes in it. As the tablet passes through the body, water is absorbed through the semi-permeable membrane via osmosis, and the resulting osmotic pressure pushes the drug out through the hole(s) in the tablet and into the GI tract where it can be absorbed.

In further embodiments, a sustained-release composition is formulated as polymeric nanoparticles or microparticles, which can be delivered, e.g., by injection or inhalation or as an implant (e.g., a depot). In some embodiments, the polymeric implant or polymeric nanoparticles or microparticles are composed of a biodegradable polymer. In certain embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. For instance, biodegradable polymeric nano-/microspheres composed of polylactic acid or/and polyglycolic acid can serve as sustained-release pulmonary drug-delivery systems. The biodegradable polymer of the polymeric implant or polymeric nanoparticles or microparticles can be selected so that the polymer substantially completely degrades around the time the period of treatment is expected to end, and so that the byproducts of the polymer's degradation, like the polymer, are biocompatible.

In some embodiments, a sustained-release composition comprises a water-soluble polymer [e.g., poly(DL-lactide)] encapsulating NRH, NARH or a reduced derivative thereof (e.g., that of Formula I) complexed with or conjugated to a dendrimer (e.g., a PAMAM or/and PEG dendrimer). In other embodiments, a sustained-release composition is a nanoparticle composed of a dendrimer (e.g., a PAMAM or/and PEG dendrimer) and encapsulating NRH, NARH or a reduced derivative thereof. The dendrimer (e.g., the surface of a nanoparticle composed of a dendrimer) can optionally have or bear one or more moieties for targeting to specific organ(s), tissue(s), cell type(s) or organelle(s), such as one or more N-acetylgalactosamine (GalNAc) moieties for targeting to the liver for treatment of, e.g., a liver or metabolic disorder. A dendrimer can have good cell membrane permeability.

In other embodiments, a sustained-release composition is in the form of nanoparticles or microparticles composed of one or more lipids (e.g., solid lipid nanoparticles [SLNs]) and encapsulating NRH, NARH or a reduced derivative thereof (e.g., that of Formula I). The one or more lipids composing the nanoparticles or microparticles (e.g., the lipid core of SLNs) can be, e.g., physiological lipid(s) (thereby avoiding biotoxicity) and can be selected from, e.g., triglycerides (e.g. tristearin and Miglyol® 812), diglycerides (e.g. glycerol behenate), monoglycerides (e.g. glycerol monostearate), fatty acids (e.g. stearic acid), steroids (e.g. cholesterol), and waxes (e.g. cetyl palmitate). The lipid core of SLNs can be stabilized by one or more surfactants or emulsifiers. Lipid nanoparticles or microparticles can incorporate a lipophilic or hydrophilic drug. For example, a lipid core composed of stearic acid can incorporate a hydrophilic drug in SLNs. Relatively slow or slow degradation of the lipid(s) can provide controlled, slow or sustained release of NRH, NARH or a reduced derivative thereof. Furthermore, the lipid nanoparticles or microparticles can increase the oral bioavailability of the drug by improving gastrointestinal absorption, can increase penetration of the drug into cells (including target cells) after oral or parenteral administration by improving cell membrane permeability, and can increase the stability and half-life of the drug by protecting the drug from the chemical environments and degradative enzymes of the body. The lipid nanoparticles or microparticles can be conjugated to a polymer, such as a hydrophilic polymer (e.g., PEG) to increase the aqueous solubility of the lipid particles. Moreover, the lipid nanoparticles or microparticles can be conjugated to one or more targeting moieties, such as one or more GalNAc moieties for targeting to the liver for treatment of, e.g., a liver or metabolic disorder.

For a delayed or sustained release of NRH, NARH or a reduced derivative thereof (e.g., that of Formula I), a composition can also be formulated as a depot that can be implanted in or injected into a subject, e.g., intramuscularly, intracutaneously or subcutaneously. A depot formulation can be designed to deliver NRH, NARH or a reduced derivative thereof over a longer period of time, e.g., over a period of at least about 1 week, 2 weeks, 3 weeks, 1 month, 6 weeks, 2 months, 3 months or longer. For example, NRH, NARH or a reduced derivative thereof can be formulated with a polymeric material (e.g., polyethylene glycol [PEG], polylactic acid [PLA] or polyglycolic acid [PGA], or a copolymer thereof [e.g., PLGA]), with a hydrophobic material (e.g., as an emulsion in an oil) or/and an ion-exchange resin, as a more lipophilic derivative (e.g., as an ester of or a salt with a fatty acid such as a C₈-C₂₀ fatty acid [e.g., decanoic acid]), or as a sparingly soluble derivative (e.g., a sparingly soluble salt). A depot can also be formed from liposomes, micelles, cholestosomes, nano-/microparticles or nano-/microspheres encapsulating NRH, NARH or a reduced derivative thereof. As an illustrative example, NRH, NARH or a reduced derivative thereof can be incorporated or embedded in sustained-release nano-/microparticles composed of PLGA and formulated as a monthly depot.

In some embodiments, a pharmaceutical composition containing NRH, NARH or a reduced derivative thereof (e.g., that of Formula I) is a controlled-release composition. A controlled-release composition can deliver a drug in a controlled time-dependent manner, and can be designed to deliver the drug, e.g., with delay after administration or/and for a prolonged time period. A controlled-release composition can also be designed to achieve particular profiles of dissolution of the drug in particular environments (e.g., in the GI tract) and to improve pharmacokinetics (e.g., bioavailability) of the drug. In certain embodiments, a controlled-release composition is administered once daily, once every two or three days, twice weekly or once weekly. In certain embodiments, a controlled-release composition is enterically coated for oral administration.

In some embodiments, a capsule for oral administration contains a plurality of pellets, each pellet comprising a pellet core containing NRH, NARH or a reduced derivative thereof (e.g., that of Formula I) and a controlled-release coating surrounding the pellet core. NRH, NARH or a reduced derivative thereof can be, e.g., dispersed in a solid or semi-solid pellet core or in a drug layer coating the pellet core. In certain embodiment, the controlled-release coating comprises a polymer such as ethyl cellulose or/and hydroxypropyl cellulose, optionally povidone or/and hydroxypropyl methyl cellulose, and optionally a plasticizer (e.g., dibutyl sebacate).

In addition, pharmaceutical compositions comprising NRH, NARH or a reduced derivative thereof (e.g., that of Formula I) can be formulated as, e.g., liposomes, micelles, cholestosomes, nano-/microparticles or nano-/microspheres encapsulating the drug, whether or not designed for controlled, slow or sustained release. The nano-/microparticles or nano/-microspheres can be composed of, e.g., a lipid, a biodegradable polymer or/and a non-degradable polymer, or a hydrogel. For example, liposomes can be used as a sustained-release pulmonary drug-delivery system that delivers a drug to the alveolar surface for treatment of a lung disorder or a systemic disorder. Such liposomes, micelles, cholestosomes, nano-/microparticles and nano-/microspheres can be formulated for oral or parenteral (e.g., intravenous, subcutaneous, intramuscular, intrathecal or topical) administration.

In some embodiments, liposomes or micelles are composed of one or more phospholipids. Phospholipids include without limitation phosphatidic acids (e.g., DEPA, DLPA, DMPA, DOPA, DPPA and DSPA), phosphatidylcholines (e.g., DDPC, DEPC, DLPC, DLOPC, DMPC, DOPC, DPPC, DSPC, MPPC, MSPC, PLPC, PMPC, POPC, PSPC, SMPC, SOPC and SPPC), phosphatidylethanolamines (e.g., DEPE, DLPE, DMPE, DOPE, DPPE, DSPE and POPE), phosphatidylglycerols (e.g., DEPG, DLPG, DMPG, DOPG, DPPG, DSPG and POPG), phosphatidylserines (e.g., DLPS, DMPS, DOPS, DPPS and DSPS), and salts (e.g., sodium and ammonium salts) thereof. In certain embodiments, liposomes or micelles are composed of one or more phosphatidylcholines. Liposomes have a hydrophilic core, so liposomes are particularly suited for delivery of more hydrophilic drugs, whereas micelles have a hydrophobic core, so micelles are particularly suited for delivery of more hydrophobic drugs. Liposomes and micelles can permeate across biological membranes. Liposomes and micelles composed of a fusogenic lipid (e.g., DPPG) can fuse with the plasma membrane of cells and thereby deliver a drug into those cells. Liposomes and micelles can provide controlled, slow or sustained release of a drug based in part on the rate of extracellular degradation of the liposomes and micelles.

In other embodiments, micelles are composed of biodegradable natural or/and synthetic polymer(s), such as lactosomes. In certain embodiments, micelles are lactosomes composed of a block copolymer, such as that containing two or three poly(sarcosine) blocks and a poly(lactic acid) block, where lactic acid can be L-lactic acid, D-lactic acid or D,L-lactic acid. In further embodiments, micelles are composed of an amphiphilic block copolymer, such as an amphiphilic di-, tri- or tetra-block copolymer containing hydrophilic block(s) and hydrophobic block(s). In additional embodiments, micelles are composed of one or more surfactants.

Cholestosomes are lipid particles (e.g., nanoparticles or microparticles) composed of one or more naturally occurring (and thus non-toxic) lipids or/and lipid esters and encapsulating a drug. They are typically neutral. Orally administered cholestosomes are resistant to degradation in the stomach, are absorbed through the intestines into the bloodstream (or into the lymphatic system if incorporated into chylomicrons), are taken up by cells (e.g., via endocytosis or permeation), escape lysosomal trapping, and degrade in the cells to release the drug. Cholestosomes can provide controlled, slow or sustained release of the drug based in part on the rate of extracellular degradation of the cholestosomes.

In some embodiments, NRH, NARH or a reduced derivative thereof (e.g., that of Formula I) is encapsulated in nano-/microparticles or nano-/microspheres composed of a biodegradable synthetic or natural polymer, such as PLA, PGA, PLGA, poly(F-caprolactone) (PCL) or a polysaccharide (e.g., chitosan), where lactic acid can be L-lactic acid, D-lactic acid or D,L-lactic acid. In other embodiments, NRH, NARH or a reduced derivative thereof (e.g., that of Formula I) is encapsulated in nano-/microparticles or nano-/microspheres composed of a substantially non-degradable polymer, such as PEG. In further embodiments, NRH, NARH or a reduced derivative thereof (e.g., that of Formula I) is encapsulated in nano-/microparticles or nano-/microspheres composed of a mixture or blend of a biodegradable polymer (e.g., PLA, PGA, PLGA or PCL) and a substantially non-degradable polymer (e.g., PEG). In still further embodiments, NRH, NARH or a reduced derivative thereof (e.g., that of Formula I) is encapsulated in nano-/microparticles or nano-/microspheres composed of a copolymer or block copolymer containing a biodegradable polymer (e.g., PLA, PGA, PLGA or PCL) and a substantially non-degradable polymer (e.g., PEG). In yet further embodiments, NRH, NARH or a reduced derivative thereof (e.g., that of Formula I) is encapsulated in nano-/microparticles or nano-/microspheres composed of a dendrimer, such as a PAMAM or/and PEG dendrimer. Such compositions can provide controlled, slow or sustained release of NRH, NARH or a reduced derivative thereof based in part on the rate of degradation of the polymer or dendrimer or/and the rate of diffusion of the drug through the polymer or dendrimer (e.g., through pores formed by the polymer or dendrimer).

In some embodiments, liposomes, micelles, cholestosomes, nano-/microparticles or nano-/microspheres encapsulating NRH, NARH or a reduced derivative thereof (e.g., that of Formula I) are conjugated to or coated with a biodegradable or non-degradable polymer. In certain embodiments, the surface-conjugating/coating polymer is a hydrophilic polymer, such as PEG. In some embodiments, the surface-conjugating/coating polymer (e.g., PEG) has a molecular weight of about 0.5-1 kDa, 1-2 kDa, 2-5 kDa or higher. Conjugation or coating of the surface of such compositions with a polymer can have various benefits, including minimizing aggregation and immunogenicity of the compositions, and shielding the compositions from the degradative environments of the body, opsonization and phagocytosis, thereby increasing their half-life.

In further embodiments, liposomes, micelles, cholestosomes, nano-/microparticles or nano-/microspheres encapsulating NRH, NARH or a reduced derivative thereof (e.g., that of Formula I) are conjugated to one or more targeting moieties. In certain embodiments, the targeting moieties are GalNAc moieties for targeting of the compositions to the liver for treatment of, e.g., a liver or metabolic disorder.

Pharmaceutical compositions can be manufactured in any suitable manner known in the art, such as by means of conventional mixing, dissolving, suspending, granulating, dragee-making, levigating, emulsifying, encapsulating, entrapping or compressing processes, or any combination thereof.

A pharmaceutical composition can be presented in unit dosage form as a single dose wherein all active and inactive ingredients are combined in a suitable system, and components do not need to be mixed to form the composition to be administered. A unit dosage form generally contains a therapeutically effective dose of the drug, but can contain an appropriate fraction thereof so that taking multiple unit dosage forms achieves the therapeutically effective dose. Representative examples of a unit dosage form include a tablet, capsule or pill for oral uptake; a solution in a pre-filled syringe of a single-use pen or a pen with a dose counter for parenteral (e.g., intravenous, subcutaneous or intramuscular) injection; a capsule, cartridge or blister pre-loaded in or manually loaded into an inhaler; and a reservoir-type transdermal patch or a drug-in-adhesive patch.

Alternatively, a pharmaceutical composition can be presented as a kit in which the drug, excipient(s) and carrier(s) [e.g., solvent(s)] are provided in two or more separate containers (e.g., ampules, vials, tubes, bottles or syringes) and need to be combined to form the composition to be administered. The kit can contain instructions for storing, preparing and administering the composition (e.g., a solution to be injected or infused parenterally).

A kit can contain all active and inactive ingredients in unit dosage form or the active ingredient and inactive ingredients in two or more separate containers, and can contain instructions for administering or using the pharmaceutical composition to treat a medical condition. A kit can further contain a device for delivering the composition, such as a needle and a syringe, an injection pen, an inhaler or a transdermal patch.

In some embodiments, a kit contains a pharmaceutical composition comprising NRH, NARH or a reduced derivative thereof (e.g., that of Formula I) and one or more pharmaceutically acceptable excipients or carriers in a lyophilized (freeze-dried) or powder form. In some embodiments, the kit further contains:

an aqueous solution for reconstituting the lyophilized or powder composition;

equipment (e.g., a needle and a syringe, an infusion bag or an infusion pump) for parenteral (e.g., intravenous, subcutaneous or intramuscular) administration (e.g., injection or infusion) of the reconstituted composition; and

instructions for preparing and administering the reconstituted composition.

In certain embodiments, the reconstituted, aqueous composition has a pH of about 7.4-10.5, 8-10.5 or 9-10.5. In further embodiments, if the lyophilized or powder composition comprises a reduced derivative of NRH or NARH that has low solubility in water, the kit further contains a suitable organic solvent (e.g., DMSO) and instructions for mixing, dissolving or suspending the reduced derivative of NRH or NARH in the organic solvent and then diluting the organic mixture, solution or suspension with the aqueous solution for reconstitution of the composition. In additional embodiments, the kit further contains instructions for storing the lyophilized or powder composition, such as at reduced temperature (e.g., at about 0-10° C. or 2-8° C.) and with a dessicant (e.g., silica gel) or/and at reduced humidity (e.g., no more than about 40% humidity). In some embodiments, the lyophilized or powder composition is stored in a hermetically sealed, colored vial or ampule made of glass or plastic which is under vacuum or under an inert gas (e.g., nitrogen or argon). In additional embodiments, the kit further contains instructions for administering or using the reconstituted composition to treat any disorder or condition described herein, such as an immune-related disorder (e.g., SIRS or sepsis), a kidney disorder (e.g., AKI or HRS), a liver disorder (e.g., alcoholic hepatitis, ALF, ACLF, cirrhosis or HRS), a hemolytic disorder (e.g., hemolysis or hemolytic anemia), or a disorder or condition associated with oxidative stress, damage or injury (e.g., methemoglobinemia or anemia).

The description and all of the embodiments relating to pharmaceutical compositions and kits comprising NRH, NARH and reduced derivatives thereof also apply to pharmaceutical compositions and kits comprising metabolites of NRH, NARH and reduced derivatives thereof and to pharmaceutical compositions and kits comprising intermediates in the biosynthesis of NADH from NRH or NARH, such as NMNH and NAMNH.

Synthesis of NRH, NARH and Reduced Derivatives Thereof

### Abbreviations:

Boc=tert-butyloxycarbonyl
DCC=N,N′-dicyclohexylcarbodiimide
DCM=dichloromethane
DMAP=4-dimethylaminopyridine
DMF=N,N-dimethylformamide
DMP=2,2-dimethoxypropane
HMDS=hexamethyldisilazide
HOBt=hydroxybenzotriazole
HPLC=high-performance/pressure liquid chromatography
LCMS, LC-MS or LC/MS=liquid chromatography-mass spectrometry
MeOH=methanol
OAc=acetate
p-TSA=para-toluenesulfonic acid
pyr=pyridine
RT=ambient/room temperature
tBuMgCl=tert-butylmagnesium chloride
TBAF=tetrabutylammonium fluoride
TBSCl=tert-butyldimethylsilyl chloride
TEA=triethylamine
TFA=trifluoroacetic acid
THF=tetrahydrofuran
TLC=thin-layer chromatography
TMSCl=trimethylsilyl chloride
TMSOTf=trimethylsilyl trifluoromethanesulfonate

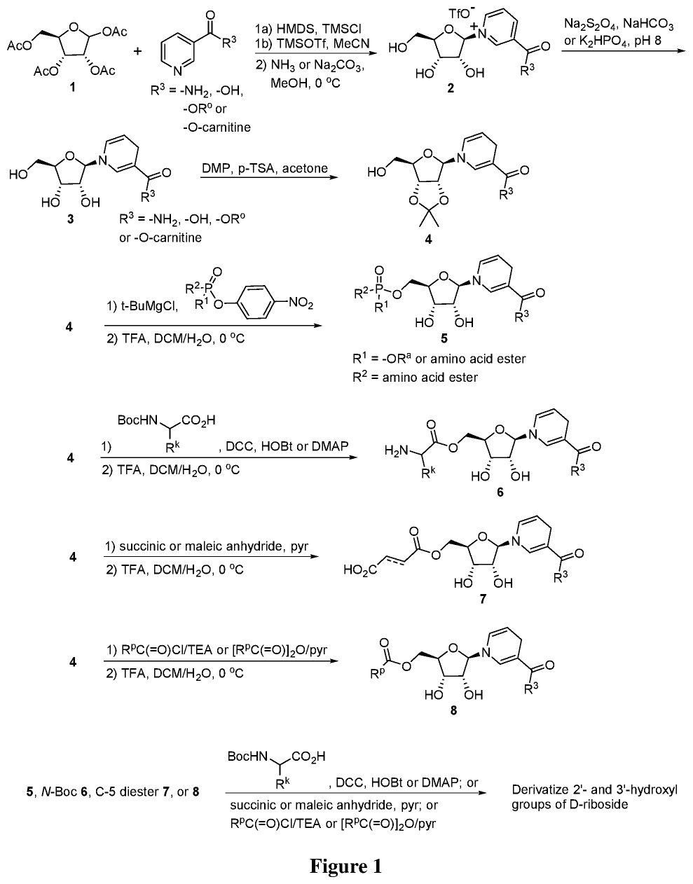

FIG. 1 shows an exemplary process for synthesizing NRH, NARH and reduced derivatives thereof of Formula I which have the 5′-hydroxyl group, and optionally the 2′- and 3′-hydroxyl groups, of D-riboside derivatized. Glycosylation of nicotinamide [R³ can also be —NHRⁿ or —N(Rⁿ)₂], nicotinic acid or a nicotinate ester with commercially available peracetylated β-D-ribofuranose 1 using Vorbruggen's protocol followed by cleavage of the acetate groups under mild basic conditions provides nicotinamide riboside (NR), nicotinic acid riboside (NAR) or nicotinate ester riboside 2. Reduction of the nicotinyl ring with sodium dithionite (sodium hydrosulfite) furnishes dihydronicotinamide riboside (NRH), dihydronicotinic acid riboside (NARH) or dihydronicotinate ester riboside 3. Protection of the 2′- and 3′-hydroxyl groups of D-riboside as an acetonide affords compound 4. Coupling of compound 4 to an activated phosphoramidate or phosphorodiamidate, an N-Boc amino acid, succinic or maleic anhydride, or an acid chloride or anhydride followed by deprotection of the acetonide under acidic conditions yields compound 5, 6, 7 or 8, respectively, derivatized at the 5′-hydroxyl group of D-riboside. Alternatively, phosphorodiamidate 5 can be prepared by first reaction of compound 4 with phosphoryl chloride (POCl₃) at 0° C. followed by addition of at least two equivalents of an amino acid ester and a base (e.g., TEA) at −78° C. and stirring of the resulting mixture at ambient temperature. The maleic acid group of compound 7 can be isomerized to fumaric acid using, e.g., a catalytic amount of a mineral acid (e.g., HCl), a thiourea, or bromine under photolysis conditions. Alternatively, compound 4 can be reacted with commercially available methyl (2E)-4-chloro-4-oxobut-2-enoate (fumaric acid chloride, methyl ester) in the presence of a base (e.g., TEA). The 2′- and 3′-hydroxyl groups of D-riboside can optionally be derivatized by coupling of compound 5, N-Boc compound 6, compound 7 with the succinic acid or maleic/fumaric acid group protected as an ester, or compound 8 to an N-Boc amino acid, succinic or maleic anhydride, or an acid chloride or anhydride.

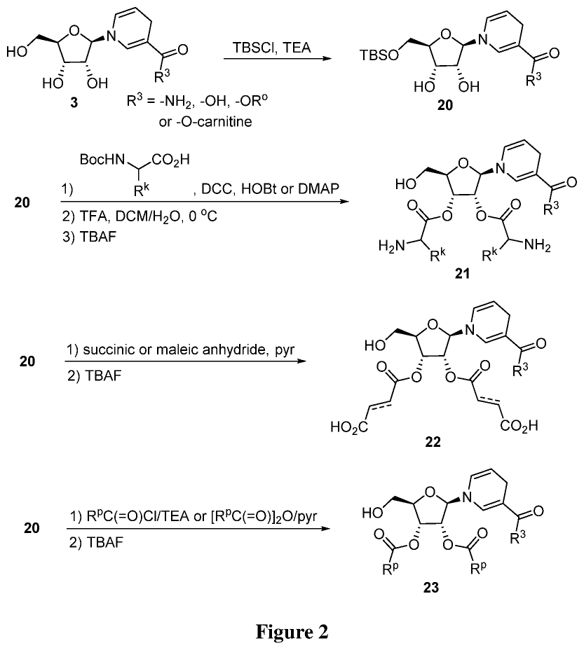

FIG. 2 shows an exemplary process for synthesizing reduced derivatives of NRH and NARH of Formula I which have the 2′- and 3′-hydroxyl groups of D-riboside derivatized. Selective protection of the least sterically hindered 5′-hydroxyl group of NRH, NARH or dihydronicotinate ester riboside 3 with one equivalent of TBSCl affords compound 20. Coupling of compound 20 to an N-Boc amino acid, succinic or maleic anhydride, or an acid chloride or anhydride followed by deprotection of the silyl ether produces compound 21 (after deprotection of the N-Boc groups), 22 or 23, respectively, derivatized at the 2′- and 3′-hydroxyl groups of D-riboside. The maleic acid groups of compound 22 can be isomerized to fumaric acid, or compound 20 can be reacted with fumaric acid chloride, methyl ester in the presence of a base, as described above.

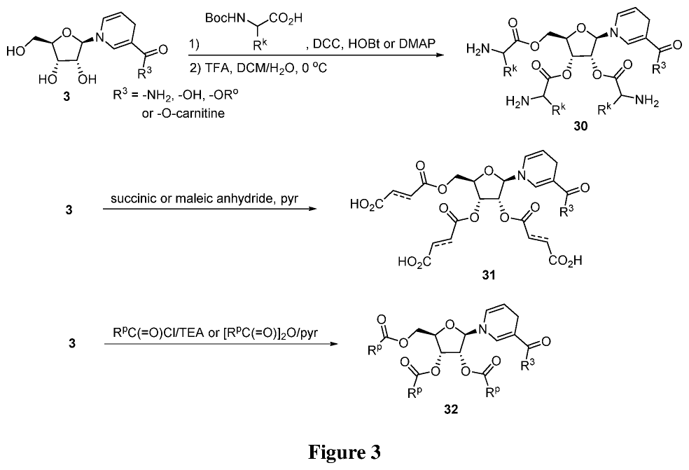

FIG. 3 shows an exemplary process for synthesizing reduced derivatives of NRH and NARH of Formula I which have the 2′-, 3′- and 5′-hydroxyl groups of D-riboside derivatized. Coupling of NRH, NARH or dihydronicotinate ester riboside 3 to an N-Boc amino acid, succinic or maleic anhydride, or an acid chloride or anhydride produces compound 30 (after deprotection of the N-Boc groups), 31 or 32, respectively, derivatized at the 2′-, 3′- and 5′-hydroxyl groups of D-riboside. The maleic acid groups of compound 31 can be isomerized to fumaric acid, or compound 3 can be reacted with fumaric acid chloride, methyl ester in the presence of a base, as described above.

### EXAMPLES

The following examples are intended only to illustrate the disclosure. Other processes, assays, studies, protocols, procedures, methodologies, reagents and conditions may alternatively be used as appropriate.

### Example 1. Synthesis of NRH and NRH-Triacetate

### Synthesis of 3-Carboxamide-N-(2,3,5-tri-O-acetyl-β-D-ribofuranosyl)pyridinium Triflate (NR-Triacetate)

To a solution of nicotinamide (15 g, 0.122 mol) in HMDS (200 mL) was added TMSCl (245 mL, 0.245 mol, 1 M in THF) at RT, and the resulting suspension was refluxed at 120° C. for 3 hr, during which the reaction mixture became clear. After the reaction mixture cooled to RT, removal of solvent in vacuo at 40° C. yielded N,N-bis(trimethylsilyl)nicotinamide (32 g) as an off-white solid. The solid was added to a solution containing 1,2,3,5-tetra-O-acetyl-β-D-ribofuranose (30 g, 0.0942 mol) in 1,2-dichloroethane (460 mL) under N₂ at RT. To the resulting solution was added TMSOTf (100 mL, 0.471 mol) dropwise, and the reaction was stirred at 45° C. for 2 hr. Removal of solvent in vacuo below 40° C. afforded NR-triacetate triflate as a thick syrup, which was purified by slurrying with 20% ethanol in acetone at RT, filtration and suck-drying the filtered material to afford the desired product (110 g).

### Synthesis of N-(2,3,5-tri-O-acetyl-β-D-ribofuranosyl)dihydronicotinamide (NRH-Triacetate)

To a mixture of sodium dithionite (45.5 g) and sodium bicarbonate (54.8 g) in purified water (800 mL, pre-purged with N₂) under N₂ at RT was added a solution of NR-triacetate triflate (69.3 g) in water (261 mL, degassed with N₂) under N₂ through a funnel over a period of 25-30 min at RT. The reaction mixture was stirred with a mechanical stirrer at RT overnight for 16 hr. After completion of the reaction as determined by HPLC, DCM (655 mL) was added, and the resulting mixture was stirred for 15 min. After separation of the bottom organic layer, DCM (1310 mL) was added to the aqueous layer, and the resulting mixture was stirred for 15 min. After separation of the bottom organic layer from the aqueous layer, to the two pooled organic layers was added water (520 mL), and the resulting mixture was stirred for 15 min. The bottom organic layer was separated from the aqueous layer and dried over sodium sulfate. After filtration and concentration of the filtrate in vacuo, n-heptane (400 mL) was added to the concentrate. Removal of solvent and drying in vacuo at 35-40° C. furnished NRH-triacetate (34.6 g and 98% purity by LC-MS), which was stored under N₂.

### Synthesis of N-(β-D-ribofuranosyl)dihydronicotinamide (NRH)

Sodium carbonate (28 g) was added to a mixture of NRH-triacetate (34.6 g) in anhydrous methanol (MeOH, 2.61 L) under N₂ at 0° C., and the reaction mixture was stirred at 0° C. for 2-3 hr. After completion of the reaction, the reaction mixture was filtered, and the filtered material was washed with MeOH (265 mL). The filtrate was concentrated in vacuo below 40° C. The concentrate was diluted with 1:1 MeOH:DCM (2.61 L), and the resulting mixture was stirred for 20-30 min and then filtered through a celite bed. The celite bed was washed with 1:1 MeOH:DCM (660 mL). The filtrate was filtered, concentrated in vacuo below 40° C. and dried under high vacuum below 40° C. for 1 hr to provide NRH (17.1 g and 97% purity by LC-MS), which was stored at −20° C. under N₂.

### Example 2. NRH Reduced Production of Inflammatory Cytokines in an Ex Vivo Polyclonal Immune Activation Model

Heparinized venous blood was collected from healthy human adult donors. Peripheral blood mononuclear cells (PBMCs) were isolated by Ficoll-Hypaque density-gradient centrifugation. PBMCs (1.0×10⁶) were seeded in round-bottom, 96-well plates in 200 μL of medium, unstimulated or stimulated with an anti-CD3 antibody (1 μg/mL, clone-HIT3a, Biolegend) plus an anti-CD28 antibody (1 μg/mL, clone-CD28.2, Biolegend) to induce T-cell activation, and incubated in cell-culture medium. The PBMCs were unstimulated or stimulated with anti-CD3 and anti-CD28 antibodies to induce T-cell activation, in the absence or presence of 250 μM NRH. After 18 hours of incubation, lymphocytes were analyzed for intracellular levels of IFN-γ, TNF-α and IL-2 by flow cytometry. Intracellular cytokine production was stopped by treating the cells with Golgi stop after 1 hr of treatment.

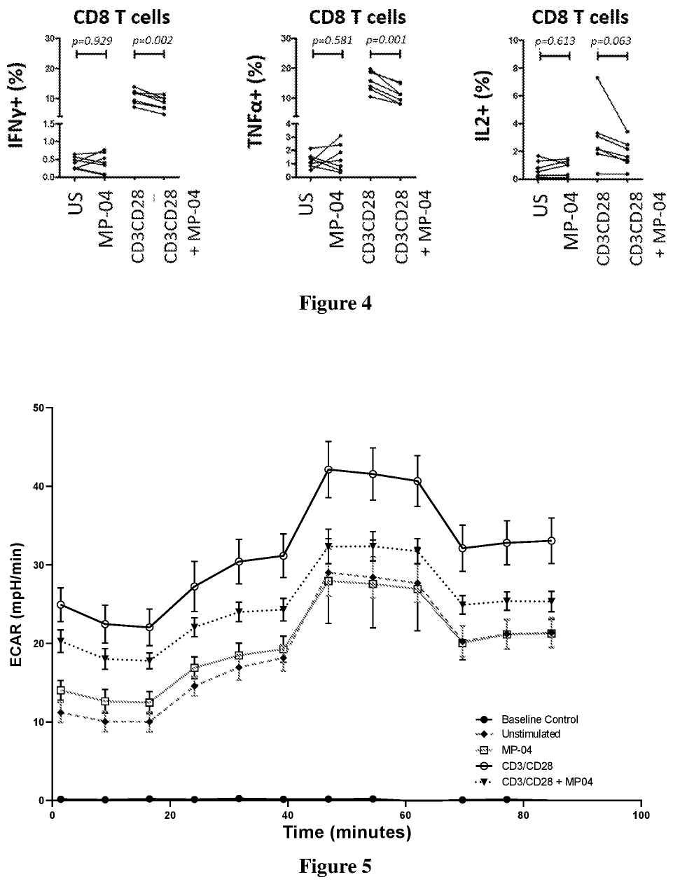

FIG. 4 shows that CD8⁺ T cells stimulated with anti-CD3 and anti-CD28 antibodies produced significantly or markedly more IFN-γ, TNF-α and IL-2 than unstimulated (US) CD8⁺ T cells, and NRH (MP-04) significantly reduced the production of IFN-γ, TNF-α and IL-2 in activated CD8⁺ T cells (p<0.05 in the Mann-Whitney U test).

### Example 3. NRH Reduced Basal Extracellular Acidification Rate (ECAR, a Measure of Glycolysis) in Activated PBMCs

PBMCs (1.5×10⁶) obtained from a healthy human adult donor were unstimulated or stimulated with anti-CD3 and anti-CD28 antibodies (1 μg/mL each) to induce T-cell activation, in the absence or presence of 250 μM NRH for 4 hr. The cells were then washed and plated on poly-D-lysine-coated Seahorse XF microplates at a density of 3×10⁵ cells per well (5 replicates). The plates were centrifuged briefly and incubated at 37° C. in a non-CO₂ incubator for 30 min to settle the cells in the bottom of the plate. Extracellular acidification rate (ECAR, a measure of glycolysis) and oxygen consumption rate (OCR, a measure of oxidative phosphorylation) were determined using a Seahorse XF cell mito stress test kit and a Seahorse Xfe96 analyzer (Agilent Technologies, Santa Clara, Calif.) under basal condition or in response to 1.0 μM oligomycin (an inhibitor of ATP synthase), 1.5 μM carbonyl cyanide-p-trifluoromethoxyphenylhydrazone (FCCP, a mitochondrial uncoupler), 0.5 μM rotenone (an inhibitor of electron transport at complex I) and antimycin A (an inhibitor of electron transport at cytochrome c reductase). PBMCs include monocytes and lymphocytes including T cells, B cells and natural killer cells.

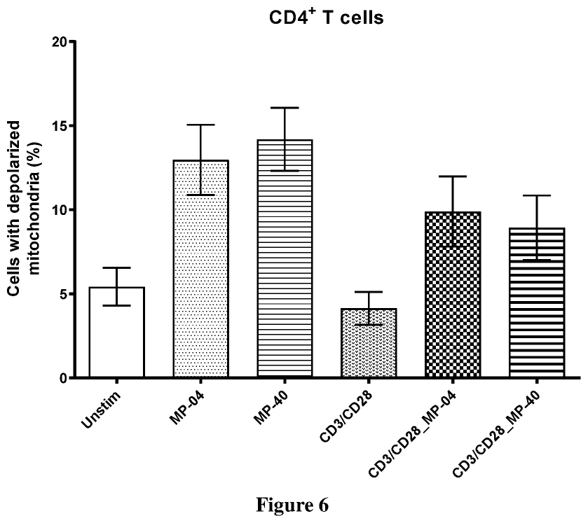

FIG. 5 shows that PBMCs from the human donor stimulated with anti-CD3 and anti-CD28 antibodies had a markedly higher extracellular acidification rate (ECAR, a measure of glycolysis) than unstimulated PBMCs, and NRH (MP-04) significantly reduced ECAR in activated PBMCs.

### Example 4. NRH and NRHTA Induced Mitochondrial Depolarization in T Cells and Reduced Cell Death in T Cells

PBMCs (10⁶ cells per test group) obtained from healthy human adult donors were unstimulated or stimulated with anti-CD3 and anti-CD28 antibodies to induce T-cell activation, in the absence or presence of 250 μM of NRH or NRH-triacetate (NRHTA). Measurements were made at 5, 15 and 24 hr after treatment.

After the indicated time of incubation, the PBMCs were stained with anti-CD3, anti-CD4 and anti-CD8 antibodies (Biolegend), JC-1 (1 μM, Thermo Fisher) and annexin V (Thermo Fisher) prior to analysis by flow cytometry. Annexin V binds to cell-surface phosphatidylserine, which is a marker for different forms of cell death including apoptosis and necrosis. The stained cells were re-suspended in phosphate-buffered saline (PBS) and acquired in a Cytek Aurora flow cytometer using a FlowJo software (v10) for analysis. About 200,000-300,000 cells were acquired in the flow cytometer for analysis. Standard procedures for spectral compensation were performed to obtain cell populations. Lymphocytes identified by forward and side scatter were gated to obtain CD3⁺/CD4⁺ and CD3⁺/CD8⁺ T-cell populations. Those T-cell populations were gated to identify the red and green signals for JC-1 aggregate and monomer, respectively, and annexin V. The percentage of cells staining green by JC-1 monomer was used to determine the percentage of cells with depolarized mitochondria. The percentage of cells staining with annexin V was used to determine the percentage of cells subject to different forms of cell death including apoptosis and necrosis.

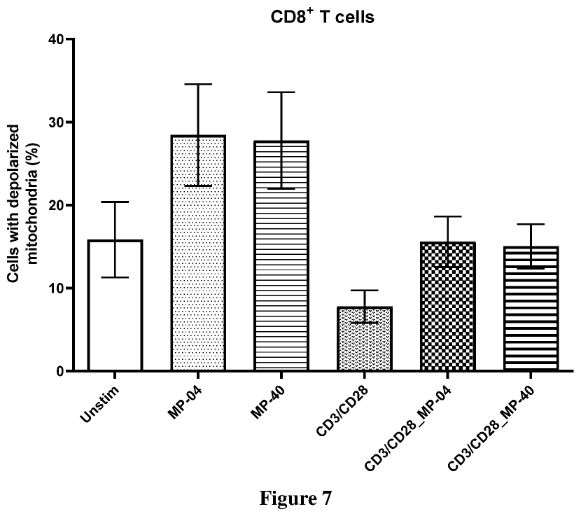

FIGS. 6 and 7 show that incubation with NRH (MP-04) and NRHTA (MP-40) for 24 hr significantly induced mitochondrial membrane depolarization in CD4⁺ and CD8⁺ T cells, respectively, unstimulated or stimulated with anti-CD3 and anti-CD28 antibodies.

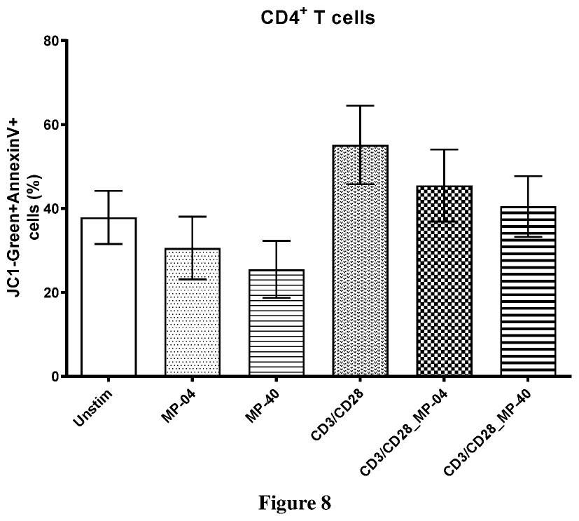

FIGS. 8 and 9 show that incubation with NRH (MP-04) and NRHTA (MP-40) for 24 hr reduced cell death including apoptosis of CD4⁺ and CD8⁺ T cells, respectively, with depolarized mitochondria and unstimulated or stimulated with anti-CD3 and anti-CD28 antibodies.

### Example 5. NRH and NRH-Triacetate Reduced H₂O₂-Induced Hemolysis In Vitro

Individual test compounds were dissolved in 0.9% normal saline to prepare 0.3 M stock solutions. A 0.3 M stock solution was added to a 20% suspension of washed red blood cells (RBCs) in saline to obtain final concentrations of 2000 μM, 200 μM and 20 μM of the compound. The RBC suspensions were incubated with the test compound at 37° C. for 1 hr prior to testing. A 2.5% H₂O₂ solution was prepared fresh by dilution with normal saline and chilled at 2-8° C. before use. Each RBC suspension containing the test compound was split into tubes A and B. 250 μL of the chilled H₂O₂ solution was added to 250 μL of RBC suspension in each tube while maintaining the tubes in an ice bath. The tubes were incubated at 37° C. for 4 hr, and then 4.5 mL of saline or de-ionized water was added to tube A or tube B, respectively. After the tubes were incubated for 10 min, the mixture in the tubes was centrifuged at 1800 g, and then absorbance was measured in the supernatant at 540 nm. The percentage of hemolysis was calculated as:

% Hemolysis=Absorbance_{540 nm} in saline (tube A)/Absorbance_{540 nm} in de-ionized water (tube B)

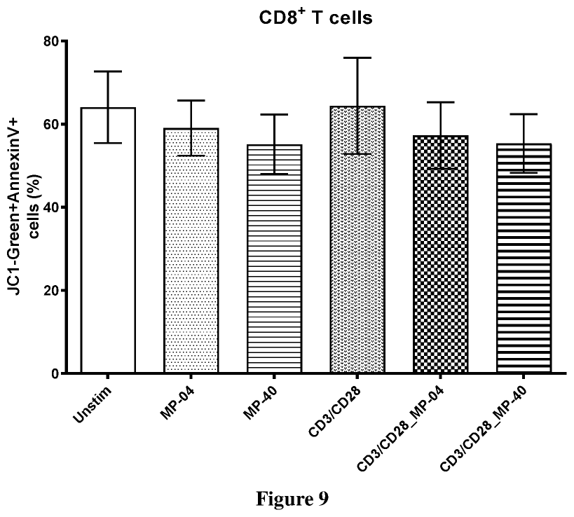

FIG. 10 shows that both NRH (MP-04) and NRH-triacetate (MP-40), but neither NR (MP-02) nor NR-triacetate (MP-39) at any concentration tested, reduced H₂O₂-induced hemolysis in the in vitro assay.

### Example 6. NRH Protected Hemoglobin from H₂O₂-Induced Oxidative Changes In Vitro

A 4 M stock solution of sodium azide was prepared. One mL of the 4 M stock azide solution was diluted with 9 mL of phosphate-buffered saline (PBS, pH 7.4) to obtain a 0.4 M stock solution, which was further diluted with PBS to obtain a 0.01 M working solution of sodium azide. Eight mL of whole blood was collected in buffered acid citrate dextrose tubes from a healthy human donor. PBS was added to 500 μL of whole blood. The samples were centrifuged, and then the supernatant from each tube was discarded to obtain a red blood cell (RBC) pellet.

Ten μL of 0.01 M sodium azide working solution was added to 20 μL of RBC suspension. Twenty μL of hydrogen peroxide (H₂O₂) diluted in PBS was added to the RBC suspension to obtain final concentrations of H₂O₂ ranging from 0.01 mM to 100 mM. The samples were incubated at 37° C. for 1.5 hr. 950 μL of de-ionized water was added to the samples, which were then incubated for 10 min to lyse the RBCs and obtain hemoglobin upon centrifugation at 1,500 g. Spectral scans of the lysed samples between 200 nm and 1100 nm were obtained in 2 nm increments, using a Systronics® dual-beam UV-spectrophotometer and de-ionized water as a blank. Samples incubated with PBS at 37° C. for 1-1.5 hr were used as controls to establish the oxidative effects of H₂O₂ on RBC hemoglobin. Absorbance at 576 nm is a measure of hemoglobin concentration in a sample, and oxidation of hemoglobin to methemoglobin results in a drop in absorbance at 576 nm and an increase in absorbance at 630 nm. Fantao et al., Anal. Biochem., 521:11-19, (2017).

Stock solutions of NRH (29.6 mM) and NR (18.3 mM) were prepared in 0.9% normal saline. Appropriate quantities of the stock NRH or NR solution and PBS were added to 500 μL of whole blood to obtain final concentrations of NRH or NR ranging from 1 μM to 1000 μM. The whole blood samples with NRH or NR were mixed gently and incubated at 37° C. for 1-1.5 hr. The samples were centrifuged, and then the supernatant was discarded to obtain an RBC pellet for each sample.

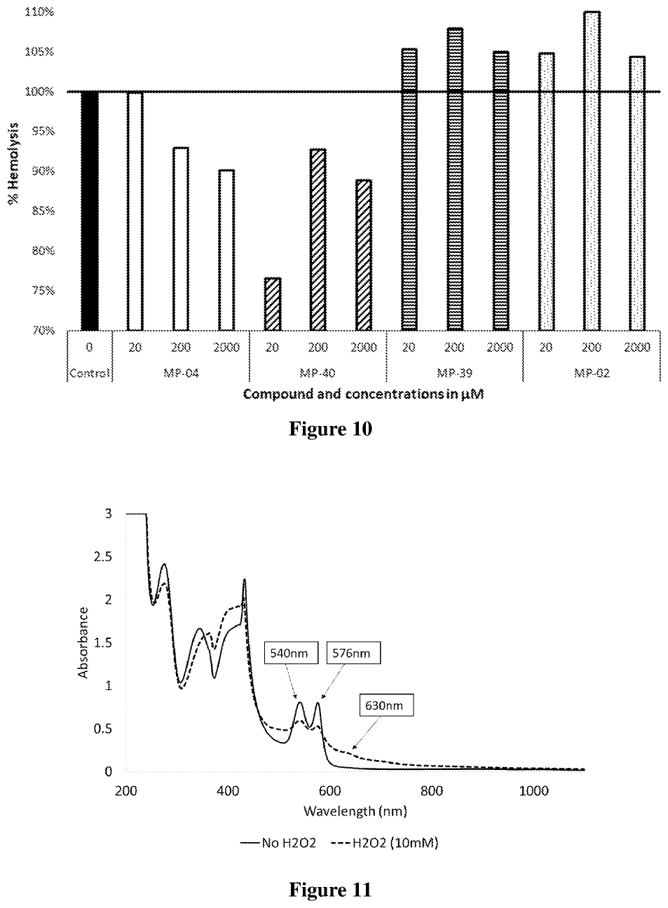

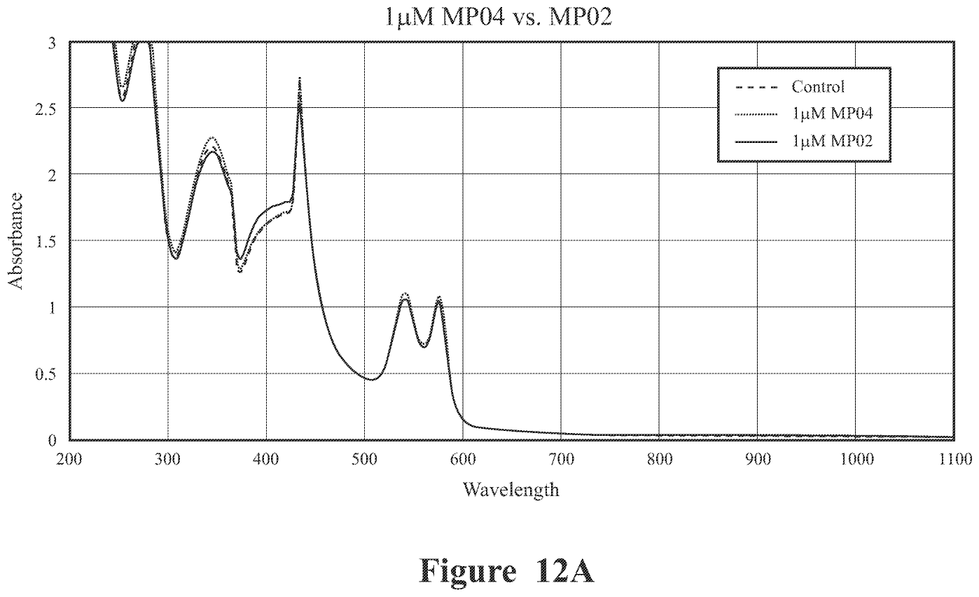

FIG. 11 shows that 10 mM H₂O₂ caused oxidative changes to hemoglobin which reduced the amplitude of absorbance peaks at 576 nm, 540 nm, 434 nm, 348 nm and 270 nm. FIG. 12A-C shows that pre-incubation of RBCs with 1, 10 and 100 μM, respectively, of NRH (MP04), but not with NR (MP02), protected hemoglobin from 1 mM H₂O₂-induced oxidative changes, as pre-incubation with NRH increased the amplitude of absorbance peaks at 576 nm, 540 nm, 434 nm, 348 nm and 270 nm.

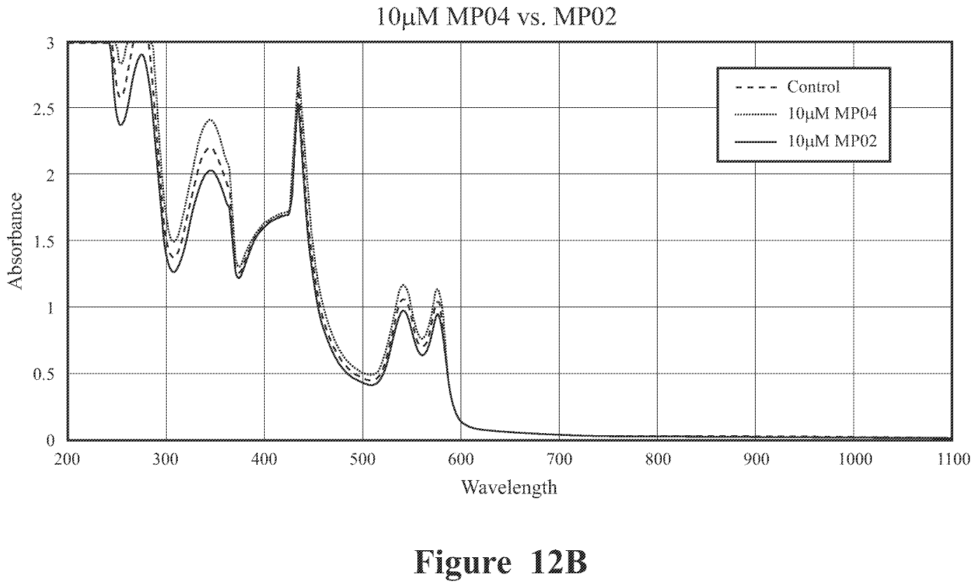

Because absorbance at 576 nm and 540 nm is characteristic of hemoglobin and absorbance at 630 nm is characteristic of methemoglobin, the ratio of absorbance at 576 nm to absorbance at 630 nm (A₅₇₆/A₆₃₀) is a measure of the hemoglobin/methemoglobin ratio. FIG. 13 shows that exposure of RBCs to 1 mM H₂O₂ significantly reduced the A₅₇₆/A₆₃₀ ratio, and treatment of RBCs exposed to 1 mM H₂O₂ with 1 μM or 100 μM NRH (MP-04) restored the A₅₇₆/A₆₃₀ ratio. Treatment of RBCs exposed to H₂O₂ with NRH significantly increased, or restored, the A₅₇₆/A₆₃₀ ratio, but treatment with NR did not significantly affect the A₅₇₆/A₆₃₀ ratio (data not shown).

### Example 7. NRH Increased the NADH/NAD⁺ Ratio in H₂O₂-Exposed HEK293 Cells In Vitro

HEK293 cells were trypsinized and plated at a density of 60,000 cells per well. The cells were then treated with H₂O₂ (600 μM prepared with Dulbecco's Modified Eagle Medium [DMEM]) for 30 min. The media was then replaced with serum media (DMEM supplemented with 10% fetal bovine serum [FBS]) containing NRH or NR, and were incubated at 37° C. under 6% CO₂ for 30 min or 6 hr. The cells were incubated with the following concentrations of NRH or NR: 0.01, 0.1, 1.0, 10, 100 and 1000 μM. NAD+ and NADH levels at 30 min and 6 hr were estimated using the NAD/NADH-Glo Promega Bioluminescent assay. The results were expressed as fold change relative to the values obtained with untreated cells.

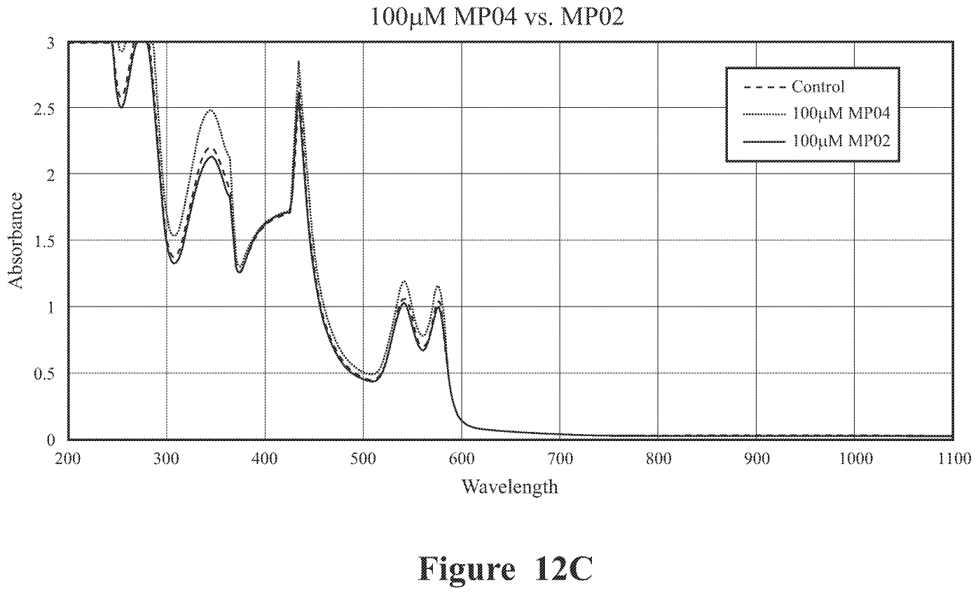

FIGS. 14A and B shows that 30 min and 6 hr, respectively, of incubation with NRH (MP-04) at 100 and 1000 μM significantly increased the NADH/NAD⁺ ratio in HEK293 cells exposed to H₂O₂, while NR (MP-02) at all tested concentrations did not significantly affect the ratio.

### Example 8. NRH and NRH-Triacetate are Much More Stable in Human Serum than NR

Three blood samples were collected from healthy human donors with the same blood group in serum separator vacutainer gel tubes. The samples were centrifuged at 1,800 g to separate serum. The serum samples obtained were pooled together into a separate tube.

NR, NRH and NRH-triacetate (NRHTA) were initially dissolved in DMSO and then diluted with normal saline to obtain a 1 mM stock solution of each compound. 100 μL of stock solution was added to 900 μL of pooled serum to assess the stability of the test compound in human serum. 200 μL of saline with 1,800 μL of pooled serum was used to prepare a solution for blanking and as a reference measurement. The stock solutions were diluted in the pooled serum and normal saline to prepare 100 μM solutions of the test compounds.

In previous experiments, spectral scans between 190 nm and 1100 nm were performed to determine absorbance maxima for NR, NRH and NRHTA, which have maximal absorbance at 265 nm, 340 nm and 331 nm, respectively, in solution.

The stability of a test compound in human serum was determined by a change in the concentration of the compound as measured by a change in absorbance at the maximal absorbance wavelength for the compound as a function of time. The test compounds at 100 μM were incubated in human serum at 37° C. Absorbance measurements were made at 0 min (T0), 5 min, 30 min, 120 min, 240 min, 360 min and 1440 min (24 hr). The relative change in absorbance compared to T0 was plotted and used for statistical analysis. The analysis was performed in triplicates.

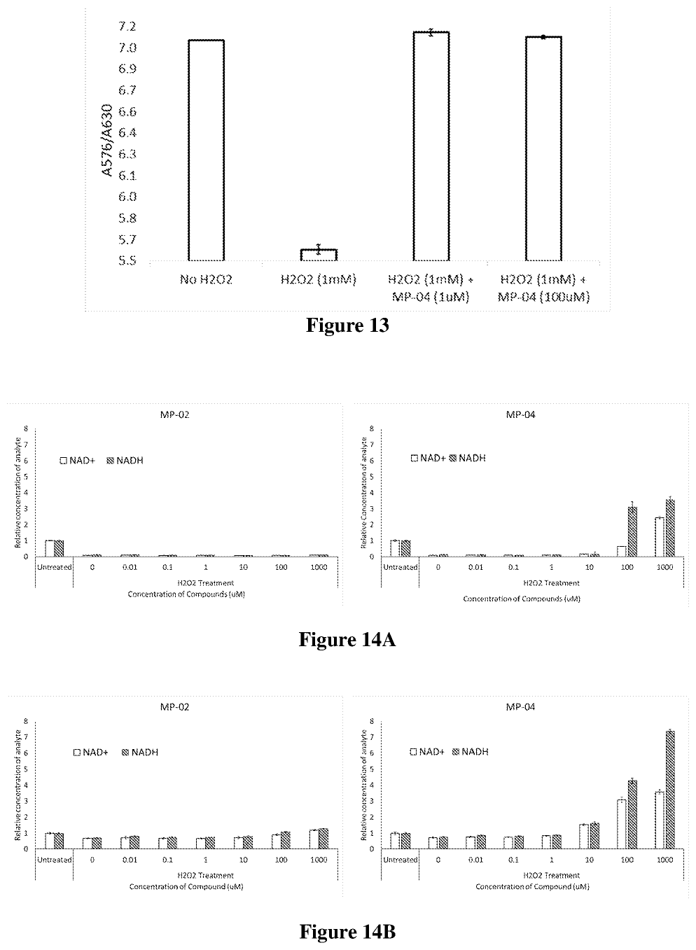

FIG. 15 shows that both NRH (MP04) and NRH-triacetate (MP40) were much more stable in human serum than NR (MP02) in the in vitro assay (*=p<0.05 for NR versus NRH and NRH-triacetate; #=p<0.05 for NRH-triacetate versus NRH). Most of both NRH and NRH-triacetate remained intact after 6 hr in human serum, whereas most of NR did not remain intact within 5 min of addition to human serum as measured by a sharp drop in absorbance at 265 nm. Surprisingly, about 50% of NRH-triacetate remained intact after 24 hr in human serum.

### Example 9. Intraperitoneally Injected NRH Distributed to the Kidney and Liver in a Rat

Freshly weighed NRH (100 mg) was dissolved in 500 μL sterile IP water. The NRH solution was intraperitoneally injected into a healthy, male Wistar Han rat (200 g) at a single dose of 500 mg/kg. 500 μL of sterile IP water was intraperitoneally injected into another healthy, male Wistar Han rat (200 g) as a control.

The animals were euthanized by cervical dislocation under anesthesia after 4 hr. Whole blood was collected in K₂EDTA vacutainer tubes by cardiac puncture using a 2 mL syringe. The liver and kidney were harvested subsequently by dissection. The tissues were placed in a petridish, rinsed with ice-cold saline, and then weighed. 72 mg of liver of the treated animal and 74.3 mg of liver of the untreated animal were homogenized, and 71.6 mg of kidney of the treated animal and 70.7 mg of kidney of the untreated animal were homogenized. Homogenization of the tissues was carried out separately in 3 mL of ice-cold methanol (about 20 mg/mL of tissue in methanol). The homogenate was snap-frozen in liquid nitrogen and maintained at −80° C. for analysis. The blood samples were maintained at 2-8° C. for analysis.

250 μL of whole blood from the K₂EDTA tubes was added to a tube containing 50 μL of 500 ng/mL of tolbutamide as the internal standard. 600 μL of ice-cold HPLC-grade methanol was added to this mixture for extraction of nicotinamide adenine dinucleotide (NAD⁺) and NRH. The resulting mixture was mixed well and centrifuged at 12500 rpm for 10 min. The supernatant was measured for NAD⁺ and NRH by LCMS/MS. For the liver and kidney tissues, 250 μL of the tissue homogenate was processed in the same manner.

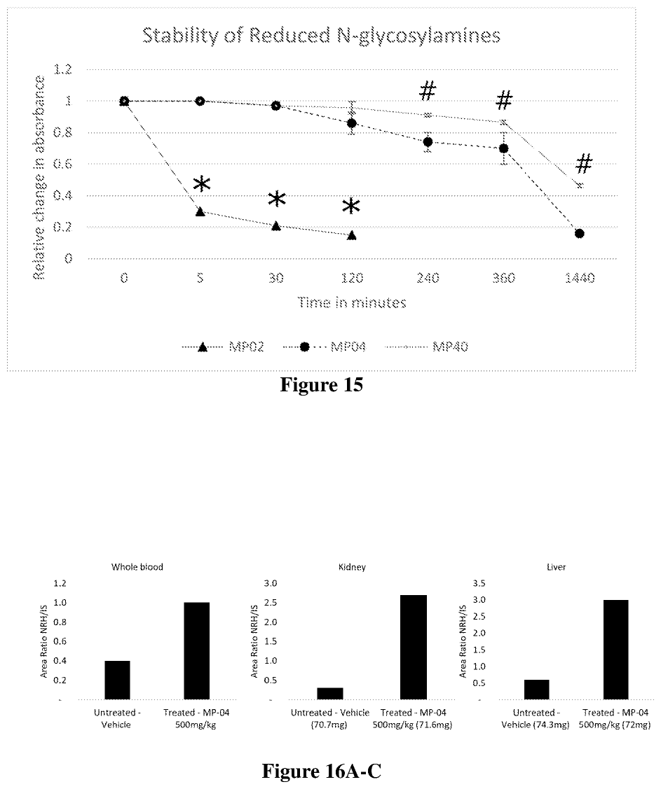

FIG. 16A-C shows that a single intraperitoneal injection of NRH (MP-04) into a Wistar Han rat at a dose of 500 mg/kg resulted in increased concentrations of NRH in whole blood, the kidney and the liver, respectively, after 4 hr as compared to the corresponding concentrations in a Wistar Han rat intraperitoneally injected with vehicle. In FIG. 16A-C, the “area ratio NRH/IS” is the ratio of the peak area of NRH to the peak area of internal standard (tolbutamide), and is directly proportional to the concentration of NRH.

### Example 10. Assessment of NRH in a Mouse Model of Sepsis

16 wild type C57BL/6 are randomized into two groups of eight animals each. Animals in Group 1 receive 5 mg/kg of lipopolysaccharide (LPS) intraperitonially to induce sepsis, while animals in Group 2 do not. In each group, four animals each are treated with NRH at 25 mg/kg administered intraperitoneally one hour prior to administration of LPS, and four animals receive a control. Blood sampling is done at dosing and 6 hours post dose. Animals are euthanized at 6 hours to harvest tissues of lung, liver, kidney, muscle and whole blood.

NAD⁺, NRH, aspartate transaminase (AST), alanine transaminase (ALT), creatinine, lactate dehydrogenase (LDH), lactate, blood count (CBC), WBC phenotyping, cytokines (TNF-α, IFN-γ and IL-2) are measured in blood samples. Histopathology evaluation and NAD⁺ quantification are done in the tissue samples. NAD⁺ is measured using established LCMS/MS methods. LDH, creatinine, AST, ALT, LDH are measured using commercially available auto-analyzer based assays. Cytokines are measured using enzyme-linked immunosorbent assay (ELISA). WBC phenotyping is done using flow cytometry.

14 wild type C57BL/6 mice are used for the second study. The animals are divided into two groups of 7 animals each, receiving LPS (5 mg/kg intraperitoneally) or LPS (5 mg/kg intraperitoneally) and 250 mg/kg of NRH. NRH in the second group is given intraperitoneally 30 minutes before the LPS dose. Time to mortality and time to recovery is observed for a period of 1 week to establish the efficacy of NRH. Samples of tissues such as liver, kidney, lung and muscle would be collected after 7 days or at death to evaluate the changes in gross morphology and histopathology.

### Example 11. Assessment of NRH in a Mouse Model of Acute Kidney Injury

8-week-old C57BL/6 mice are injected with either vehicle (saline) or cisplatin (20 mg/kg) simultaneously with either the vehicle comprising of phosphate buffered saline (PBS) or test compound which is IV NRH at doses of 50 mg/kg and or 250 mg/kg. 5 animals per group are used for the study. At 72 hours after the initiation of the experiment, the mice receive either vehicle or NRH. Repeat doses are administered every 24 hours. The animals are sacrificed 4 h after the last injection. The blood samples are collected for BUN and creatinine measurements, and renal tissue collected for histology, tissue measurements (NAD⁺, NADH and NRH) and assessment of casts. The efficacy is assessed based on changes of serum creatinine and urinary casts. Whole blood NAD⁺, NADH and NRH levels are assessed at baseline and at the time of animal sacrifice. Creatinine is evaluated using modified Jaffe's technique. BUN is measured using enzymatic methods (urease). NAD⁺, NADH and NRH measurements are carried out using established LCMS/MS method.

### Example 12. Assessment of NRH in a Rat Model of Methemoglobinemia

Methemoglobinemia is induced in Sprague Dawley rats by an intravenous amyl nitrate. There are 3 animals per group (and 15 in total), with one group serving as control. NRH is administered 30 minutes before administration of 1 mg/mL amyl nitrate as per standard models (Klimmek et al., Arch Toxicol., 1988). The protective effects of 0, 5, 50, 125 and 250 mg/kg of MP-04 is evaluated by measurement of methemoglobin values in whole blood at pre-dose (5 minutes before administration of amyl nitrate), 10 minutes, 30 minutes, 1 hour and 2 hours using standard sphectrophotometric assays.

### Example 13. Pharmacokinetics of NRH in Sprague-Dawley Rats

The primary purpose of this study is to investigate the plasma and tissue (liver and kidney) pharmacokinetics (PK) of NRH, following slow-bolus intravenous (IV) administration in male Sprague-Dawley Rats. This study includes 4 groups of 3 male rats (12 total) weighing around 300-325 grams at the time of surgical catheter placement. There is a vehicle control group (Group 1), and 3 dose groups low to high (Groups 2, 3, and 4) dosed at 50, 125, or 250 mg/kg, respectively.

On Day 1, all rats are administered NRH IV slow bolus dose (1st dose) over a period of 1-2 minutes via one jugular venous catheter. Catheter are marked prior to dosing to ensure that the catheter used for dosing is separate than the catheter used for blood collections. The catheter is flushed with 0.3 mL sterile saline in order to ensure the entire dose is administered. Animals are not be fasted prior to dosing. Blood is collected (200 μL, K₂EDTA, via the 2nd jugular vein catheter) pre-dose and at 0.5, 1, 2, 4, 8, 12, and 24-hours post-dose. The blood samples are snap frozen and maintained at −80° C. until analysis. Body weights are recorded prior to dose on Day 1 and 24 hours later. After the animals are weighed at around 24 hours, on Day 2, all rats are administered the second dose of NRH IV slow bolus dose over a period of 1-2 minutes via the jugular venous catheter marked for dosing. The catheter is flushed with 0.3 mL sterile saline in order to ensure the entire dose is administered. Clinical observations are recorded whenever an abnormality is seen in this short-term study. Animals are observed continually during the first 4 hours post dose. Additional cage-side general health/morbidity/mortality observations are conducted by animal care at least once daily. All rats are euthanized at 4-hours post second dose. Liver and kidneys from each animal are collected, weighed immediately, and snap-frozen in liquid nitrogen and stored at −80° C. until analysis.

Whole blood and tissue levels of NRH, NAD+ and nicotinamide adenine dinucleotide hydrogen (NADH) is evaluated by LCMS/MS. Pharmacokinetic measurements of NRH at the doses administered related to C₀, AUC₀₋ₜ, AUC_{0-inf}, T_{1/2}, Kₑₗ, MRTₗₐₛₜ, Vss, Cl are evaluated using Phoenix WinNonlin Software.

### Example 14. Pharmacokinetics and Pharmacodynamics Following Multiple Ascending Dose of NRH in Beagle Dog

The objective of this study is to assess the systemic toxicity of NRH following intravenous injection dose escalation to Beagle dogs to determine the Maximum Tolerated Dose (MTD) (Phase I) and followed by repeat dose study to assess the toxicological profile including toxicokinetic profile of NRH (Phase II) when administered to dogs.

There are four groups of Beagle dogs, with one group serving as control. Each group is having 1 male and 1 female Beagle dogs. The animals are acclimatized for 5-7 days. Four escalating doses are administered to the animals with a two-day washout between doses until the highest dose or until the maximum tolerated dose is reached. The starting dose is 25 mg/kg is used. After the maximum tolerated dose is established, a 10-Day repeat dose phase is conducted, and doses are decided based on the outcome of escalation dose study. Two dose levels are spaced and tested for toxicity and kinetic profile.

Observations including clinical parameters, body weight, body temperature, food consumption, ECG, urinalysis, clinical chemistry, hematology and coagulation parameters are performed 24 hours after each dosing. Blood samples are collected pre-dose. Blood samples are collected from each dose administration in Phase I and in Phase II will be on Days 1 and 10 at Predose (0 h) and 0.083, 0.25, 0.5, 0.75, 1, 2, 4, 8, and 24 hours (h) post-dose. The liver and kidney samples are obtained at the end of the study. All the tissue and blood samples are used for pharmacokinetic measurements (NRH concentrations along C₀, AUC₀₋ₜ, AUC_{0-inf}, T_{1/2}, Kₑₗ, MRTₗₐₛₜ, Vss, Cl are evaluated using Phoenix WinNonlin Software) and pharmacodynamic measurements (NAD+ and NADH measurements using LCMS/MS).

### Example 15. Single Ascending Dose (SAD) and Multiple Ascending Dose (MAD) Studies of NRH in Humans

Safety, tolerability and pharmacodynamics (PD) of NRH are evaluated using a standard SAD and MAD design. Briefly, in a SAD study, 40 healthy volunteers are sequentially randomized 8:2 to intravenously receive a single ascending dose of NRH (1, 3, 10 or 30 mg/kg) or placebo (normal saline) over 30 min. Study criteria for healthy volunteers include 18-65 years old, body mass index (BMI)<35, no significant co-morbidity, and normal hematology and chemistry values.

After the safety of NRH is established in the SAD study, a standard MAD study is conducted to determine the maximum tolerated dose in a healthy cohort. Briefly, in a MAD study, 40 healthy volunteers are sequentially randomized 8:2 to intravenously receive daily doses of NRH (1, 3, 10 or 30 mg/kg) or placebo (normal saline) over 30 min for 7 days. Subjects from the SAD study are eligible to participate in the MAD study after a 7-day washout period.

The pharmacokinetics (PK) of NRH and metabolites thereof (e.g., NR, nicotinamide [Nam] and N-methylnicotinamide [MeNam]) in plasma and whole blood is analyzed. PD analysis includes measurement of NAD⁺ and NADH levels and the NAD⁺/NADH ratio in whole blood and peripheral blood mononuclear cells (PBMCs), including T cells. In addition, pro-inflammatory cytokines and other markers of T-cell activation are measured to determine the biological activity of NRH in patients with liver impairment (Child-Turcotte-Pugh Score A [CTP-A] and Child-Turcotte-Pugh Score B [CTP-B] patients). Measurements are performed at baseline and post-dosing at 1 hr, 2 hr, 4 hr, 8 hr and 24 hr in the single-dose and multiple-dose cohorts.

### Example 16. Phase Ib Study of NRH in Patients with Cirrhosis

After the safety and tolerability of intravenously administered NRH are established in a MAD study among healthy volunteers, a Phase Ib study is conducted among patients with cirrhosis (Child-Pugh Score A [CP-A] and Child-Pugh Score B [CP-B] patients, N=8 each) and alcoholic liver disease (ALD). The patients are intravenously injected with 10 mg/kg of NRH daily for 7 days.

The pharmacokinetics of NRH and its metabolites, including their levels in plasma and whole blood, is analyzed using MS. Levels of NRH and its metabolites in PBMCs including T cells, and pro-inflammatory cytokines and other markers of T-cell activation, are measured to determine the biological activity of NRH in patients with liver impairment (CP-A and CP-B patients). Measurements are performed at baseline and post-dosing at 1 hr, 2 hr, 4 hr, 8 hr and 24 hr in the single-dose and multiple-dose cohorts. Pharmacology assessments are examined with descriptive statistics. Statistical analyses are performed using SAS version 9.4 (SAS Institute, Cary, N.C.). Missing values are not replaced or estimated. Descriptive statistics are used to characterize the demographics and other clinical variables. Categorical variables are compared using a chi-squared test or Fisher's exact test (when expected cell counts are <5). Medians are reported with interquartile ranges and compared using a Wilcoxon rank sum test. Plasma concentrations of NRH and its metabolites and whole blood concentrations of NAD⁺ and its metabolites across treatment cohorts are compared by analysis of variance of log-transformed or rank values.

### Example 17. Phase Ib Study of NRH in Patients with SIRS in Covid-19

After the safety and tolerability of intravenously administered NRH are established in a MAD study among healthy volunteers, a Phase Ib study is conducted among clinically stable patients with an established diagnosis of covid-19 RT-PCR in the last 24 hours, with a computed tomography (CT) score categorizing the disease severity as moderate. 200 patients are recruited for this study.

Patients are randomized into two groups—one group receiving standard-of-care (SOC) and the second group receiving standard-of-care with IV NRH (10 mg/kg) for up to 7 days. Measurements of side effects and safety profile and PK-PD are estimated after 14 days from the date of diagnosis as a primary objective. Time to resolution of symptoms, symptoms severity score, requirement of oxygenation or ventilation, hospitalization and death would be estimated as a secondary outcome measure in this study.

While various embodiments of the present disclosure have been described, such embodiments are provided by way of illustration and example only. Numerous variations thereof and modifications thereto will be apparent to those skilled in the art and are encompassed by the present disclosure. It is understood that various alternatives to the embodiments of the disclosure can be employed in practicing the disclosure and are encompassed by the disclosure.

What is claimed is:

1. A method of treating an immune-related disorder, a kidney disorder, a liver disorder, a hemolytic disorder, or a disorder or condition associated with oxidative stress, damage or injury, comprising administering to a subject in need thereof a therapeutically effective amount of dihydronicotinamide riboside (NRH), dihydronicotinic acid riboside (NARH) or a reduced derivative thereof, or a pharmaceutically acceptable salt, solvate, hydrate, clathrate, polymorph or stereoisomer thereof.

2. The method of claim 1, wherein the concentration of NRH, NARH or a reduced derivative thereof is about 1-1000 μM, about 1-500 μM, or about 500-1000 μM, or about 1-250 μM, about 250-500 μM, or about 500-750 μM or about 750-1000 μM.

3. The method of claim 1, wherein the concentration of NRH, NARH or a reduced derivative thereof is about 1-200 μM, about 1-150 μM, about 1-100 μM, about 100-200 μM, about 1-50 μM, about 50-100 μM, about 100-150 μM or about 150-200 μM.

4. The method of claim 1, wherein the concentration of NRH, NARH or a reduced derivative thereof persists for at least about 1 hr, about 2 hr, about 3 hr, about 6 hr, about 8 hr or about 12 hr after administration.

5. The method of claim 1, wherein NRH, NARH or a reduced derivative thereof is administered intravenously or subcutaneously as a bolus one, two, three or four times daily, or by continuous infusion.

6. The method of claim 5, wherein the amount of NRH, NARH or a reduced derivative thereof administered is about 0.1-60 mg/kg, about 0.5-50 mg/kg or about 1-40 mg/kg, about 1.5-30 mg/kg per day, or about 1-4000 mg, about 50-3500 mg, about 100-3000 mg or about 100-2000 mg per day.

7. The method of claim 5, wherein the therapeutically effective amount of NRH, NARH or a reduced derivative thereof administered is about 1-1000 mg, about 1-500 mg or about 500-1000 mg per day, or about 1-50 mg, about 50-100 mg, about 100-200 mg, about 200-300 mg, about 300-400 mg, about 400-500 mg, about 500-750 mg or about 750-1000 mg per day.

8. The method of claim 1, wherein NRH, NARH or a reduced derivative thereof has the beta-D-riboside configuration.

9. The method of claim 1, wherein NRH, NARH or a reduced derivative thereof is stereoisomerically pure.

10. The method of claim 1, wherein NRH, NARH or a reduced derivative thereof has the beta-D-riboside configuration and an approximately 1:1 ratio of beta-/alpha-anomers.

11. The method of claim 1, further comprising administering one or more additional therapeutic agents.

12. The method of claim 11, wherein the one or more additional therapeutic agents comprises an antioxidant or/and an anti-inflammatory agent.

13. The method of claim 12, wherein the antioxidant comprises a vitamin or an analog thereof glutathione (GSH) or a derivative thereof or an antioxidant which increases glutathione level or a mitochondria-targeted antioxidant or combinations thereof.

14. The method of claim 12, wherein the anti-inflammatory agent comprises an NSAID, a glucocorticoid, an immunosuppressant, or an inhibitor of pro-inflammatory cytokine(s) or receptor(s) therefor or combinations thereof.

15. A pharmaceutical composition comprising one or more pharmaceutically acceptable excipients or carriers, and dihydronicotinamide riboside (NRH), dihydronicotinic acid riboside (NARH) or a reduced derivative thereof, or a pharmaceutically acceptable salt, solvate, hydrate, clathrate, polymorph or stereoisomer thereof, wherein the composition is in a lyophilized (freeze-dried) form.

16. The pharmaceutical composition of claim 15, wherein the one or more pharmaceutically acceptable excipients or carriers comprise an amino acid or/and a stabilizing agent (and optionally a bulking agent.

17. The pharmaceutical composition of claim 15, wherein NRH, NARH or a reduced derivative thereof is mixed, dissolved or suspended in an aqueous buffer having a pH of about 7.4-10.5, about 8-10.5 or about 9-10.5 prior to lyophilization.

18. The pharmaceutical composition of claim 17, wherein the aqueous mixture, solution or suspension comprising NRH, NARH or a reduced derivative thereof is sterilized by filtration through a membrane having a pore size of no more than about 0.2 micron prior to lyophilization.

19. The pharmaceutical composition of claim 15, which is stored in a hermetically sealed, colored vial or ampule made of glass or plastic.

20. The pharmaceutical composition of claim 19, wherein the vial or ampule is under vacuum or under an inert gas.
